(19) United States
(12) Patent Application Publication
(10) Pub. No.: US 2022/0362220 A1
(43) Pub. Date: Nov. 17, 2022

(54) 5-DEUTERO-THIAZOLIDINE-2,4-DIONE COMPOUNDS AND METHODS OF TREATING MEDICAL DISORDERS USING SAME

(71) Applicant: Poxel SA, Lyon (FR)

(72) Inventors: Sheila DeWitt, Auburn, NH (US); Vincent Jacques, Somerville, MA (US); Leonardus van der Ploeg, Newton, MA (US)

(21) Appl. No.: 17/094,942

(22) Filed: Nov. 11, 2020

Related U.S. Application Data
(63) Continuation of application No. 16/541,337, filed on Aug. 15, 2019, now abandoned.
Application No. 16/541,337 is a continuation of application No. 15/705,544, filed on Sep. 15, 2017, now abandoned.
Application No. 15/705,544 is a continuation of application No. PCT/US2016/023008, filed on Mar. 18, 2016.
(60) Provisional application No. 62/135,995, filed on Mar. 20, 2015.

(51) Int. Cl. A61K 31/427; C07D 417/12; C07D 277/34; C07B 59/00; A61P 3/10; C07D 417/10

(52) U.S. Cl. CPC A61K 31/427; C07D 417/12; C07D 417/10; C07B 59/002; A61P 3/10; C07D 277/34

(57) ABSTRACT

The invention provides deuterium-enriched thiazolidine-2,4-dione compounds (i.e., deuterium-enriched glitazone compounds), enantiopure forms of deuterium-enriched glitazone compounds, pharmaceutical compositions, and methods of treating medical disorders, such as a metabolic disorder, neurological disorder, cancer, or other disorder using deuterium-enriched glitazone compounds, which are preferably in enantiopure form.

### CROSS-REFERENCE TO RELATED APPLICATIONS

This application is a continuation of U.S. patent application Ser. No. 16/541,337, filed Aug. 15, 2019, which is also a continuation of U.S. patent application Ser. No. 15/705,544, filed Sep. 15, 2017, which is a continuation of International (PCT) Patent Application Serial No. PCT/US2016/023008, filed Mar. 18, 2016, which claims the benefit of and priority to U.S. Provisional Patent Application Ser. No. 62/135,995, filed Mar. 20, 2015, the contents of each of which are hereby incorporated by reference in their entirety.

### FIELD OF THE INVENTION

The invention provides deuterium-enriched thiazolidine-2,4-dione compounds (i.e., deuterium-enriched glitazone compounds), enantiopure forms of deuterium-enriched glitazone compounds, pharmaceutical compositions, and methods of treating medical disorders, such as a metabolic disorder, neurological disorder, cancer, or other disorder using deuterium-enriched glitazone compounds, which are preferably in enantiopure form.

### BACKGROUND

Peroxisome proliferator-activated receptors (PPARs) are a group of nuclear receptor proteins that function as transcription factors regulating the expression of genes. There are three subtypes of these receptors, PPAR alpha, beta, and gamma. PPARs mainly regulate the expression of genes involved in the regulation of lipid and carbohydrate metabolism. These receptors are also involved in the regulation of inflammatory processes, reproduction, carcinogenesis, and other physiological processes in the body. Treatment of a variety of medical disorders (e.g., Alzheimer's disease, cancer, and chronic obstructive pulmonary disease) has been linked to modulating the activity (e.g., activation) of certain PPARs.

Therapeutics that modulate PPARs have been commercialized for treating medical disorders, such as metabolic disorders. One such example is pioglitazone hydrochloride, which has been approved by the United States Food and Drug Administration as an adjunct to diet and exercise to improve glycemic control in adults with type 2 diabetes mellitus in multiple clinical settings. Pioglitazone hydrochloride is marketed under the registered trademark ACTOS© and the prescribing information for ACTOS© explains that pioglitazone is an agonist of PPAR gamma. The commercialized form of pioglitazone hydrochloride is a racemic mixture and adverse side effects have been reported in patients receiving this therapeutic, including, for example, edema and increased incidence of bone fracture.

Pioglitazone and other thiazolidinediones have been shown to have anti-inflammatory activity, part of which seems to be mediated by a mechanism not involving PPARs (Curr Drug Targets Inflamm Allergy 2002, 1(3):243-248). Recently, thiazolidinediones have also been shown to bind mitochondrial membrane proteins, including the mitochondrial target of thiazolidinedione (mTOT), and the thiazolidinediones may modulate mitochondrial metabolism through this direct binding. See, for example, PLoS One. 2013; 8(5): e61551; PNAS 2013, 110(14), 5422-5427; Am J Physiol Endocrinol Metab 2004, 286, E252-260.

Due to the increasing number of patients suffering from disorders such as those mentioned above, and the limitations of existing therapies, such as adverse side effects, there is a need for new therapeutic agents for treating medical disorders in which modulation of PPAR, anti-inflammatory, and/or mTOT activity are predicted to be beneficial. The present invention addresses these needs and provides other related advantages.

### SUMMARY OF THE INVENTION

The invention provides deuterium-enriched glitazone compounds, enantiopure forms of deuterium-enriched glitazone compounds, pharmaceutical compositions, and methods of treating medical disorders, such as a metabolic disorder, neurological disorder, cancer, or other disorder using deuterium-enriched glitazone compounds. The deuterated glitazone compounds contain deuterium enrichment at the chiral center of the thiazolidine-2,4-dionyl ring and optionally in other locations in the compound. Further, the deuterium-enriched glitazone compounds are preferably provided in enantiomerically pure form. Enantiomerically pure, deuterium-enriched glitazone compounds are contemplated to provide a better therapeutic agent than non-deuterated glitazone compounds and/or racemic mixtures of deuterium-enriched glitazone compounds.

Accordingly, one aspect of the invention provides a deuterium-enriched compound of Formula I for use in the therapeutic methods and pharmaceutical compositions described herein. Desirably, the deuterium-enriched compound of Formula I has a stereochemical purity of at least 75% enantiomeric excess. Formula I is represented by.

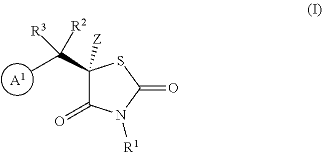

or a pharmaceutically acceptable salt thereof, wherein the variables are as defined in the detailed description. Additional generic formulae and specific deuterium-enriched compounds for use in the therapeutic methods and pharmaceutical compositions are described in the detailed description.

The deuterium-enriched compounds are particularly useful in the treatment of medical disorders. Exemplary medical disorders include, for example, metabolic disorders, neurological disorders, and cancer. Accordingly, another aspect of the invention provides a method of treating a medical disorder in a patient. The method comprises administering to a patient in need thereof a therapeutically effective amount of a compound described herein, such as deuterium-enriched compound of Formula I, to treat the disorder. In certain embodiments, the deuterium-enriched compound is a compound of Formula I-A, I-B, I-C, I-D, I-E, I-F, I-G, I-H, or I-I. In certain embodiments, the compound is administered orally.

Another aspect of the invention provides a method of treating a medical disorder in a patient. The method comprises orally administering to a patient in need thereof a therapeutically effective amount of a compound of Formula I-J to treat the disorder.

### DETAILED DESCRIPTION

The invention provides deuterium-enriched glitazone compounds, enantiopure forms of deuterium-enriched glitazone compounds, pharmaceutical compositions, and methods of treating medical disorders, such as a metabolic disorder, neurological disorder, cancer, or other disorder using deuterium-enriched glitazone compounds, which are preferably in enantiopure form. Deuterium-enriched refers to the feature that the compound has a quantity of deuterium that is greater than in naturally occurring compounds or synthetic compounds prepared from substrates having the naturally occurring distribution of isotopes. The threshold amount of deuterium enrichment is specified in certain instances in this disclosure, and all percentages given for the amount of deuterium present are mole percentages.

Deuterium (²H) is a stable, non-radioactive isotope of ¹H hydrogen and has an atomic weight of 2.014. Hydrogen naturally occurs as a mixture of the isotopes ¹H hydrogen (i.e., protium), deuterium (²H), and tritium (H). The natural abundance of deuterium is 0.015%. One of ordinary skill in the art recognizes that in all chemical compounds with an H atom, the H atom actually represents a mixture of ¹H hydrogen, deuterium (²H), and tritium (H), where about 0.015% is deuterium. Thus, compounds with a level of deuterium that has been enriched to be greater than its natural abundance of 0.015% are considered unnatural and, as a result, novel over their non-enriched counterparts.

Deuterium-enriched glitazone compounds described herein contain deuterium enrichment at the chiral center of the thiazolidine-2,4-dionyl ring and optionally in other locations in the compound. Deuterium-enrichment at the chiral center reduces the rate at which the two enantiomers may interconvert. Preferably, the deuterium-enriched glitazone compounds described herein are in enantiomerically pure form in the therapeutic methods and compositions. Enantiomerically pure, deuterium-enriched glitazone compounds are contemplated to provide a better therapeutic agent than non-deuterated glitazone compounds and/or racemic mixtures of non-deuterated glitazone compounds.

Exemplary compositions and methods of the present invention are described in more detail in the following sections: I. Deuterium-enriched Glitazone Compounds; II. Therapeutic Applications; III. Dosing Considerations and Combination Therapy, and IV. Pharmaceutical Compositions. Aspects of the invention described in one particular section are not to be limited to any particular section.

### I. Deuterium-Enriched Glitazone Compounds

One aspect of the invention provides deuterium-enriched glitazone compounds. Such compounds may be used in the therapeutic methods and pharmaceutical compositions described herein. The deuterium-enriched glitazone compounds are provided in high enantiomeric purity in order to maximize therapeutic benefit, such as maximize potency per dose of therapeutic agent and minimize adverse side effects. In one embodiment, the deuterium-enriched compound is a family of deuterium-enriched compounds represented by Formula I:

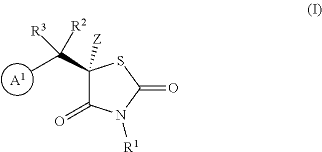

or a pharmaceutically acceptable salt thereof, wherein:
Z is H or D, provided that the abundance of deuterium in Z is at least 30%;
A¹ is one of the following

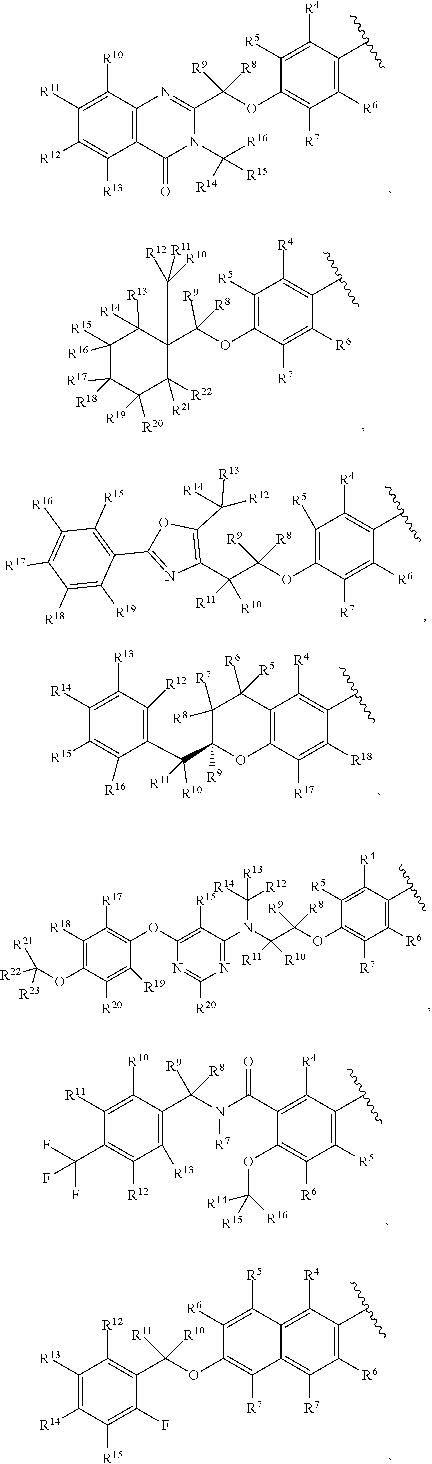

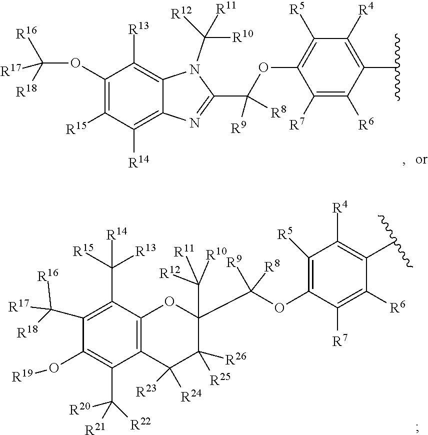

R¹, R², R³, R⁴, R⁵, R⁶, R⁷, R⁸, R⁹, R¹, R¹¹, R¹², R¹³, R¹⁴, R¹⁵, R¹⁶, R¹⁷, R¹⁸, R¹⁹, R²⁰, R²¹, R²², R²³, R²⁴, R²⁵, and R²⁶ are independently H or D; and the compound has a stereochemical purity of at least 75% enantiomeric excess at the carbon atom bearing variable Z.

In certain embodiments, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, and R²⁶ are H. In certain embodiments, the abundance of deuterium in Z is at least 80%, 90% or 95%. More specific embodiments of Formula I are provided below.

Another aspect of the invention provides a family of deuterium-enriched compounds represented by Formula II:

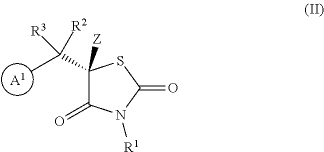

or a pharmaceutically acceptable salt thereof, wherein:
Z is H or D, provided that the abundance of deuterium in Z is at least 30%;
A¹ is one of the following:

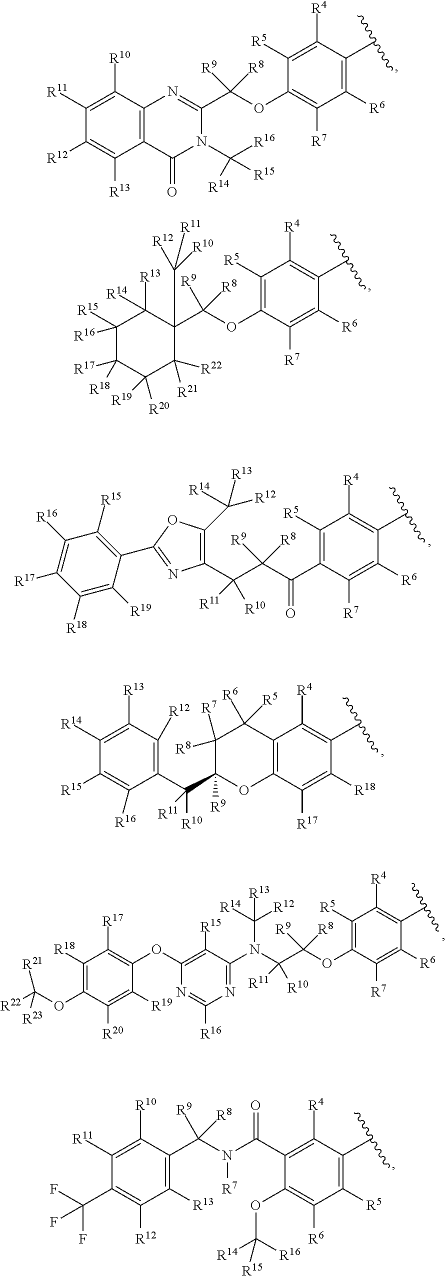

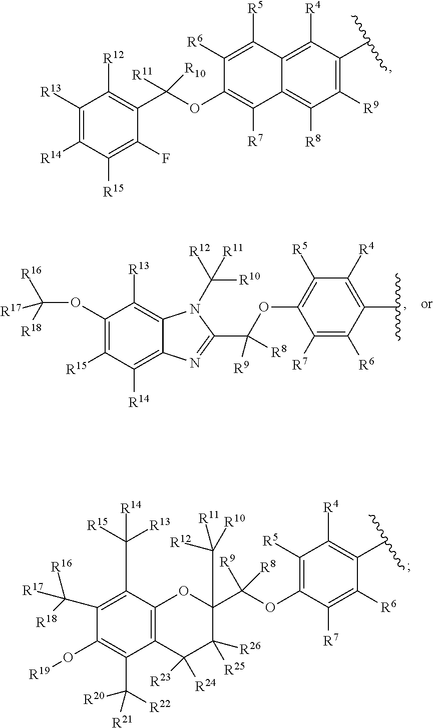

R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, and R²⁶ are independently H or D; and the compound has a stereochemical purity of at least 75% enantiomeric excess at the carbon atom bearing variable Z.

In certain embodiments, R¹, R², R³, R⁴, R⁵, R⁶, R⁷, R⁸, R⁹, R¹⁰, R¹¹, R¹², R¹³, R¹⁴, R, R¹⁶, R¹⁷, R¹⁸, R¹⁹, R²⁰, R²¹, R²², R²³, R²⁴, R²⁵, and R²⁶ are H. In certain embodiments, the abundance of deuterium in Z is at least 80%, 90% or 95%. More specific embodiments of Formula II are provided below.

### Deuterium-Enriched Balaglitazone

One collection of deuterium-enriched compounds is represented by Formula I-A having a stereochemical purity of at least 75% enantiomeric excess:

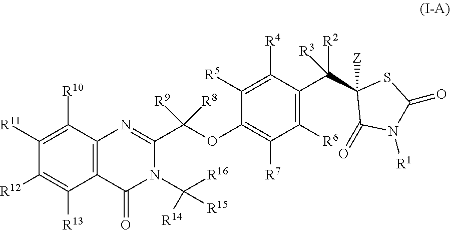

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, and R¹⁶ are independently H or D.

In certain embodiments, R² and R³ are H. In certain embodiments, R⁸ and R⁹ are H. In certain embodiments, R⁴, R⁵, R⁶, and R⁷ are H. In certain embodiments, R¹⁰, R¹¹, R¹², and R¹³ are H. In certain embodiments, R¹⁴, R¹⁵, and R¹⁶ are H. In certain other embodiments, R², R³, R⁸, and R⁹ are H.

In certain embodiments, R², R³, R⁸, R⁹, R¹⁴, R⁵, and R¹⁶ are H. In certain embodiments, R⁴, R⁵, R⁶, R⁷, R⁸, and R⁹ are H. In certain embodiments, R⁴, R⁵, R⁶, R⁷, R¹⁰, R¹¹, R¹², and R¹³ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, and R⁸ are H.

The description above describes multiple embodiments relating to compounds of Formula I-A. The patent application specifically contemplates all combinations of the embodiments. For example, the invention contemplates a compound of Formula I-A wherein R², R³, R⁴, R⁵, R⁶, R⁷, R⁸, and R⁹ are H.

Another collection of deuterium-enriched compounds is represented by Formula I-A1 having a stereochemical purity of at least 75% enantiomeric excess:

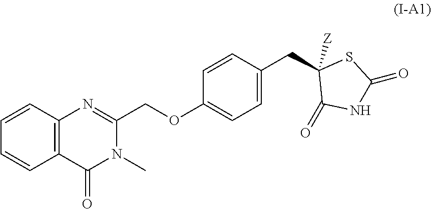

or a pharmaceutically acceptable salt thereof, wherein Z is H or D, provided that the abundance of deuterium in Z is at least 30%.

The compounds of Formula I-A and Formula I-A1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula I-A and Formula I-A1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 1 and 2 below.

TABLE 1

| Compound | |
|---|---|
| No. | Structure |
| 1 | 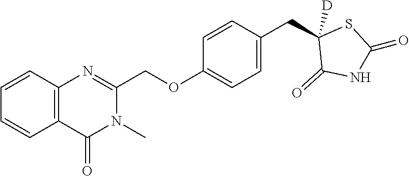 [SMILES: [2H][C@]1(Cc2ccc(OCc3nc4ccccc4c(=O)n3C)cc2)SC(=O)NC1=O] |
| | or a pharmaceutically acceptable |
| | salt thereof, each having a stereochemical purity of at least 60% |
| | enantiomeric excess. |
| 2 | 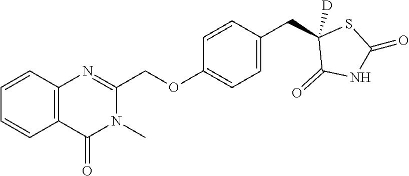 [SMILES: [2H][C@]1(Cc2ccc(OCc3nc4ccccc4c(=O)n3C)cc2)SC(=O)NC1=O] |
| | having a stereochemical purity of at |
| | least 60% enantiomeric excess. |
| 3 | 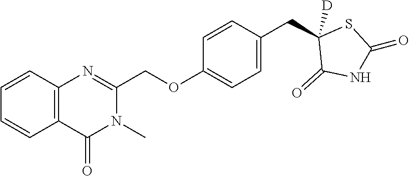 [SMILES: [2H][C@]1(Cc2ccc(OCc3nc4ccccc4c(=O)n3C)cc2)SC(=O)NC1=O] |
| | or a pharmaceutically acceptable |
| | salt thereof, each having a stereochemical purity of at least 75% |
| | enantiomeric excess. |
| 4 | 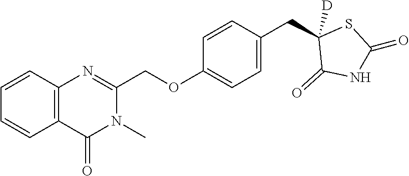 [SMILES: [2H][C@]1(Cc2ccc(OCc3nc4ccccc4c(=O)n3C)cc2)SC(=O)NC1=O] |
| | having a stereochemical purity of at |
| | least 75% enantiomeric excess. |
| 5 | 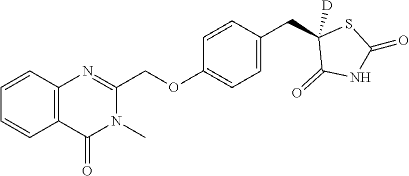 [SMILES: [2H][C@]1(Cc2ccc(OCc3nc4ccccc4c(=O)n3C)cc2)SC(=O)NC1=O] |
| | or a pharmaceutically acceptable |
| | salt thereof, each having a stereochemical purity of at least 90% |
| | enantiomeric excess. |
| 6 | 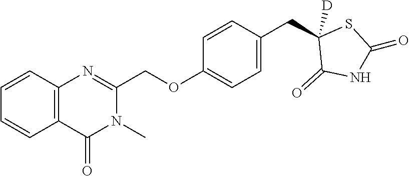 [SMILES: [2H][C@]1(Cc2ccc(OCc3nc4ccccc4c(=O)n3C)cc2)SC(=O)NC1=O] |
| | having a stereochemical purity of at |
| | least 90% enantiomeric excess. |
| 7 | 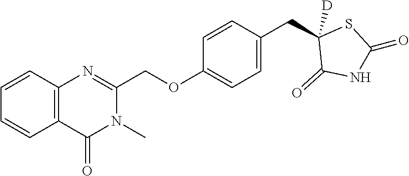 [SMILES: [2H][C@]1(Cc2ccc(OCc3nc4ccccc4c(=O)n3C)cc2)SC(=O)NC1=O] |
| | or a pharmaceutically acceptable |
| | salt thereof, each having a stereochemical purity of at least 95% |
| | enantiomeric excess. |
| 8 | 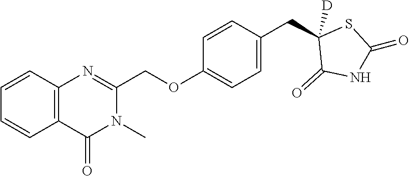 [SMILES: [2H][C@]1(Cc2ccc(OCc3nc4ccccc4c(=O)n3C)cc2)SC(=O)NC1=O] |
| | having a stereochemical purity of at |
| | least 95% enantiomeric excess. |

TABLE 2

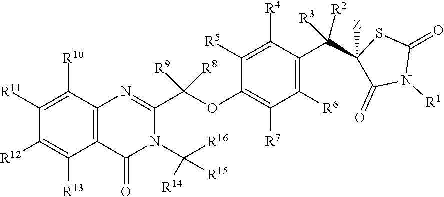

| Compound | |
|---|---|
| No. | Variable Definition |
| 1 | Z = D; R¹ = H; R²-R⁹ are D; and R¹⁰-R¹⁶ are H |
| 2 | Z = D; R¹ = H; R² and R³ are D; and R⁴-R¹⁶ are H |
| 3 | Z = D; R¹-R³ are H; R⁴-R⁷ are D; and R⁸-R¹⁶ are H |
| 4 | Z = D; R¹-R⁷ are H; R⁸-R¹³ are D; and R¹⁴-R¹⁶ are H |
| 5 | Z = D; R¹-R⁹ are H; R¹⁰-R¹³ are D; and R¹⁴-R¹⁶ are H |
| 6 | Z = D; R¹ = H; R² and R³ are D; R⁴-R⁷ are H; R⁸ and R⁹ are D; and R¹⁰-R¹⁶ are H |

Another embodiment provides a compound in Table 2 wherein the compound has an enantiomeric excess of at least 60%, 70%, 75%, 80%, 85%, 90%, 95%, or 98%.

Another collection of deuterium-enriched compounds is represented by Formula II-A having a stereochemical purity of at least 75% enantiomeric excess:

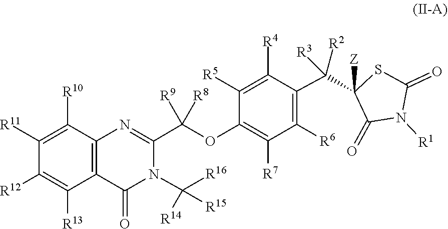

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, and R¹⁶ are independently H or D.

In certain embodiments, R² and R³ are H. In certain embodiments, R⁸ and R⁹ are H. In certain embodiments, R⁴, R⁵, R⁶, and R⁷ are H. In certain embodiments, R¹⁰, R¹¹, R¹², and R¹³ are H. In certain embodiments, R¹⁴, R¹⁵, and R¹⁶ are H. In certain other embodiments, R², R³, R⁸, and R⁹ are H.

In certain embodiments, R², R³, R⁸, R⁹, R¹⁴, R¹⁵, and R¹⁶ are H. In certain embodiments, R⁴, R⁵, R⁶, R⁷, R⁸, and R⁹ are H. In certain embodiments, R⁴, R⁵, R⁶, R⁷, R¹⁰, R¹¹, R¹², and R¹³ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, and R⁸ are H.

The description above describes multiple embodiments relating to compounds of Formula II-A. The patent application specifically contemplates all combinations of the embodiments. For example, the invention contemplates a compound of Formula II-A wherein R², R³, R⁴, R⁵, R⁶, R⁷, R⁸, and R⁹ are H.

Another collection of deuterium-enriched compounds is represented by Formula II-A1 having a stereochemical purity of at least 75% enantiomeric excess:

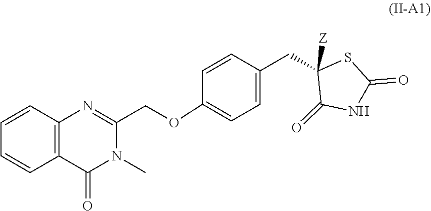

or a pharmaceutically acceptable salt thereof, wherein Z is H or D, provided that the abundance of deuterium in Z is at least 30%.

The compounds of Formula II-A and Formula II-A1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula II-A and Formula II-A1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 3 and 4 below.

TABLE 3

| Compound | |
|---|---|
| No. | Structure |
| 1 | 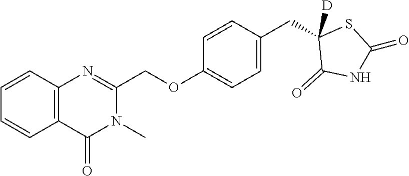 [SMILES: [2H][C@@]1(Cc2ccc(OCc3nc4ccccc4c(=O)n3C)cc2)SC(=O)NC1=O] |
| | or a pharmaceutically acceptable |
| | salt thereof, each having a stereochemical purity of at least 60% |
| | enantiomeric excess. |
| 2 | 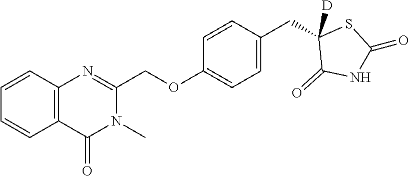 [SMILES: [2H][C@@]1(Cc2ccc(OCc3nc4ccccc4c(=O)n3C)cc2)SC(=O)NC1=O] |
| | having a stereochemical purity of at |
| | least 60% enantiomeric excess. |
| 3 | 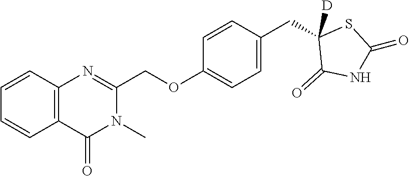 [SMILES: [2H][C@@]1(Cc2ccc(OCc3nc4ccccc4c(=O)n3C)cc2)SC(=O)NC1=O] |
| | or a pharmaceutically acceptable |
| | salt thereof, each having a stereochemical purity of at least 75% |
| | enantiomeric excess. |
| 4 | 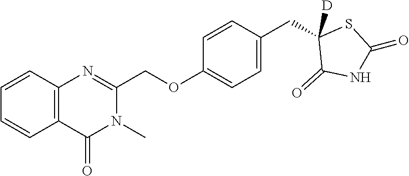 [SMILES: [2H][C@@]1(Cc2ccc(OCc3nc4ccccc4c(=O)n3C)cc2)SC(=O)NC1=O] |
| | having a stereochemical purity of at |
| | least 75% enantiomeric excess. |
| 5 | 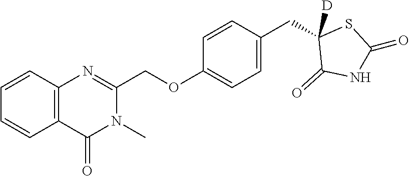 [SMILES: [2H][C@@]1(Cc2ccc(OCc3nc4ccccc4c(=O)n3C)cc2)SC(=O)NC1=O] |
| | or a pharmaceutically acceptable |
| | salt thereof, each having a stereochemical purity of at least 90% |
| | enantiomeric excess. |
| 6 | 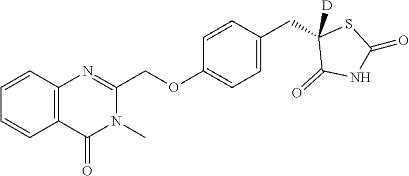 [SMILES: [2H][C@@]1(Cc2ccc(OCc3nc4ccccc4c(=O)n3C)cc2)SC(=O)NC1=O] |
| | having a stereochemical purity of at |
| | least 90% enantiomeric excess. |
| 7 | 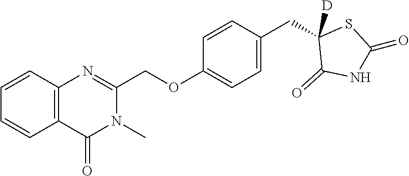 [SMILES: [2H][C@@]1(Cc2ccc(OCc3nc4ccccc4c(=O)n3C)cc2)SC(=O)NC1=O] |
| | or a pharmaceutically acceptable |
| | salt thereof, each having a stereochemical purity of at least 95% |
| | enantiomeric excess. |
| 8 | 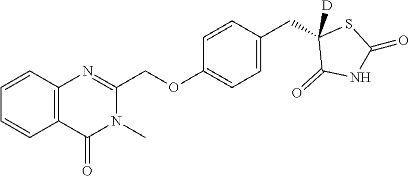 [SMILES: [2H][C@@]1(Cc2ccc(OCc3nc4ccccc4c(=O)n3C)cc2)SC(=O)NC1=O] |
| | having a stereochemical purity of at |
| | least 95% enantiomeric excess. |

TABLE 4

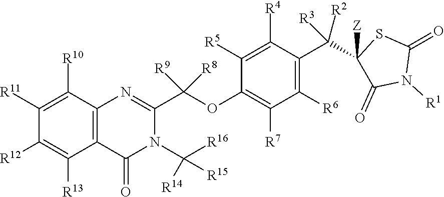

| Compound | |
|---|---|
| No. | Variable Definition |
| 1 | Z = D; R¹ = H; R²-R⁹ are D; and R¹⁰-R¹⁶ are H |
| 2 | Z = D; R¹ = H; R² and R³ are D; and R⁴-R¹⁶ are H |
| 3 | Z = D; R¹-R³ are H; R⁴-R⁷ are D; and R⁸-R¹⁶ are H |
| 4 | Z = D; R¹-R⁷ are H; R⁸-R¹³ are D; and R¹⁴-R¹⁶ are H |
| 5 | Z = D; R¹-R⁹ are H; R¹⁰-R¹³ are D; and R¹⁴-R¹⁶ are H |
| 6 | Z = D; R¹ = H; R² and R³ are D; R⁴-R⁷ are H; R⁸ and R⁹ are D; and R¹⁰-R¹⁶ are H |

Another embodiment provides a compound in Table 4 wherein the compound has an enantiomeric excess of at least 60%, 70%, 75%, 80%, 85%, 90%, 95%, or 98%.

### Deuterium-Enriched Ciglitazone

Another collection of deuterium-enriched compounds is represented by Formula I-B having a stereochemical purity of at least 75% enantiomeric excess:

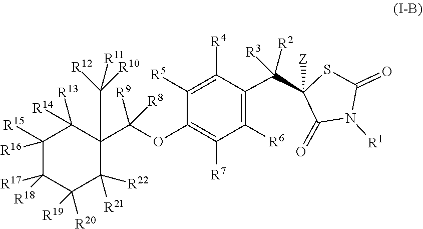

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹, R¹⁶, R¹⁷, R¹⁸, R¹⁹, R²⁰, R²¹, and R²² are independently H or D.

In certain embodiments, R² and R³ are H. In certain embodiments, R⁸ and R⁹ are H. In certain embodiments, R⁴, R⁵, R⁶, and R⁷ are H. In certain embodiments, R⁸, R⁹, R¹⁰, R¹¹, and R¹² are H. In certain embodiments, R¹³, R¹⁴, R⁵, R¹⁶, R¹⁷, R¹⁸, R¹⁹, R²⁰, R²¹, and R²² are H. In certain other embodiments, R², R³, R⁸, and R⁹ are H.

In certain embodiments, R², R³, R⁸, R⁹, R¹⁰, R¹¹, and R¹² are H. In certain embodiments, R⁴, R⁵, R⁶, R⁷, R⁸, and R⁹ are H. In certain embodiments, R⁴, R⁵, R⁶, R⁷, R¹³, R¹⁴, R¹⁵, R¹⁶, R¹⁷, R¹⁸, R¹⁹, R²⁰, R²¹, and R²² are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, R⁸ and R⁹ are H.

The description above describes multiple embodiments relating to compounds of Formula I-B. The patent application specifically contemplates all combinations of the embodiments. For example, the invention contemplates a compound of Formula I-B wherein R², R³, R⁴, R⁵, R⁶, R⁷, R⁸, R⁹, R¹³, R¹⁴, R¹⁵, R¹⁶, R¹⁷, R¹⁸, R¹⁹, R²⁰, R²¹, and R²² are H.

Another collection of deuterium-enriched compounds is represented by Formula I-B1 having a stereochemical purity of at least 75% enantiomeric excess:

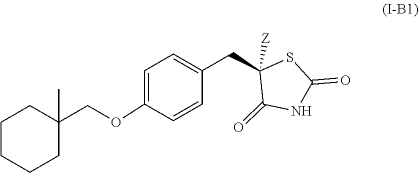

or a pharmaceutically acceptable salt thereof, wherein Z is H or D, provided that the abundance of deuterium in Z is at least 30%.

The compounds of Formula I-B and Formula I-B1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula I-B and Formula I-B1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 5 and 6 below.

TABLE 5

| Compound | |
|---|---|
| No. | Structure |
| 1 | 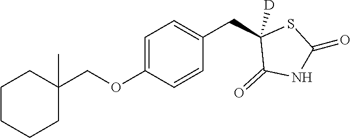 [SMILES: [2H][C@]1(Cc2ccc(OCC3(C)CCCCC3)cc2)SC(=O)NC1=O] |
| | or a pharmaceutically acceptable salt |
| | thereof, each having a stereochemical purity of at least 60% enantiomeric |
| | excess. |
| 2 | 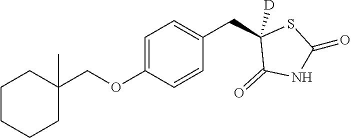 [SMILES: [2H][C@]1(Cc2ccc(OCC3(C)CCCCC3)cc2)SC(=O)NC1=O] |
| | having a stereochemical purity of at least |
| | 60% enantiomeric excess. |
| 3 | 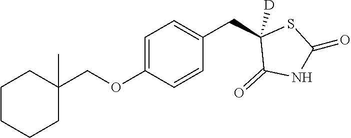 [SMILES: [2H][C@]1(Cc2ccc(OCC3(C)CCCCC3)cc2)SC(=O)NC1=O] |
| | or a pharmaceutically acceptable salt |
| | thereof, each having a stereochemical purity of at least 75% enantiomeric |
| | excess. |
| 4 | 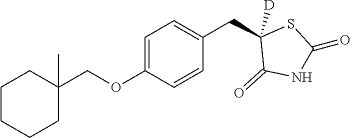 [SMILES: [2H][C@]1(Cc2ccc(OCC3(C)CCCCC3)cc2)SC(=O)NC1=O] |
| | having a stereochemical purity of at least |
| | 75% enantiomeric excess. |
| 5 | 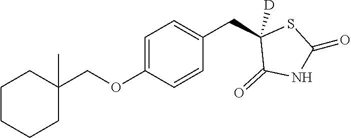 [SMILES: [2H][C@]1(Cc2ccc(OCC3(C)CCCCC3)cc2)SC(=O)NC1=O] |
| | or a pharmaceutically acceptable salt |
| | thereof, each having a stereochemical purity of at least 90% enantiomeric |
| | excess. |
| 6 | 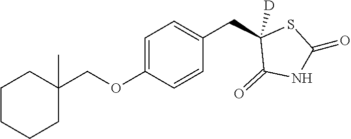 [SMILES: [2H][C@]1(Cc2ccc(OCC3(C)CCCCC3)cc2)SC(=O)NC1=O] |
| | having a stereochemical purity of at least |
| | 90% enantiomeric excess. |
| 7 | 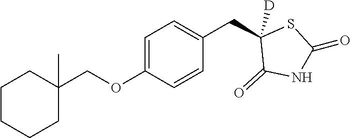 [SMILES: [2H][C@]1(Cc2ccc(OCC3(C)CCCCC3)cc2)SC(=O)NC1=O] |
| | or a pharmaceutically acceptable salt |
| | thereof, each having a stereochemical purity of at least 95% enantiomeric |
| | excess. |
| 8 | 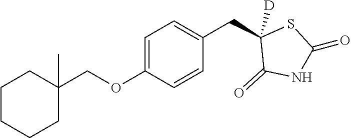 [SMILES: [2H][C@]1(Cc2ccc(OCC3(C)CCCCC3)cc2)SC(=O)NC1=O] |
| | having a stereochemical purity of at least |
| | 95% enantiomeric excess. |

TABLE 6

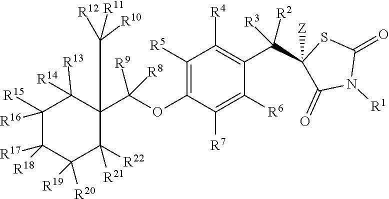

| Compound | |
|---|---|
| No. | Variable Definition |
| 1 | Z = D; R¹ = H; R²-R⁹ are D; and R¹⁰-R²² are H |
| 2 | Z = D; R¹ = H; R² and R³ are D; and R⁴-R²² are H |
| 3 | Z = D; R¹-R³ are H; R⁴-R⁷ are D; and R⁸-R²² are H |
| 4 | Z = D; R¹-R⁷ are H; R⁸-R¹² are D; and R¹³-R²² are H |
| 5 | Z = D; R¹-R⁹ are H; R¹⁰-R¹² are D; and R¹³-R¹² are H |
| 6 | Z = D; R¹ = H; R² and R³ are D; R⁴-R⁷ are H; R⁸-R⁹ are D; R¹⁰-R¹² are H; |
| | R¹³ and R¹⁴ are D; R¹⁵-R²⁰ are H; and R²¹ and R²² are D |

Another embodiment provides a compound in Table 6 wherein the compound has an enantiomeric excess of at least 60%, 70%, 75%, 80%, 85%, 90%, 95%, or 98%.

Another collection of deuterium-enriched compounds is represented by Formula II-B having a stereochemical purity of at least 75% enantiomeric excess:

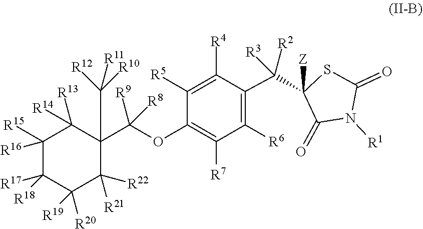

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹ and R²² are independently H or D.

In certain embodiments, R² and R³ are H. In certain embodiments, R⁸ and R⁹ are H. In certain embodiments, R⁴, R⁵, R⁶, and R⁷ are H. In certain embodiments, R⁸, R⁹, R¹⁰, R¹¹, and R¹² are H. In certain embodiments, R¹³, R¹⁴, R⁵, R¹⁶, R¹⁷, R¹⁸, R¹⁹, R²⁰, R²¹, and R²² are H. In certain other embodiments, R², R³, R⁸, and R⁹ are H.

In certain embodiments, R², R³, R⁸, R⁹, R¹⁰, R¹¹, and R¹² are H. In certain embodiments, R⁴, R⁵, R⁶, R⁷, R⁸, and R⁹ are H. In certain embodiments, R⁴, R⁵, R⁶, R⁷, R¹³, R¹⁴, R¹⁵, R¹⁶, R¹⁷, R¹⁸, R¹⁹, R²⁰, R²¹, and R²² are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, R⁸ and R⁹ are H.

The description above describes multiple embodiments relating to compounds of Formula II-B. The patent application specifically contemplates all combinations of the embodiments. For example, the invention contemplates a compound of Formula II-B wherein R², R³, R⁴, R⁵, R⁶, R⁷, R⁸, R⁹, R¹³, R¹⁴, R⁵, R¹⁶, R¹⁷, R¹⁸, R¹⁹, R²⁰, R²¹, and R²² are H.

Another collection of deuterium-enriched compounds is represented by Formula II-B1 having a stereochemical purity of at least 75% enantiomeric excess:

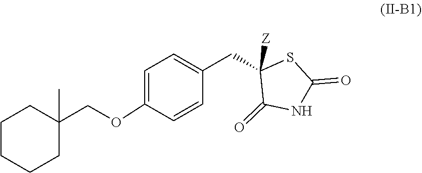

or a pharmaceutically acceptable salt thereof, wherein Z is H or D, provided that the abundance of deuterium in Z is at least 30%.

The compounds of Formula II-B and Formula II-B1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula II-B and Formula II-B1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 7 and 8 below.

TABLE 7

| Compound | |
|---|---|
| No. | Structure |
| 1 | 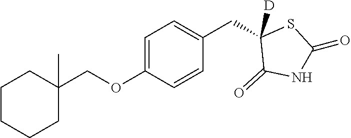 [SMILES: [2H][C@@]1(Cc2ccc(OCC3(C)CCCCC3)cc2)SC(=O)NC1=O] |
| | or a pharmaceutically acceptable salt |
| | thereof, each having a stereochemical purity of at least 60% enantiomeric |
| | excess. |
| 2 | 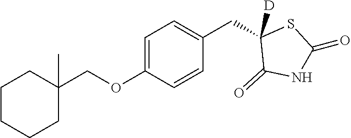 [SMILES: [2H][C@@]1(Cc2ccc(OCC3(C)CCCCC3)cc2)SC(=O)NC1=O] |
| | having a stereochemical purity of at least |
| | 60% enantiomeric excess. |
| 3 | 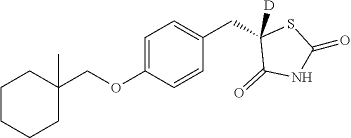 [SMILES: [2H][C@@]1(Cc2ccc(OCC3(C)CCCCC3)cc2)SC(=O)NC1=O] |
| | or a pharmaceutically acceptable salt |
| | thereof, each having a stereochemical purity of at least 75% enantiomeric |
| | excess. |
| 4 | 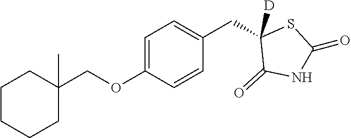 [SMILES: [2H][C@@]1(Cc2ccc(OCC3(C)CCCCC3)cc2)SC(=O)NC1=O] |
| | having a stereochemical purity of at least |
| | 75% enantiomeric excess. |
| 5 | 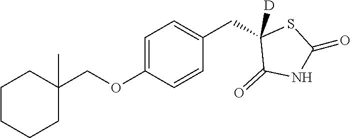 [SMILES: [2H][C@@]1(Cc2ccc(OCC3(C)CCCCC3)cc2)SC(=O)NC1=O] |
| | or a pharmaceutically acceptable salt |
| | thereof, each having a stereochemical purity of at least 90% enantiomeric |
| | excess. |
| 6 | 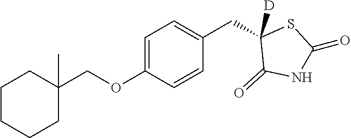 [SMILES: [2H][C@@]1(Cc2ccc(OCC3(C)CCCCC3)cc2)SC(=O)NC1=O] |
| | having a stereochemical purity of at least |
| | 90% enantiomeric excess. |
| 7 | 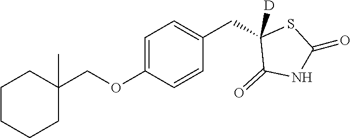 [SMILES: [2H][C@@]1(Cc2ccc(OCC3(C)CCCCC3)cc2)SC(=O)NC1=O] |
| | or a pharmaceutically acceptable salt |
| | thereof, each having a stereochemical purity of at least 95% enantiomeric |
| | excess. |
| 8 | 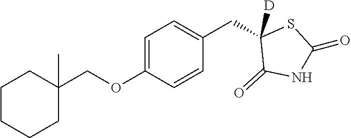 [SMILES: [2H][C@@]1(Cc2ccc(OCC3(C)CCCCC3)cc2)SC(=O)NC1=O] |
| | having a stereochemical purity of at least |
| | 95% enantiomeric excess. |

TABLE 8

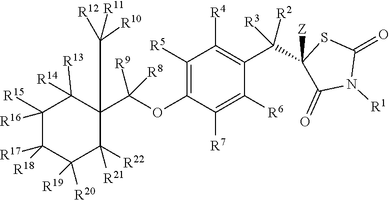

| Compound | |
|---|---|
| No. | Variable Definition |
| 1 | Z = D; R¹ = H; R²-R⁹ are D; and R¹⁰-R²² are H |
| 2 | Z = D; R¹ = H; R² and R³ are D; and R⁴-R²² are H |
| 3 | Z = D; R¹-R³ are H; R⁴-R⁷ are D; and R⁸-R²² are H |
| 4 | Z = D; R¹-R⁷ are H; R⁸-R¹² are D; and R¹³-R²² are H |
| 5 | Z = D; R¹-R⁹ are H; R¹⁰-R¹² are D; and R¹³-R¹² are H |
| 6 | Z = D; R¹ = H; R² and R³ are D; R⁴-R⁷ are H; R⁸-R⁹ are D; R¹⁰-R¹² are H; |
| | R¹³ and R¹⁴ are D; R¹⁵-R²⁰ are H; and R²¹ and R²² are D |

Another embodiment provides a compound in Table 8 wherein the compound has an enantiomeric excess of at least 60%, 70%, 75%, 80%, 85%, 90%, 95%, or 98%.

### Deuterium-Enriched Darglitazone

Another collection of deuterium-enriched compounds is represented by Formula I-C having a stereochemical purity of at least 75% enantiomeric excess:

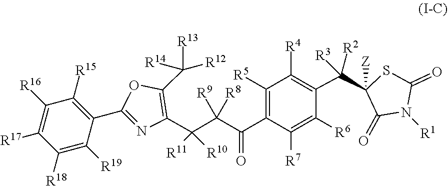

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, and R¹⁹ are independently H or D.

In certain embodiments, R² and R³ are H. In certain embodiments, R⁸ and R⁹ are H. In certain embodiments, R⁴, R⁵, R⁶, and R⁷ are H. In certain embodiments, R⁸, R⁹, R¹⁰, and R¹¹ are H. In certain embodiments, R¹⁵, R¹⁶, R¹⁷, R¹⁸, and R¹⁹ are H. In certain other embodiments, R², R³, R⁸, and R⁹ are H.

In certain embodiments, R², R³, R⁸, R⁹, R¹⁰, and R¹¹ are H. In certain embodiments, R⁴, R⁵, R⁶, R⁷, R⁸, and R⁹ are H. In certain embodiments, R⁴, R⁵, R⁶, R, R¹³, R¹⁴, R¹⁵, R¹⁶, R¹⁷, R¹⁸, and R¹⁹ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, R⁸ and R⁹ are H.

The description above describes multiple embodiments relating to compounds of Formula I-C. The patent application specifically contemplates all combinations of the embodiments. For example, the invention contemplates a compound of Formula I-C wherein R², R³, R⁴, R⁵, R⁶, R⁷, R⁸, R⁹, R¹⁰, and R¹¹ are H.

Another collection of deuterium-enriched compounds is represented by Formula I-C1 having a stereochemical purity of at least 75% enantiomeric excess:

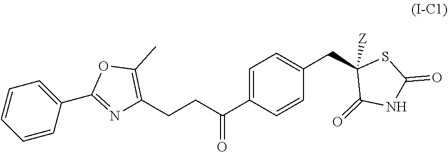

or a pharmaceutically acceptable salt thereof, wherein Z is H or D, provided that the abundance of deuterium in Z is at least 30%.

The compounds of Formula I-C and Formula I-C1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula I-C and Formula I-C1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 9 and 10 below.

TABLE 9

| Compound | |
|---|---|
| No. | Structure |
| 1 | 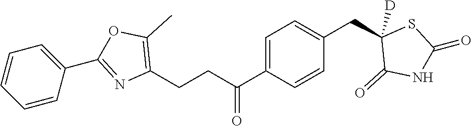 [SMILES: [2H][C@]1(Cc2ccc(C(=O)CCc3nc(-c4ccccc4)oc3C)cc2)SC(=O)NC1=O] |
| | or a pharmaceutically |
| | acceptable salt thereof, each having a stereochemical purity of at least 60% |
| | enantiomeric excess. |
| 2 | 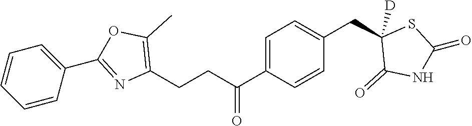 [SMILES: [2H][C@]1(Cc2ccc(C(=O)CCc3nc(-c4ccccc4)oc3C)cc2)SC(=O)NC1=O] |
| | having a stereochemical |
| | purity of at least 60% enantiomeric excess. |
| 3 | 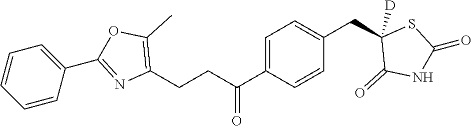 [SMILES: [2H][C@]1(Cc2ccc(C(=O)CCc3nc(-c4ccccc4)oc3C)cc2)SC(=O)NC1=O] |
| | or a pharmaceutically |
| | acceptable salt thereof, each having a stereochemical purity of at least 75% |
| | enantiomeric excess. |
| 4 | 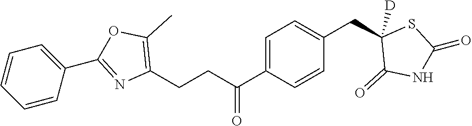 [SMILES: [2H][C@]1(Cc2ccc(C(=O)CCc3nc(-c4ccccc4)oc3C)cc2)SC(=O)NC1=O] |
| | having a stereochemical |
| | purity of at least 75% enantiomeric excess. |
| 5 | 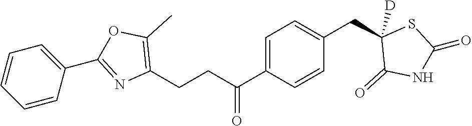 [SMILES: [2H][C@]1(Cc2ccc(C(=O)CCc3nc(-c4ccccc4)oc3C)cc2)SC(=O)NC1=O] |
| | or a pharmaceutically |
| | acceptable salt thereof, each having a stereochemical purity of at least 90% |
| | enantiomeric excess. |
| 6 | 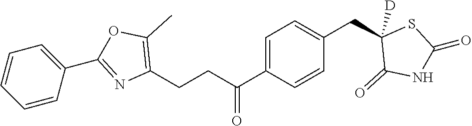 [SMILES: [2H][C@]1(Cc2ccc(C(=O)CCc3nc(-c4ccccc4)oc3C)cc2)SC(=O)NC1=O] |
| | having a stereochemical |
| | purity of at least 90% enantiomeric excess. |
| 7 | 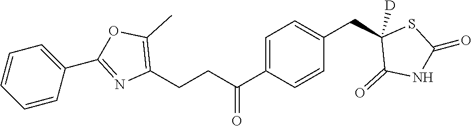 [SMILES: [2H][C@]1(Cc2ccc(C(=O)CCc3nc(-c4ccccc4)oc3C)cc2)SC(=O)NC1=O] |
| | or a pharmaceutically |
| | acceptable salt thereof, each having a stereochemical purity of at least 95% |
| | enantiomeric excess. |
| 8 | 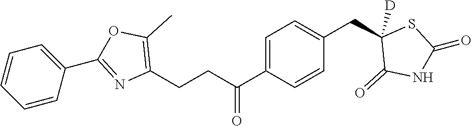 [SMILES: [2H][C@]1(Cc2ccc(C(=O)CCc3nc(-c4ccccc4)oc3C)cc2)SC(=O)NC1=O] |
| | having a stereochemical |
| | purity of at least 95% enantiomeric excess. |

TABLE 10

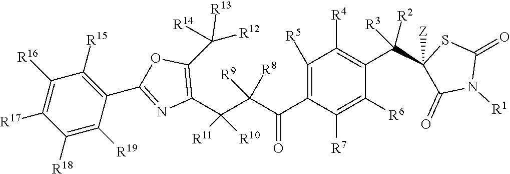

| Compound | |
|---|---|
| No. | Variable Definition |
| 1 | Z = D; R¹ = H; R²-R⁹ are D; and R¹⁰-R¹⁹ are H |
| 2 | Z = D; R¹ = H; R² and R³ are D; and R⁴-R¹⁹ are H |
| 3 | Z = D; R¹-R³ are H; R⁴-R⁷ are D; and R⁸-R¹⁹ are H |
| 4 | Z = D; R¹-R⁷ are H; R⁸-R¹¹ are D; and R¹²-R¹⁹ are H |
| 5 | Z = D; R¹-R⁹ are H; R¹⁰-R¹¹ are D; and R¹²-R¹⁹ are H |
| 6 | Z = D; R¹ = H; R² and R³ are D; R⁴-R⁷ are H; R⁸-R⁹ are D; R¹⁰-R¹⁴ are H; |
| | R¹⁵-R¹⁹ are D |

Another embodiment provides a compound in Table 10 wherein the compound has an enantiomeric excess of at least 60%, 70%, 75%, 80%, 85%, 90%, 95%, or 98%.

Another collection of deuterium-enriched compounds is represented by Formula II-C having a stereochemical purity of at least 75% enantiomeric excess:

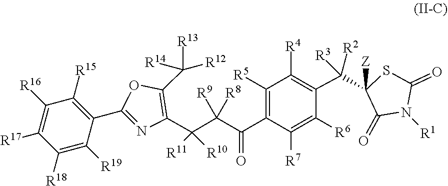

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, and R¹⁹ are independently H or D.

In certain embodiments, R² and R³ are H. In certain embodiments, R⁸ and R⁹ are H. In certain embodiments, R⁴, R⁵, R⁶, and R⁷ are H. In certain embodiments, R⁸, R⁹, R¹⁰, and R¹¹ are H. In certain embodiments, R¹⁵, R¹⁶, R¹⁷, R¹⁸, and R¹⁹ are H. In certain other embodiments, R², R³, R⁸, and R⁹ are H.

In certain embodiments, R², R³, R⁸, R⁹, R¹⁰, and R¹¹ are H. In certain embodiments, R⁴, R⁵, R⁶, R⁷, R⁸, and R⁹ are H. In certain embodiments, R⁴, R⁵, R⁶, R⁷, R¹², R¹³, R¹⁴, R¹⁵, R¹⁶, R¹⁷, R¹⁸, and R¹⁹ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, R⁸ and R⁹ are H.

The description above describes multiple embodiments relating to compounds of Formula II-C. The patent application specifically contemplates all combinations of the embodiments. For example, the invention contemplates a compound of Formula II-C wherein R², R³, R⁴, R⁵, R⁶, R⁷, R⁸, R⁹, R¹⁰, and R¹¹ are H.

Another collection of deuterium-enriched compounds is represented by Formula II-C1 having a stereochemical purity of at least 75% enantiomeric excess:

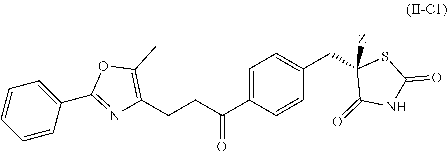

or a pharmaceutically acceptable salt thereof, wherein Z is H or D, provided that the abundance of deuterium in Z is at least 30%.

The compounds of Formula II-C and Formula II-C1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 10000.

In certain embodiments, the abundance of deuterium in Z is at least 600%. In certain other embodiments, the abundance of deuterium in Z is at least 7500 In yet other embodiments, the abundance of deuterium in Z is at least 9000.

The compounds of Formula II-C and Formula II-C1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 11 and 12 below.

TABLE 11

| Compound | |
|---|---|
| No. | Structure |
| 1 | 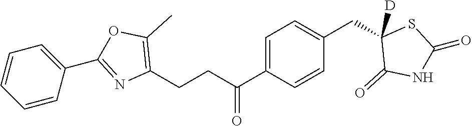 [SMILES: [2H][C@@]1(Cc2ccc(C(=O)CCc3nc(-c4ccccc4)oc3C)cc2)SC(=O)NC1=O] |
| | or a pharmaceutically |
| | acceptable salt thereof, each having a stereochemical purity of at least 60% |
| | enantiomeric excess. |
| 2 | 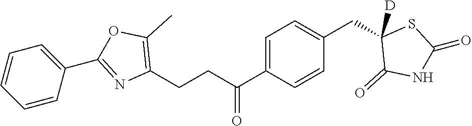 [SMILES: [2H][C@@]1(Cc2ccc(C(=O)CCc3nc(-c4ccccc4)oc3C)cc2)SC(=O)NC1=O] |
| | having a stereochemical |
| | purity of at least 60% enantiomeric excess. |
| 3 | 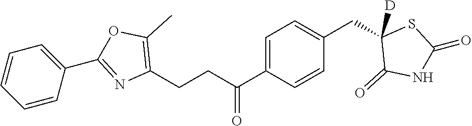 [SMILES: [2H][C@@]1(Cc2ccc(C(=O)CCc3nc(-c4ccccc4)oc3C)cc2)SC(=O)NC1=O] |
| | or a pharmaceutically |
| | acceptable salt thereof, each having a stereochemical purity of at least 75% |
| | enantiomeric excess. |
| 4 | 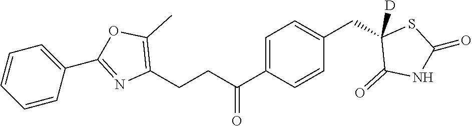 [SMILES: [2H][C@@]1(Cc2ccc(C(=O)CCc3nc(-c4ccccc4)oc3C)cc2)SC(=O)NC1=O] |
| | having a stereochemical |
| | purity of at least 75% enantiomeric excess. |
| 5 | 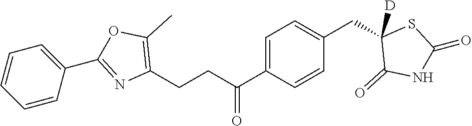 [SMILES: [2H][C@@]1(Cc2ccc(C(=O)CCc3nc(-c4ccccc4)oc3C)cc2)SC(=O)NC1=O] |
| | or a pharmaceutically |
| | acceptable salt thereof, each having a stereochemical purity of at least 90% |
| | enantiomeric excess. |
| 6 | 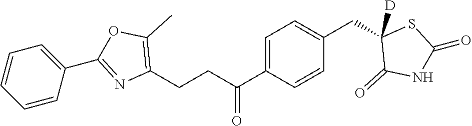 [SMILES: [2H][C@@]1(Cc2ccc(C(=O)CCc3nc(-c4ccccc4)oc3C)cc2)SC(=O)NC1=O] |
| | having a stereochemical |
| | purity of at least 90% enantiomeric excess. |
| 7 | 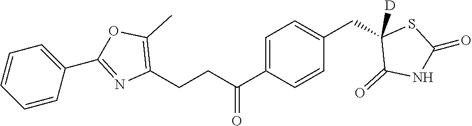 [SMILES: [2H][C@@]1(Cc2ccc(C(=O)CCc3nc(-c4ccccc4)oc3C)cc2)SC(=O)NC1=O] |
| | or a pharmaceutically |
| | acceptable salt thereof, each having a stereochemical purity of at least 95% |
| | enantiomeric excess. |
| 8 | 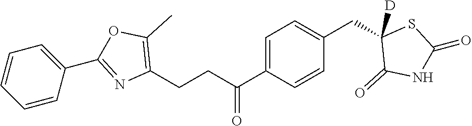 [SMILES: [2H][C@@]1(Cc2ccc(C(=O)CCc3nc(-c4ccccc4)oc3C)cc2)SC(=O)NC1=O] |
| | having a stereochemical |
| | purity of at least 95% enantiomeric excess. |

TABLE 12

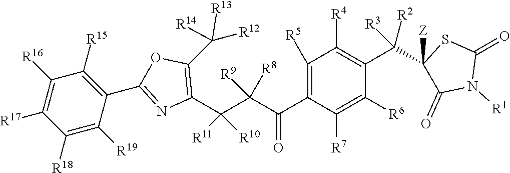

| Compound | |
|---|---|
| No. | Variable Definition |
| 1 | Z = D; R¹ = H; R²-R⁹ are D; and R¹⁰-R¹⁹ are H |
| 2 | Z = D; R¹ = H; R² and R³ are D; and R⁴-R¹⁹ are H |
| 3 | Z = D; R¹-R³ are H; R⁴-R⁷ are D; and R⁸-R¹⁹ are H |
| 4 | Z = D; R¹-R⁷ are H; R⁸-R¹¹ are D; and R¹²-R¹⁹ are H |
| 5 | Z = D; R¹-R⁹ are H; R¹⁰-R¹¹ are D; and R¹²-R¹⁹ are H |
| 6 | Z = D; R¹ = H; R² and R³ are D; R⁴-R⁷ are H; R⁸-R⁹ are D; R¹⁰-R¹⁴ are H; |
| | R¹⁵-R¹⁹ are D |

Another embodiment provides a compound in Table 12 wherein the compound has an enantiomeric excess of at least 60%, 70%, 75%, 80%, 85%, 90%, 95%, or 98%.

### Deuterium-Enriched Englitazone

Another collection of deuterium-enriched compounds is represented by Formula I-D having a stereochemical purity of at least 75% enantiomeric excess at the carbon bearing variable Z:

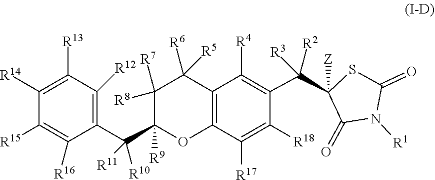

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are independently H or D.

In certain embodiments, R² and R³ are H. In certain embodiments, R¹⁰ and R¹¹ are H. In certain embodiments, R⁵, R⁶, R⁷, and R⁸ are H. In certain embodiments, R⁴, R¹⁷ and R¹⁸ are H. In certain embodiments, R¹², R¹³, R¹⁴, R¹⁵, and R¹⁶ are H. In certain other embodiments, R², R³, R¹⁰, and R¹¹ are H.

In certain embodiments, R², R³, R⁷, R⁸, R¹⁰, and R¹¹ are H. In certain embodiments, R², R³, R⁵, R⁶, R⁷, R⁸, and R⁹ are H. In certain embodiments, R⁵, R⁶, R⁷, R⁸, R¹², R¹³, R¹⁴, R¹⁵, and R¹⁶ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain embodiments, R⁹ is H. In certain other embodiments, R¹, R², R³, R⁴, R⁵, R⁶, R⁷, R⁸ and R⁹ are H.

The description above describes multiple embodiments relating to compounds of Formula I-D. The patent application specifically contemplates all combinations of the embodiments. For example, the invention contemplates a compound of Formula I-D wherein R², R³, R⁴, R⁵, R⁶, R⁷, R⁸, R⁹, R¹⁰, and R¹¹ are H.

Another collection of deuterium-enriched compounds is represented by Formula I-D1 having a stereochemical purity of at least 75% enantiomeric excess at the carbon atom bearing variable Z:

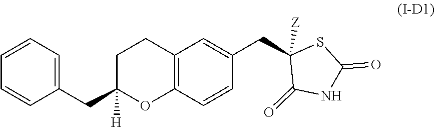

or a pharmaceutically acceptable salt thereof, wherein Z is H or D, provided that the abundance of deuterium in Z is at least 30%.

The compounds of Formula I-D and Formula I-D1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula I-D and Formula I-D1 can be further characterized according their stereochemical purity. In certain embodiments, the deuterium-enriched compound has a stereochemical purity of at least 80%, 85%, 90%, 95%, or 98% enantiomeric excess at the carbon atom bearing variable Z. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99% at the carbon atom bearing variable Z.

Still further such deuterium-enriched compounds are provided in Tables 13 and 14 below.

TABLE 13

| Compound | |
|---|---|
| No. | Structure |
| 1 | 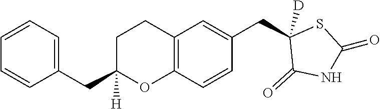 [SMILES: [2H][C@]1(Cc2ccc3c(c2)CC[C@H](Cc2ccccc2)O3)SC(=O)NC1=O] |
| | or a pharmaceutically acceptable salt |
| | thereof, each having a stereochemical purity of at least 60% enantiomeric |
| | excess at the carbon atom bearing D. |
| 2 | 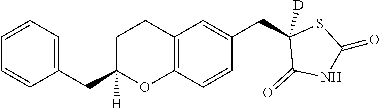 [SMILES: [2H][C@]1(Cc2ccc3c(c2)CC[C@H](Cc2ccccc2)O3)SC(=O)NC1=O] |
| | having a stereochemical purity of at |
| | least 60% enantiomeric excess at the carbon atom bearing D. |
| 3 | 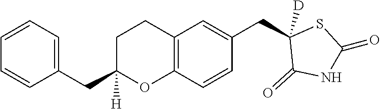 [SMILES: [2H][C@]1(Cc2ccc3c(c2)CC[C@H](Cc2ccccc2)O3)SC(=O)NC1=O] |
| | or a pharmaceutically acceptable salt |
| | thereof, each having a stereochemical purity of at least 75% enantiomeric |
| | excess at the carbon atom bearing D. |
| 4 | 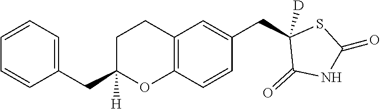 [SMILES: [2H][C@]1(Cc2ccc3c(c2)CC[C@H](Cc2ccccc2)O3)SC(=O)NC1=O] |
| | having a stereochemical purity of at |
| | least 75% enantiomeric excess at the carbon atom bearing D. |
| 5 | 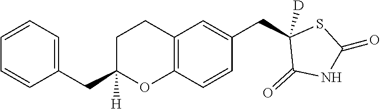 [SMILES: [2H][C@]1(Cc2ccc3c(c2)CC[C@H](Cc2ccccc2)O3)SC(=O)NC1=O] |
| | or a pharmaceutically acceptable salt |
| | thereof, each having a stereochemical purity of at least 90% enantiomeric |
| | excess at the carbon atom bearing D. |
| 6 | 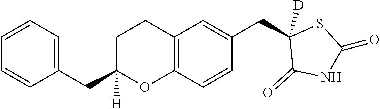 [SMILES: [2H][C@]1(Cc2ccc3c(c2)CC[C@H](Cc2ccccc2)O3)SC(=O)NC1=O] |
| | having a stereochemical purity of at |
| | least 90% enantiomeric excess at the carbon atom bearing D. |
| 7 | 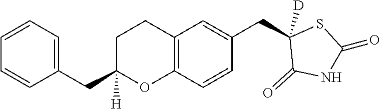 [SMILES: [2H][C@]1(Cc2ccc3c(c2)CC[C@H](Cc2ccccc2)O3)SC(=O)NC1=O] |
| | or a pharmaceutically acceptable salt |
| | thereof, each having a stereochemical purity of at least 95% enantiomeric |
| | excess at the carbon atom bearing D. |
| 8 | 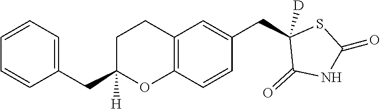 [SMILES: [2H][C@]1(Cc2ccc3c(c2)CC[C@H](Cc2ccccc2)O3)SC(=O)NC1=O] |
| | having a stereochemical purity of at |
| | least 95% enantiomeric excess at the carbon atom bearing D. |

TABLE 14

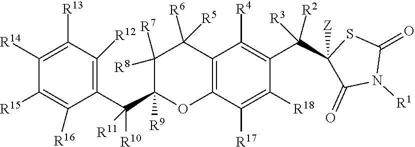

| Compound | |
|---|---|
| No. | Variable Definition |
| 1 | Z = D; R¹ = H; R²-R⁸ are H; R⁹ is D; and R¹⁰-R¹⁸ are H |
| 2 | Z = D; R¹ = H; R² and R³ are D; and R⁴-R¹⁸ are H |
| 3 | Z = D; R¹-R⁴ are H; R⁵-R⁸ are D; and R⁹-R¹⁸ are H |
| 4 | Z = D; R¹-R⁸ are H; R⁹-R¹¹ are D; and R¹²-R¹⁸ are H |
| 5 | Z = D; R¹-R⁹ are H; R¹⁰-R¹¹ are D; and R¹²-R¹⁸ are H |
| 6 | Z = D; R¹ = H; R² and R³ are D; R⁴-R⁸ are H; R⁹ is D; R¹⁰-R¹¹ are H; R¹²- |
| | R¹⁶ are D; and R¹⁷ and R¹⁸ are H |

Another embodiment provides a compound in Table 14 wherein the compound has an enantiomeric excess of at least 60%, 70%, 75%, 80%, 85%, 90%, 95%, or 98% at the carbon atom bearing variable Z.

Another collection of deuterium-enriched compounds is represented by Formula II-D having a stereochemical purity of at least 75% enantiomeric excess at the carbon bearing variable Z:

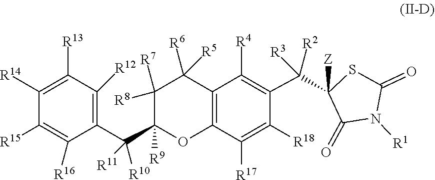

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are independently H or D.

In certain embodiments, R² and R³ are H. In certain embodiments, R¹⁰ and R¹¹ are H. In certain embodiments, R⁵, R⁶, R⁷, and R⁸ are H. In certain embodiments, R⁴, R¹⁷ and R¹⁸ are H. In certain embodiments, R¹², R¹³, R¹⁴, R¹⁵, and R¹⁶ are H. In certain other embodiments, R², R³, R¹⁰, and R¹¹ are H.

In certain embodiments, R², R³, R⁷, R⁸, R¹⁰, and R¹¹ are H. In certain embodiments, R², R³, R⁵, R⁶, R⁷, R⁸, and R⁹ are H. In certain embodiments, R⁵, R⁶, R⁷, R⁸, R¹², R¹³, R¹⁴, R¹⁵, and R¹⁶ are H.

In certain embodiments, R¹ is H. In certain embodiments, R² is H. In certain embodiments, R³ is H. In certain embodiments, R⁴ is H. In certain embodiments, R⁸ is H. In certain embodiments, R⁶ is H. In certain embodiments, R⁷ is H. In certain embodiments, R⁸ is H. In certain embodiments, R⁹ is H. In certain other embodiments, R¹, R², R³, R⁴, R⁵, R⁶, R⁷, R⁸ and R⁹ are H.

The description above describes multiple embodiments relating to compounds of Formula II-D. The patent application specifically contemplates all combinations of the embodiments. For example, the invention contemplates a compound of Formula II-D wherein R², R³, R⁴, R⁵, R⁶, R⁷, R⁸, R⁹, R¹⁰, and R¹¹ are H.

Another collection of deuterium-enriched compounds is represented by Formula II-D1 having a stereochemical purity of at least 75% enantiomeric excess at the carbon atom bearing variable Z:

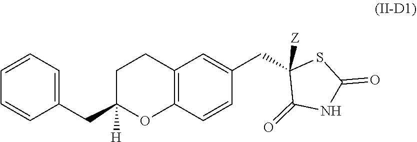

or a pharmaceutically acceptable salt thereof, wherein Z is H or D, provided that the abundance of deuterium in Z is at least 30%.

The compounds of Formula II-D and Formula II-D1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula II-D and Formula II-D1 can be further characterized according their enantiomeric purity. In certain embodiments, the deuterium-enriched compound has a stereochemical purity of at least 80%, 85%, 90%, 95%, or 98% enantiomeric excess at the carbon atom bearing variable Z. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99% at the carbon atom bearing variable Z.

Still further such deuterium-enriched compounds are provided in Tables 15 and 16 below.

TABLE 15

| Compound | |
|---|---|
| No. | Structure |
| 1 | 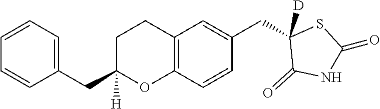 [SMILES: [2H][C@@]1(Cc2ccc3c(c2)CC[C@H](Cc2ccccc2)O3)SC(=O)NC1=O] |
| | or a pharmaceutically acceptable salt |
| | thereof, each having a stereochemical purity of at least 60% enantiomeric |
| | excess at the carbon atom bearing D. |
| 2 | 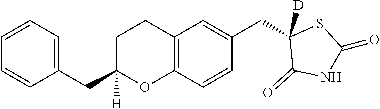 [SMILES: [2H][C@@]1(Cc2ccc3c(c2)CC[C@H](Cc2ccccc2)O3)SC(=O)NC1=O] |
| | having a stereochemical purity of at |
| | least 60% enantiomeric excess at the carbon atom bearing D. |
| 3 | 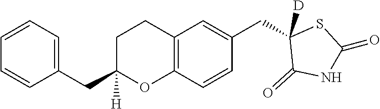 [SMILES: [2H][C@@]1(Cc2ccc3c(c2)CC[C@H](Cc2ccccc2)O3)SC(=O)NC1=O] |
| | or a pharmaceutically acceptable salt |
| | thereof, each having a stereochemical purity of at least 75% enantiomeric |
| | excess at the carbon atom bearing D. |
| 4 | 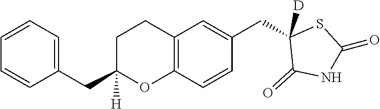 [SMILES: [2H][C@@]1(Cc2ccc3c(c2)CC[C@H](Cc2ccccc2)O3)SC(=O)NC1=O] |
| | having a stereochemical purity of at |
| | least 75% enantiomeric excess at the carbon atom bearing D. |
| 5 | 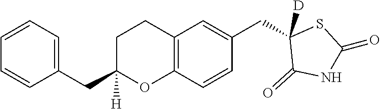 [SMILES: [2H][C@@]1(Cc2ccc3c(c2)CC[C@H](Cc2ccccc2)O3)SC(=O)NC1=O] |
| | or a pharmaceutically acceptable salt |
| | thereof, each having a stereochemical purity of at least 90% enantiomeric |
| | excess at the carbon atom bearing D. |
| 6 | 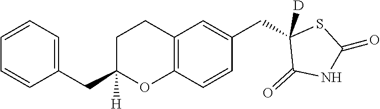 [SMILES: [2H][C@@]1(Cc2ccc3c(c2)CC[C@H](Cc2ccccc2)O3)SC(=O)NC1=O] |
| | having a stereochemical purity of at |
| | least 90% enantiomeric excess at the carbon atom bearing D. |
| 7 | 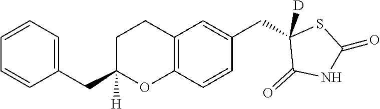 [SMILES: [2H][C@@]1(Cc2ccc3c(c2)CC[C@H](Cc2ccccc2)O3)SC(=O)NC1=O] |
| | or a pharmaceutically acceptable salt |
| | thereof, each having a stereochemical purity of at least 95% enantiomeric |
| | excess at the carbon atom bearing D. |
| 8 | 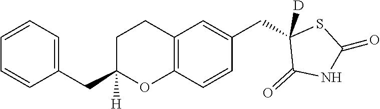 [SMILES: [2H][C@@]1(Cc2ccc3c(c2)CC[C@H](Cc2ccccc2)O3)SC(=O)NC1=O] |
| | having a stereochemical purity of at |
| | least 95% enantiomeric excess at the carbon atom bearing D. |

TABLE 16

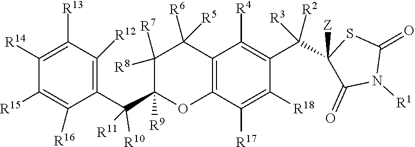

| Compound | |
|---|---|
| No. | Variable Definition |
| 1 | Z = D; R¹ = H; R²-R⁸ are H; R⁹ is D; and R¹⁰-R¹⁸ are H |
| 2 | Z = D; R¹ = H; R² and R³ are D; and R⁴-R¹⁸ are H |
| 3 | Z = D; R¹-R⁴ are H; R⁵-R⁸ are D; and R⁹-R¹⁸ are H |
| 4 | Z = D; R¹-R⁸ are H; R⁹-R¹¹ are D; and R¹²-R¹⁸ are H |
| 5 | Z = D; R¹-R⁹ are H; R¹⁰-R¹¹ are D; and R¹²-R¹⁸ are H |
| 6 | Z = D; R¹ = H; R² and R³ are D; R⁴-R⁸ are H; R⁹ is D; R¹⁰-R¹¹ are H; R¹²-R¹⁶ are |
| | D; and R¹⁷ and R¹⁸ are H |

Another embodiment provides a compound in Table 16 wherein the compound has an enantiomeric excess of at least 60%, 70%, 75%, 80%, 85%, 90%, 95%, or 98% at the carbon atom bearing variable Z.

### Deuterium-Enriched Lobeglitazone

Another collection of deuterium-enriched compounds is represented by Formula I-E having a stereochemical purity of at least 75% enantiomeric excess:

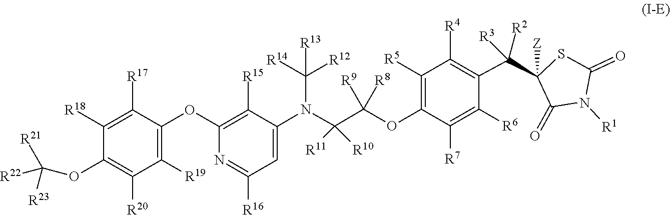

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹, R¹¹, R¹², R¹³, R¹⁴, R¹⁵, R¹⁶, R¹⁷, R¹⁸, R¹⁹, R²⁰, R²¹, R²², and R²³ are independently H or D.

In certain embodiments, R² and R³ are H. In certain embodiments, R⁸ and R⁹ are H. In certain embodiments, R⁴, R⁵, R⁶, and R⁷ are H. In certain embodiments, R⁸, R⁹, R¹⁰, and R¹¹ are H. In certain embodiments, R¹⁵, R¹⁶, R¹⁷, R¹⁸, R¹⁹, and R²⁰ are H. In certain other embodiments, R², R³, R⁸, and R⁹ are H.

In certain embodiments, R², R³, R⁸, R⁹, R¹⁰, and R¹¹ are H. In certain embodiments, R⁴, R⁵, R⁶, R⁷, R⁸, and R⁹ are H. In certain embodiments, R⁴, R⁵, R⁶, R⁷, R¹², R¹³, R¹⁴, R¹⁵, R¹⁶, R²¹, R²², and R²³ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, R⁸ and R⁹ are H.

The description above describes multiple embodiments relating to compounds of Formula I-E. The patent application specifically contemplates all combinations of the embodiments. For example, the invention contemplates a compound of Formula I-E wherein R², R³, R⁴, R⁵, R⁶, R⁷, R⁸, R⁹, R¹⁰, and R¹¹ are H.

Another collection of deuterium-enriched compounds is represented by Formula I-E1 having a stereochemical purity of at least 75% enantiomeric excess:

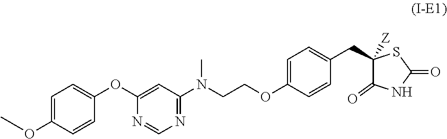

or a pharmaceutically acceptable salt thereof, wherein Z is H or D, provided that the abundance of deuterium in Z is at least 30%.

The compounds of Formula I-E and Formula I-E1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula I-E and Formula I-E1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 17 and 18 below.

TABLE 17

| Compound | |
|---|---|
| No. | Structure |
| 1 | 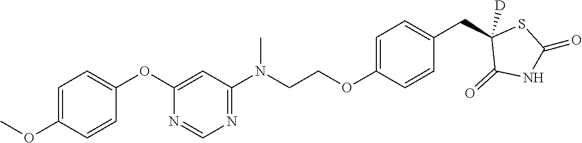 [SMILES: [2H][C@]1(Cc2ccc(OCCN(C)c3cc(Oc4ccc(OC)cc4)ncn3)cc2)SC(=O)NC1=O] |
| | or a |
| | pharmaceutically acceptable salt thereof, each having a stereochemical |
| | purity of at least 60% enantiomeric excess. |
| 2 | 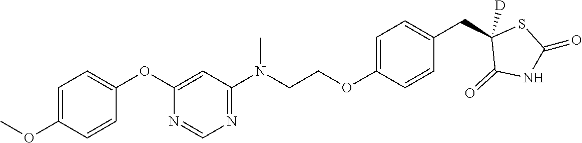 [SMILES: [2H][C@]1(Cc2ccc(OCCN(C)c3cc(Oc4ccc(OC)cc4)ncn3)cc2)SC(=O)NC1=O] |
| | having a |
| | stereochemical purity of at least 60% enantiomeric excess. |
| 3 | 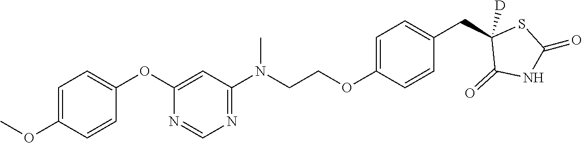 [SMILES: [2H][C@]1(Cc2ccc(OCCN(C)c3cc(Oc4ccc(OC)cc4)ncn3)cc2)SC(=O)NC1=O] |
| | or a |
| | pharmaceutically acceptable salt thereof, each having a stereochemical |
| | purity of at least 75% enantiomeric excess. |
| 4 | 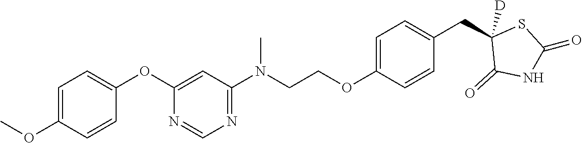 [SMILES: [2H][C@]1(Cc2ccc(OCCN(C)c3cc(Oc4ccc(OC)cc4)ncn3)cc2)SC(=O)NC1=O] |
| | having a |
| | stereochemical purity of at least 75% enantiomeric excess. |
| 5 | 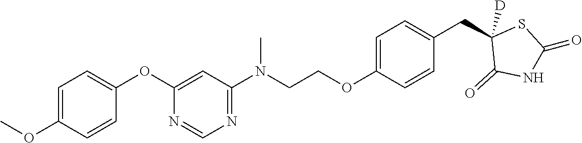 [SMILES: [2H][C@]1(Cc2ccc(OCCN(C)c3cc(Oc4ccc(OC)cc4)ncn3)cc2)SC(=O)NC1=O] |
| | or a |
| | pharmaceutically acceptable salt thereof, each having a stereochemical |
| | purity of at least 90% enantiomeric excess. |
| 6 | 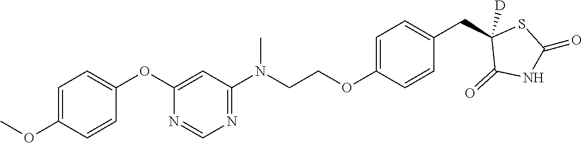 [SMILES: [2H][C@]1(Cc2ccc(OCCN(C)c3cc(Oc4ccc(OC)cc4)ncn3)cc2)SC(=O)NC1=O] |
| | having a |
| | stereochemical purity of at least 90% enantiomeric excess. |
| 7 | 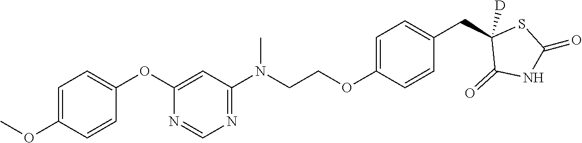 [SMILES: [2H][C@]1(Cc2ccc(OCCN(C)c3cc(Oc4ccc(OC)cc4)ncn3)cc2)SC(=O)NC1=O] |
| | or a |
| | pharmaceutically acceptable salt thereof, each having a stereochemical |
| | purity of at least 95% enantiomeric excess. |
| 8 | 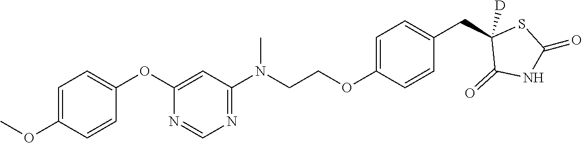 [SMILES: [2H][C@]1(Cc2ccc(OCCN(C)c3cc(Oc4ccc(OC)cc4)ncn3)cc2)SC(=O)NC1=O] |
| | having a |
| | stereochemical purity of at least 95% enantiomeric excess. |

TABLE 18

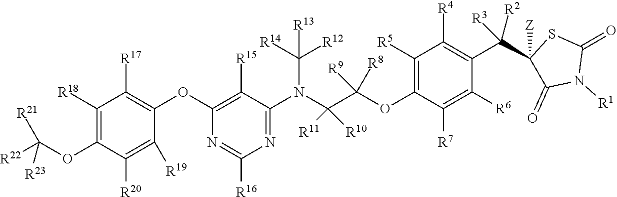

| Compound | |
|---|---|
| No. | Variable Definition |
| 1 | Z = D; R¹ = H; R²-R⁹ are D; and R¹⁰-R²³ are H |
| 2 | Z = D; R¹ = H; R² and R³ are D; and R⁴-R²³ are H |
| 3 | Z = D; R¹-R³ are H; R⁴-R⁷ are D; and R⁸-R²³ are H |
| 4 | Z = D; R¹-R⁷ are H; R⁸-R¹¹ are D; and R¹²-R²³ are H |
| 5 | Z = D; R¹-R⁹ are H; R¹⁰-R¹¹ are D; and R¹²-R²³ are H |
| 6 | Z = D; R¹ = H; R² and R³ are D; R⁴-R⁷ are H; R⁸-R⁹ are D; R¹⁰-R¹⁴ are H; and R¹⁵-R²³ are D |

Another embodiment provides a compound in Table 18 wherein the compound has an enantiomeric excess of at least 60%, 70%, 75%, 80%, 85%, 90%, 95%, or 98%.

Another collection of deuterium-enriched compounds is represented by Formula II-E having a stereochemical purity of at least 75% enantiomeric excess:

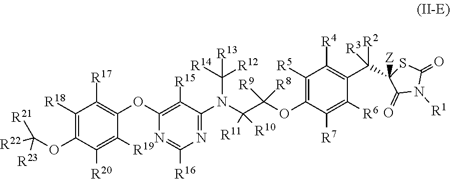

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², and R²³ are independently H or D.

In certain embodiments, R² and R³ are H. In certain embodiments, R⁸ and R⁹ are H. In certain embodiments, R⁴, R⁵, R⁶, and R⁷ are H. In certain embodiments, R⁸, R⁹, R¹⁰, and R¹¹ are H. In certain embodiments, R¹⁵, R¹⁶, R¹⁷, R¹⁸, R¹⁹, and R²⁰ are H. In certain other embodiments, R², R³, R⁸, and R⁹ are H.

In certain embodiments, R², R³, R⁸, R⁹, R¹⁰, and R¹¹ are H. In certain embodiments, R⁴, R⁵, R⁶, R⁷, R⁸, and R⁹ are H. In certain embodiments, R⁴, R⁵, R⁶, R⁷, R¹², R¹³, R¹⁴, R⁵, R¹⁶, R²¹, R²², and R²³ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, R⁸ and R⁹ are H.

The description above describes multiple embodiments relating to compounds of Formula II-E. The patent application specifically contemplates all combinations of the embodiments. For example, the invention contemplates a compound of Formula II-E wherein R², R³, R⁴, R⁵, R⁶, R⁷, R⁸, R⁹, R¹⁰, and R¹¹ are H.

Another collection of deuterium-enriched compounds is represented by Formula II-E1 having a stereochemical purity of at least 75% enantiomeric excess:

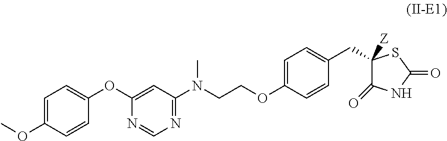

or a pharmaceutically acceptable salt thereof, wherein Z is H or D, provided that the abundance of deuterium in Z is at least 30%.

The compounds of Formula II-E and Formula II-E1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula II-E and Formula II-E1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 19 and 20 below.

TABLE 19

| Compound | |
|---|---|
| No. | Structure |
| 1 | 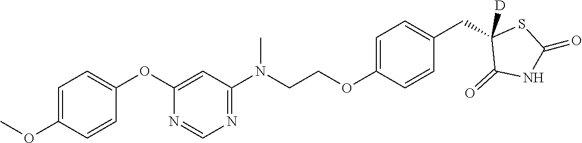 [SMILES: [2H][C@@]1(Cc2ccc(OCCN(C)c3cc(Oc4ccc(OC)cc4)ncn3)cc2)SC(=O)NC1=O] |
| | or a |
| | pharmaceutically acceptable salt thereof, each having a stereochemical |
| | purity of at least 60% enantiomeric excess. |
| 2 | 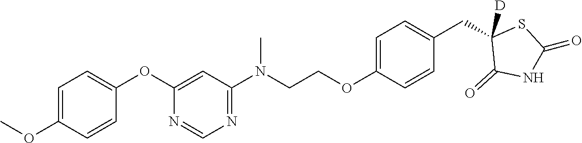 [SMILES: [2H][C@@]1(Cc2ccc(OCCN(C)c3cc(Oc4ccc(OC)cc4)ncn3)cc2)SC(=O)NC1=O] |
| | having a |
| | stereochemical purity of at least 60% enantiomeric excess. |
| 3 | 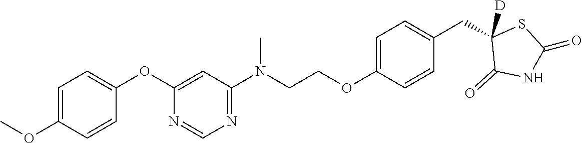 [SMILES: [2H][C@@]1(Cc2ccc(OCCN(C)c3cc(Oc4ccc(OC)cc4)ncn3)cc2)SC(=O)NC1=O] |
| | or a |
| | pharmaceutically acceptable salt thereof, each having a stereochemical |
| | purity of at least 75% enantiomeric excess. |
| 4 | 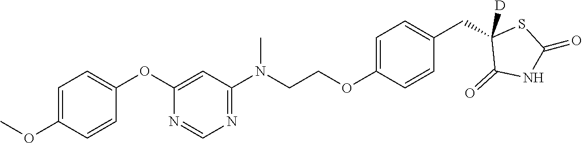 [SMILES: [2H][C@@]1(Cc2ccc(OCCN(C)c3cc(Oc4ccc(OC)cc4)ncn3)cc2)SC(=O)NC1=O] |
| | having a |
| | stereochemical purity of at least 75% enantiomeric excess. |
| 5 | 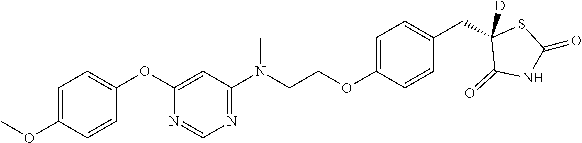 [SMILES: [2H][C@@]1(Cc2ccc(OCCN(C)c3cc(Oc4ccc(OC)cc4)ncn3)cc2)SC(=O)NC1=O] |
| | or a |
| | pharmaceutically acceptable salt thereof, each having a stereochemical |
| | purity of at least 90% enantiomeric excess. |
| 6 | 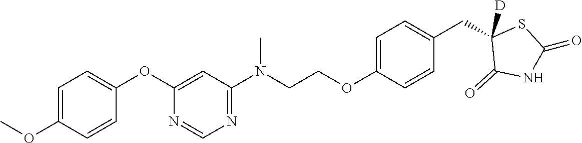 [SMILES: [2H][C@@]1(Cc2ccc(OCCN(C)c3cc(Oc4ccc(OC)cc4)ncn3)cc2)SC(=O)NC1=O] |
| | having a |
| | stereochemical purity of at least 90% enantiomeric excess. |
| 7 | 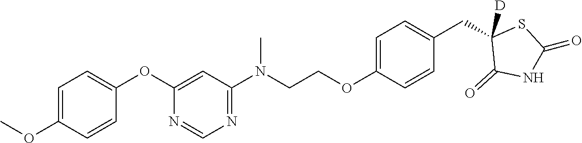 [SMILES: [2H][C@@]1(Cc2ccc(OCCN(C)c3cc(Oc4ccc(OC)cc4)ncn3)cc2)SC(=O)NC1=O] |
| | or a |
| | pharmaceutically acceptable salt thereof, each having a stereochemical |
| | purity of at least 95% enantiomeric excess. |
| 8 | 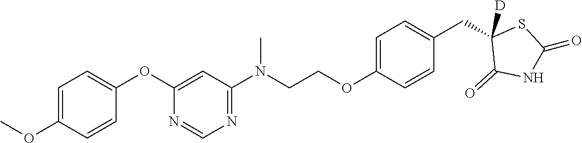 [SMILES: [2H][C@@]1(Cc2ccc(OCCN(C)c3cc(Oc4ccc(OC)cc4)ncn3)cc2)SC(=O)NC1=O] |
| | having a |
| | stereochemical purity of at least 95% enantiomeric excess. |

TABLE 20

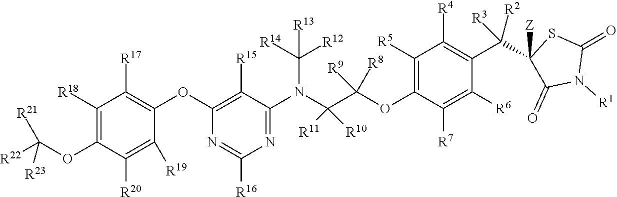

| Compound | |
|---|---|
| No. | Variable Definition |
| 1 | Z = D; R¹ = H; R²-R⁹ are D; and R¹⁰-R²³ are H |
| 2 | Z = D; R¹ = H; R² and R³ are D; and R⁴-R²³ are H |
| 3 | Z = D; R¹-R³ are H; R⁴-R⁷ are D; and R⁸-R²³ are H |
| 4 | Z = D; R¹-R⁷ are H; R⁸-R¹¹ are D; and R¹²-R²³ are H |
| 5 | Z = D; R¹-R⁹ are H; R¹⁰-R¹¹ are D; and R¹²-R²³ are H |
| 6 | Z = D; R¹ = H; R² and R³ are D; R⁴-R⁷ are H; R⁸-R⁹ are D; R¹⁰-R¹⁴ are H; |
| | and R¹⁵-R²³ are D |

Another embodiment provides a compound in Table 20 wherein the compound has an enantiomeric excess of at least 60%, 70%, 75%, 80%, 85%, 90%, 95%, or 98%.

### Deuterium-Enriched MK-0767

Another collection of deuterium-enriched compounds is represented by Formula I-F having a stereochemical purity of at least 75% enantiomeric excess:

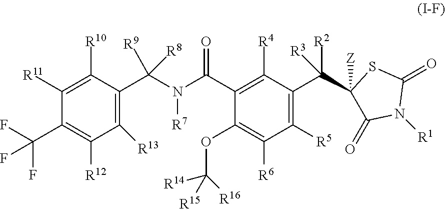

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, and R¹⁶ are independently H or D.

In certain embodiments, R² and R³ are H. In certain embodiments, R⁸ and R⁹ are H. In certain embodiments, R⁴, R⁵, R⁶, and R⁷ are H. In certain embodiments, R⁸, R⁹, R¹⁰, R¹¹, R¹², and R¹³ are H. In certain embodiments, R¹⁴, R^{L}, and R¹⁶ are H. In certain other embodiments, R², R³, R⁸, and R⁹ are H.

In certain embodiments, R², R³, R⁸, R⁹, R¹⁰, R¹¹, R¹², and R¹³ are H. In certain embodiments, R⁴, R⁵, R⁶, R⁷, R⁸, and R⁹ are H. In certain embodiments, R⁴, R⁵, R⁶, R⁷, R¹⁰, R¹¹, R¹², R¹³, R¹⁴, R¹⁵, and R¹⁶ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, R⁸ and R⁹ are H.

The description above describes multiple embodiments relating to compounds of Formula I-F. The patent application specifically contemplates all combinations of the embodiments. For example, the invention contemplates a compound of Formula I-F wherein R², R³, R⁴, R⁵, R⁶, R⁷, R⁸, R⁹, R¹⁰, R¹¹, R, and R¹³ are H.

Another collection of deuterium-enriched compounds is represented by Formula I-F1 having a stereochemical purity of at least 75% enantiomeric excess:

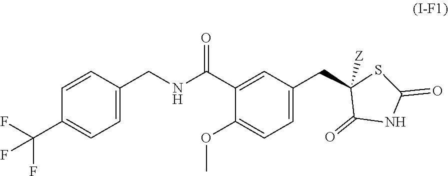

or a pharmaceutically acceptable salt thereof, wherein Z is H or D, provided that the abundance of deuterium in Z is at least 30%.

The compounds of Formula I-F and Formula I-F1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula I-F and Formula I-F1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 21 and 22 below.

TABLE 21

| Compound | |
|---|---|
| No. | Structure |
| 1 | 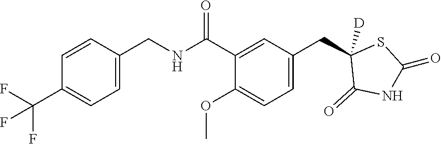 [SMILES: [2H][C@]1(Cc2ccc(OC)c(C(=O)NCc3ccc(C(F)(F)F)cc3)c2)SC(=O)NC1=O] |
| | or a pharmaceutically |
| | acceptable salt thereof, each having a stereochemical purity of at least 60% |
| | enantiomeric excess. |
| 2 | 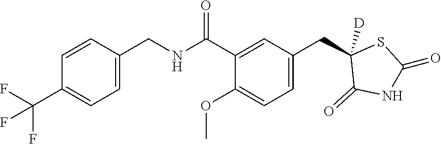 [SMILES: [2H][C@]1(Cc2ccc(OC)c(C(=O)NCc3ccc(C(F)(F)F)cc3)c2)SC(=O)NC1=O] |
| | having a stereochemical purity |
| | of at least 60% enantiomeric excess. |
| 3 | 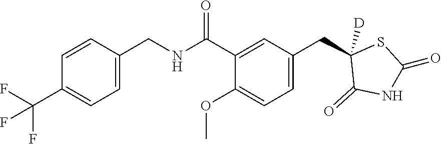 [SMILES: [2H][C@]1(Cc2ccc(OC)c(C(=O)NCc3ccc(C(F)(F)F)cc3)c2)SC(=O)NC1=O] |
| | or a pharmaceutically |
| | acceptable salt thereof, each having a stereochemical purity of at least 75% |
| | enantiomeric excess. |
| 4 | 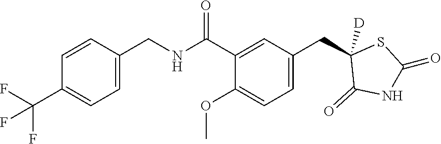 [SMILES: [2H][C@]1(Cc2ccc(OC)c(C(=O)NCc3ccc(C(F)(F)F)cc3)c2)SC(=O)NC1=O] |
| | having a stereochemical purity |
| | of at least 75% enantiomeric excess. |
| 5 | 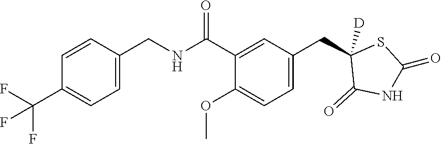 [SMILES: [2H][C@]1(Cc2ccc(OC)c(C(=O)NCc3ccc(C(F)(F)F)cc3)c2)SC(=O)NC1=O] |
| | or a pharmaceutically |
| | acceptable salt thereof, each having a stereochemical purity of at least 90% |
| | enantiomeric excess. |
| 6 | 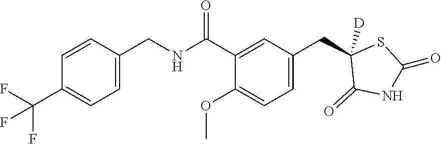 [SMILES: [2H][C@]1(Cc2ccc(OC)c(C(=O)NCc3ccc(C(F)(F)F)cc3)c2)SC(=O)NC1=O] |
| | having a stereochemical purity |
| | of at least 90% enantiomeric excess. |
| 7 | 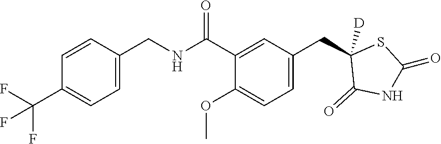 [SMILES: [2H][C@]1(Cc2ccc(OC)c(C(=O)NCc3ccc(C(F)(F)F)cc3)c2)SC(=O)NC1=O] |
| | or a pharmaceutically |
| | acceptable salt thereof, each having a stereochemical purity of at least 95% |
| | enantiomeric excess. |
| 8 | 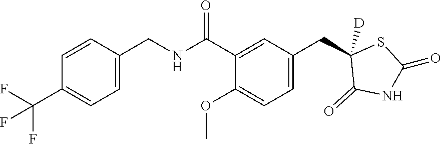 [SMILES: [2H][C@]1(Cc2ccc(OC)c(C(=O)NCc3ccc(C(F)(F)F)cc3)c2)SC(=O)NC1=O] |
| | having a stereochemical purity |
| | of at least 95% enantiomeric excess. |

TABLE 22

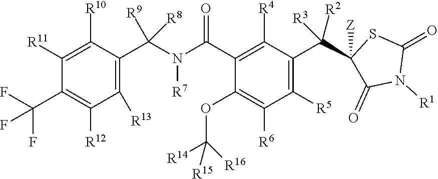

| Compound | |
|---|---|
| No. | Variable Definition |
| 1 | Z = D; R¹ = H; R²-R⁶ are D; R⁷ = H; R⁸ and R⁹ are D; and R¹⁰-R¹⁶ are H |
| 2 | Z = D; R¹ = H; R² and R³ are D; and R⁴-R¹⁶ are H |
| 3 | Z = D; R¹-R³ are H; R⁴-R⁶ are D; and R⁷-R¹⁶ are H |
| 4 | Z = D; R¹-R⁷ are H; R⁸-R¹³ are D; and R¹⁴-R¹⁶ are H |
| 5 | Z = D; R¹-R⁷ are H; R⁸ and R⁹ are D; and R¹⁰-R¹⁶ are H |
| 6 | Z = D; R¹ = H; R² and R³ are D; R⁴-R⁷ are H; R⁸-R⁹ are D; R¹⁰-R¹³ are H; |
| | and R¹⁴-R¹⁶ are D |

Another embodiment provides a compound in Table 22 wherein the compound has an enantiomeric excess of at least 60%, 70%, 75%, 80%, 85%, 90%, 95%, or 98%.

Another collection of deuterium-enriched compounds is represented by Formula II-F having a stereochemical purity of at least 75% enantiomeric excess:

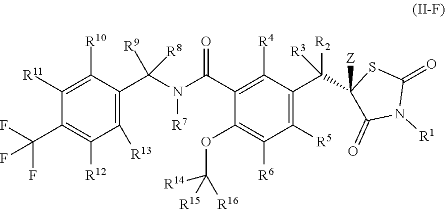

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, and R¹⁶ are independently H or D.

In certain embodiments, R² and R³ are H. In certain embodiments, R⁸ and R⁹ are H. In certain embodiments, R⁴, R⁵, R⁶, and R⁷ are H. In certain embodiments, R⁸, R⁹, R¹⁰, R¹¹ R¹², and R¹³ are H. In certain embodiments, R¹⁴, R⁵, and R¹⁶ are H. In certain other embodiments, R², R³, R⁸, and R⁹ are H.

In certain embodiments, R², R³, R⁸, R⁹, R¹⁰, R¹¹, R¹², and R¹³ are H. In certain embodiments, R⁴, R⁵, R⁶, R⁷, R⁸, and R⁹ are H. In certain embodiments, R⁴, R⁵, R⁶, R⁷, R¹⁰, R¹¹, R¹², R¹³, R¹⁴, R¹⁵, and R¹⁶ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, R⁸ and R⁹ are H.

The description above describes multiple embodiments relating to compounds of Formula II-F. The patent application specifically contemplates all combinations of the embodiments. For example, the invention contemplates a compound of Formula II-F wherein R², R³, R⁴, R⁵, R⁶, R⁷, R⁸, R⁹, R¹⁰, R¹¹, R¹², and R¹³ are H.

Another collection of deuterium-enriched compounds is represented by Formula II-F1 having a stereochemical purity of at least 75% enantiomeric excess:

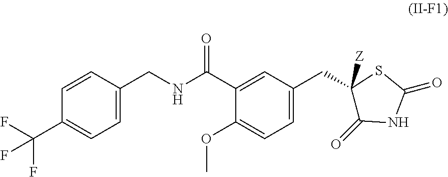

or a pharmaceutically acceptable salt thereof, wherein Z is H or D, provided that the abundance of deuterium in Z is at least 30%.

The compounds of Formula II-F and Formula II-F1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula II-F and Formula II-F1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 23 and 24 below.

TABLE 23

| Compound | |
|---|---|
| No. | Structure |
| 1 | 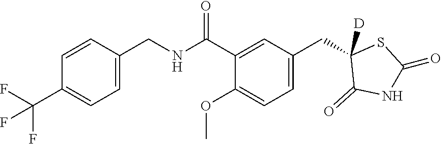 [SMILES: [2H][C@@]1(Cc2ccc(OC)c(C(=O)NCc3ccc(C(F)(F)F)cc3)c2)SC(=O)NC1=O] |
| | or a pharmaceutically |
| | acceptable salt thereof, each having a stereochemical purity of at least 60% |
| | enantiomeric excess. |
| 2 | 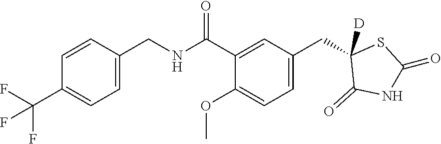 [SMILES: [2H][C@@]1(Cc2ccc(OC)c(C(=O)NCc3ccc(C(F)(F)F)cc3)c2)SC(=O)NC1=O] |
| | having a stereochemical purity |
| | of at least 60% enantiomeric excess. |
| 3 | 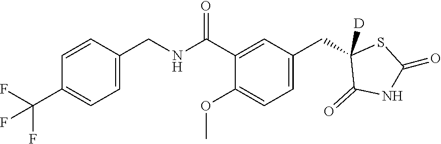 [SMILES: [2H][C@@]1(Cc2ccc(OC)c(C(=O)NCc3ccc(C(F)(F)F)cc3)c2)SC(=O)NC1=O] |
| | or a pharmaceutically |
| | acceptable salt thereof, each having a stereochemical purity of at least 75% |
| | enantiomeric excess. |
| 4 | 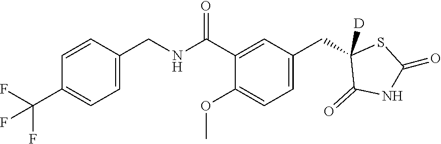 [SMILES: [2H][C@@]1(Cc2ccc(OC)c(C(=O)NCc3ccc(C(F)(F)F)cc3)c2)SC(=O)NC1=O] |
| | having a stereochemical purity |
| | of at least 75% enantiomeric excess. |
| 5 | 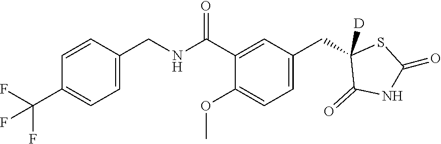 [SMILES: [2H][C@@]1(Cc2ccc(OC)c(C(=O)NCc3ccc(C(F)(F)F)cc3)c2)SC(=O)NC1=O] |
| | or a pharmaceutically |
| | acceptable salt thereof, each having a stereochemical purity of at least 90% |
| | enantiomeric excess. |
| 6 | 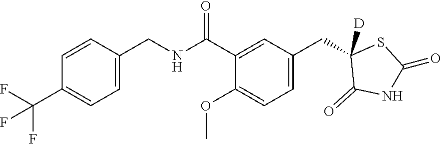 [SMILES: [2H][C@@]1(Cc2ccc(OC)c(C(=O)NCc3ccc(C(F)(F)F)cc3)c2)SC(=O)NC1=O] |
| | having a stereochemical purity |
| | of at least 90% enantiomeric excess. |
| 7 | 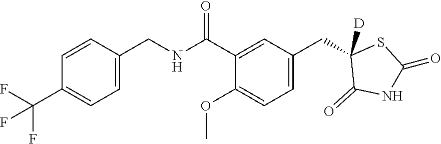 [SMILES: [2H][C@@]1(Cc2ccc(OC)c(C(=O)NCc3ccc(C(F)(F)F)cc3)c2)SC(=O)NC1=O] |
| | or a pharmaceutically |
| | acceptable salt thereof, each having a stereochemical purity of at least 95% |
| | enantiomeric excess. |
| 8 | 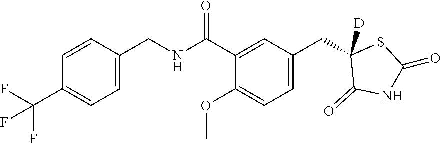 [SMILES: [2H][C@@]1(Cc2ccc(OC)c(C(=O)NCc3ccc(C(F)(F)F)cc3)c2)SC(=O)NC1=O] |
| | having a stereochemical purity |
| | of at least 95% enantiomeric excess. |

TABLE 24

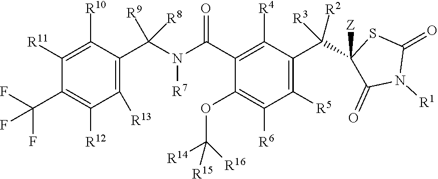

| Compound | |
|---|---|
| No. | Variable Definition |
| 1 | Z = D; R¹ = H; R²-R⁶ are D; R⁷= H; R⁸ and R⁹ are D; and R¹⁰-R¹⁶ are H |
| 2 | Z = D; R¹ = H; R² and R³ are D; and R⁴-R¹⁶ are H |
| 3 | Z = D; R¹-R³ are H; R⁴-R⁶ are D; and R⁷-R¹⁶ are H |
| 4 | Z = D; R¹-R⁷ are H; R⁸-R¹³ are D; and R¹⁴-R¹⁶ are H |
| 5 | Z = D; R¹-R⁷ are H; R⁸ and R⁹ are D; and R¹⁰-R¹⁶ are H |
| 6 | Z = D; R¹ = H; R² and R³ are D; R⁴-R⁷ are H; R⁸-R⁹ are D; R¹⁰-R¹³ are H; |
| | and R¹⁴-R¹⁶ are D |

Another embodiment provides a compound in Table 24 wherein the compound has an enantiomeric excess of at least 60%, 70%, 75%, 80%, 85%, 90%, 95%, or 98%.

Another collection of deuterium-enriched compounds is represented by Formula III:

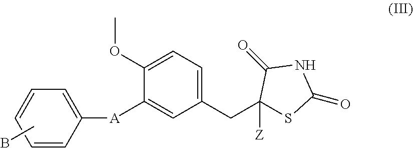

or a pharmaceutically acceptable salt thereof, wherein:

Z is H or D, provided that the abundance of deuterium in Z is at least 30%;

A is *—CH₂N(H)C(O)—, *—CH₂CONH—, *—NHCONH—, *—CH₂CH₂CO—, or *—NHCOCH₂—, wherein * is a bond to the phenyl group bearing substituent B;

B is C₁-C₄ alkyl, C₁-C₄ alkoxy, halogen, trifluoromethyl, trifluoro-methoxy, a phenyl group which is unsubstituted or may have substituents, a phenoxy group which is unsubstituted or may have substituents, or a benzyloxy group which is unsubstituted or may have substituents; and

any hydrogen atom may be optionally replaced with D.

In certain embodiments, the compound of Formula III is the S-enantiomer having a stereochemical purity of at least 75% enantiomeric excess. In certain embodiments, the compound of Formula III is the R-enantiomer having a stereochemical purity of at least 75% enantiomeric excess.

### Deuterium-Enriched Netoglitazone

Another collection of deuterium-enriched compounds is represented by Formula I-G having a stereochemical purity of at least 75% enantiomeric excess:

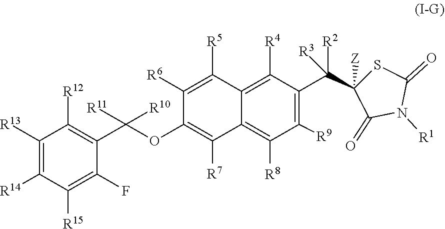

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, and R¹⁵ are independently H or D.

In certain embodiments, R² and R³ are H. In certain embodiments, R¹⁰ and R¹¹ are H. In certain embodiments, R⁴, R⁵, R⁶, R⁷, R⁸, and R⁹ are H. In certain embodiments, R¹², R¹³, R¹⁴, and R¹⁵ are H. In certain other embodiments, R², R³, R¹⁰, and R¹¹ are H.

In certain embodiments, R², R³, R¹⁰, R¹¹, R¹², R¹³, R¹⁴, and R¹⁵ are H. In certain embodiments, R⁴, R⁵, R⁶, R⁷, R⁸, R⁹, R¹⁰, and R¹¹ are H. In certain embodiments, R⁴, R⁵, R⁶, R⁷, R⁸, R⁹, R¹², R¹, R¹⁴, and R¹⁵ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain other embodiments, R¹, R², R³, R⁴, R⁵, R⁶, R⁷, R⁸ and R⁹ are H.

The description above describes multiple embodiments relating to compounds of Formula I-G. The patent application specifically contemplates all combinations of the embodiments. For example, the invention contemplates a compound of Formula I-G wherein R², R³, R⁴, R⁵, R⁶, R⁷, R⁸, R⁹, R¹⁰, and R¹¹ are H.

Another collection of deuterium-enriched compounds is represented by Formula I-G1 having a stereochemical purity of at least 75% enantiomeric excess:

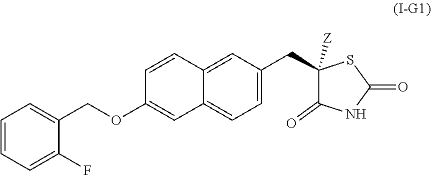

or a pharmaceutically acceptable salt thereof, wherein Z is H or D, provided that the abundance of deuterium in Z is at least 30%.

The compounds of Formula I-G and Formula I-G1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula I-G and Formula I-G1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 25 and 26 below.

TABLE 25

| Compound | |
|---|---|
| No. | Structure |
| 1 | 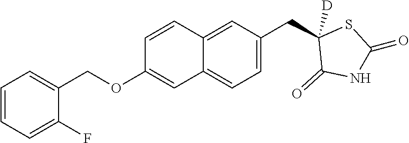 [SMILES: [2H][C@]1(Cc2ccc3cc(OCc4ccccc4F)ccc3c2)SC(=O)NC1=O] |
| | or a pharmaceutically acceptable |
| | salt thereof, each having a stereochemical purity of at least 60% |
| | enantiomeric excess. |
| 2 | 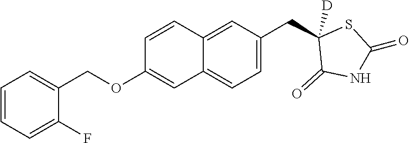 [SMILES: [2H][C@]1(Cc2ccc3cc(OCc4ccccc4F)ccc3c2)SC(=O)NC1=O] |
| | having a stereochemical purity of at |
| | least 60% enantiomeric excess. |
| 3 | 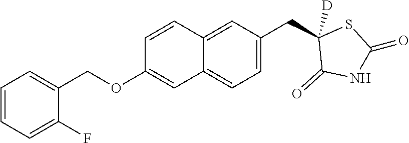 [SMILES: [2H][C@]1(Cc2ccc3cc(OCc4ccccc4F)ccc3c2)SC(=O)NC1=O] |
| | or a pharmaceutically acceptable |
| | salt thereof, each having a stereochemical purity of at least 75% |
| | enantiomeric excess. |
| 4 | 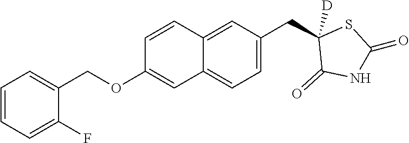 [SMILES: [2H][C@]1(Cc2ccc3cc(OCc4ccccc4F)ccc3c2)SC(=O)NC1=O] |
| | having a stereochemical purity of at |
| | least 75% enantiomeric excess. |
| 5 | 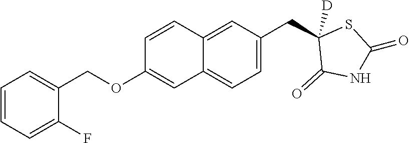 [SMILES: [2H][C@]1(Cc2ccc3cc(OCc4ccccc4F)ccc3c2)SC(=O)NC1=O] |
| | or a pharmaceutically acceptable |
| | salt thereof, each having a stereochemical purity of at least 90% |
| | enantiomeric excess. |
| 6 | 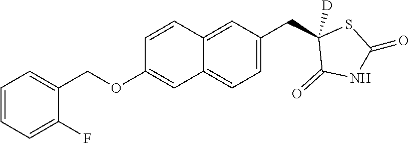 [SMILES: [2H][C@]1(Cc2ccc3cc(OCc4ccccc4F)ccc3c2)SC(=O)NC1=O] |
| | having a stereochemical purity of at |
| | least 90% enantiomeric excess. |
| 7 | 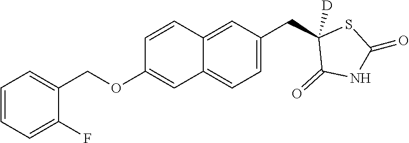 [SMILES: [2H][C@]1(Cc2ccc3cc(OCc4ccccc4F)ccc3c2)SC(=O)NC1=O] |
| | or a pharmaceutically acceptable |
| | salt thereof, each having a stereochemical purity of at least 95% |
| | enantiomeric excess. |
| 8 | 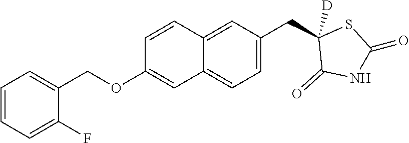 [SMILES: [2H][C@]1(Cc2ccc3cc(OCc4ccccc4F)ccc3c2)SC(=O)NC1=O] |
| | having a stereochemical purity of at |
| | least 95% enantiomeric excess. |

TABLE 26

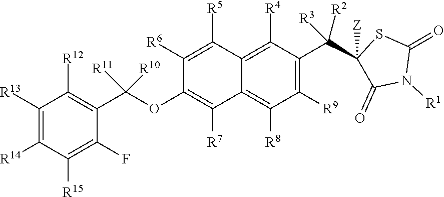

| Compound | |
|---|---|
| No. | Variable Definition |
| 1 | Z = D; R¹ = H; R²-R⁹ are D; and R¹⁰-R¹⁵ are H |
| 2 | Z = D; R¹ = H; R² and R³ are D; and R⁴-R¹⁵ are H |
| 3 | Z = D; R¹-R³ are H; R⁴-R⁹ are D; and R¹⁰-R¹⁵ are H |
| 4 | Z = D; R¹-R¹¹ are H; and R¹²-R¹⁵ are D |
| 5 | Z = D; R¹-R⁹ are H; R¹⁰ and R¹¹ are D; and R¹²-R¹⁵ are H |
| 6 | Z = D; R¹ = H; R² and R³ are D; R⁴-R⁹ are H; and R¹⁰-R¹⁵ are D |

Another embodiment provides a compound in Table 26 wherein the compound has an enantiomeric excess of at least 60%, 70%, 75%, 80%, 85%, 90%, 95%, or 98%.

Another collection of deuterium-enriched compounds is represented by Formula II-G having a stereochemical purity of at least 75% enantiomeric excess:

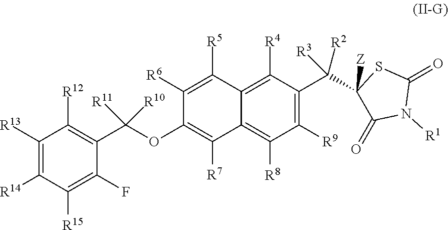

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, and R¹⁵ are independently H or D.

In certain embodiments, R² and R³ are H. In certain embodiments, R¹⁰ and R¹¹ are H. In certain embodiments, R⁴, R⁵, R⁶, R⁷, R⁸, and R⁹ are H. In certain embodiments, R¹², R¹³, R¹⁴, and R¹⁵ are H. In certain other embodiments, R², R³, R¹⁰, and R¹¹ are H.

In certain embodiments, R², R³, R¹⁰, R¹¹, R¹², R¹³, R¹⁴, and R¹⁵ are H. In certain embodiments, R⁴, R⁵, R⁶, R⁷, R⁸, R⁹, R¹⁰, and R¹¹ are H. In certain embodiments, R⁴, R⁵, R⁶, R⁷, R⁸, R⁹, R¹², R¹³, R¹⁴, and R¹⁵ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain other embodiments, R¹, R², R³, R⁴, R⁵, R⁶, R⁷, R⁸ and R⁹ are H.

The description above describes multiple embodiments relating to compounds of Formula II-G. The patent application specifically contemplates all combinations of the embodiments. For example, the invention contemplates a compound of Formula II-G wherein R², R³, R⁴, R⁵, R⁶, R⁷, R⁸, R⁹, R¹⁰, and R¹¹ are H.

Another collection of deuterium-enriched compounds is represented by Formula II-G1 having a stereochemical purity of at least 75% enantiomeric excess:

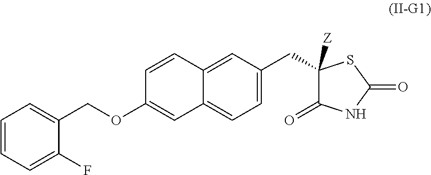

or a pharmaceutically acceptable salt thereof, wherein Z is H or D, provided that the abundance of deuterium in Z is at least 30%.

The compounds of Formula II-G and Formula II-G1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula II-G and Formula II-G1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 27 and 28 below.

TABLE 27

| Compound | |
|---|---|
| No. | Structure |
| 1 | 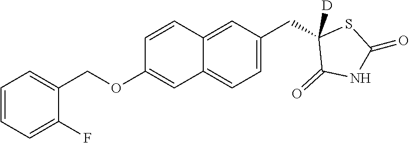 [SMILES: [2H][C@@]1(Cc2ccc3cc(OCc4ccccc4F)ccc3c2)SC(=O)NC1=O] |
| | or a pharmaceutically acceptable |
| | salt thereof, each having a stereochemical purity of at least 60% |
| | enantiomeric excess. |
| 2 | 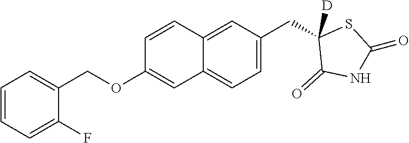 [SMILES: [2H][C@@]1(Cc2ccc3cc(OCc4ccccc4F)ccc3c2)SC(=O)NC1=O] |
| | having a stereochemical purity of at |
| | least 60% enantiomeric excess. |
| 3 | 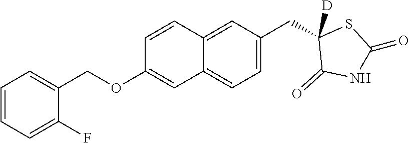 [SMILES: [2H][C@@]1(Cc2ccc3cc(OCc4ccccc4F)ccc3c2)SC(=O)NC1=O] |
| | or a pharmaceutically acceptable |
| | salt thereof, each having a stereochemical purity of at least 75% |
| | enantiomeric excess. |
| 4 | 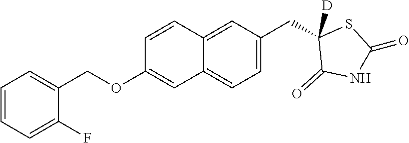 [SMILES: [2H][C@@]1(Cc2ccc3cc(OCc4ccccc4F)ccc3c2)SC(=O)NC1=O] |
| | having a stereochemical purity of at |
| | least 75% enantiomeric excess. |
| 5 | 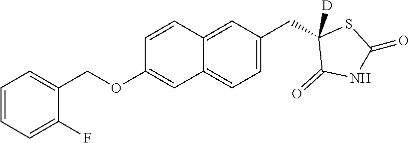 [SMILES: [2H][C@@]1(Cc2ccc3cc(OCc4ccccc4F)ccc3c2)SC(=O)NC1=O] |
| | or a pharmaceutically acceptable |
| | salt thereof, each having a stereochemical purity of at least 90% |
| | enantiomeric excess. |
| 6 | 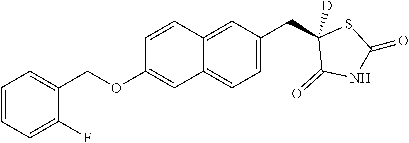 [SMILES: [2H][C@]1(Cc2ccc3cc(OCc4ccccc4F)ccc3c2)SC(=O)NC1=O] |
| | having a stereochemical purity of |
| | at least 90% enantiomeric excess. |
| 7 | 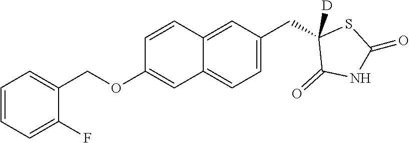 [SMILES: [2H][C@@]1(Cc2ccc3cc(OCc4ccccc4F)ccc3c2)SC(=O)NC1=O] |
| | or a pharmaceutically acceptable |
| | salt thereof, each having a stereochemical purity of at least 95% |
| | enantiomeric excess. |
| 8 | 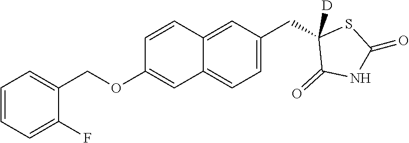 [SMILES: [2H][C@@]1(Cc2ccc3cc(OCc4ccccc4F)ccc3c2)SC(=O)NC1=O] |
| | having a stereochemical purity of at |
| | least 95% enantiomeric excess. |

TABLE 28

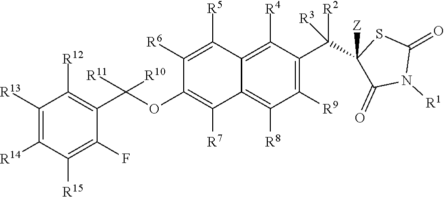

| Compound | |
|---|---|
| No. | Variable Definition |
| 1 | Z = D; R¹ = H; R²-R⁹ are D; and R¹⁰-R¹⁵ are H |
| 2 | Z = D; R¹ = H; R² and R³ are D; and R⁴-R¹⁵ are H |
| 3 | Z = D; R¹-R³ are H; R⁴-R⁹ are D; and R¹⁰-R¹⁵ are H |
| 4 | Z = D; R¹-R¹¹ are H; and R¹²-R¹⁵ are D |
| 5 | Z = D; R¹-R⁹ are H; R¹⁰ and R¹¹ are D; and R¹²-R¹⁵ are H |
| 6 | Z = D; R¹ = H; R² and R³ are D; R⁴-R⁹ are H; and R¹⁰-R¹⁵ are D |

Another embodiment provides a compound in Table 28 wherein the compound has an enantiomeric excess of at least 60%, 70%, 75%, 80%, 85%, 90%, 95% or 98%.

### Deuterium-Enriched Rivoglitazone

Another collection of deuterium-enriched compounds is represented by Formula I-H having a stereochemical purity of at least 75% enantiomeric excess:

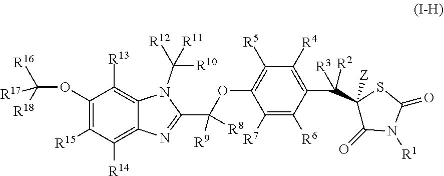

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are independently H or D.

In certain embodiments, R² and R³ are H. In certain embodiments, R⁸ and R⁹ are H. In certain embodiments, R⁴, R⁵, R⁶, and R⁷ are H. In certain embodiments, R¹³, R¹⁴, and R¹⁵ are H. In certain embodiments, R¹⁰, R¹¹, and R¹² are H. In certain other embodiments, R², R³, R⁸, and R⁹ are H.

In certain embodiments, R², R³, R¹³, R¹⁴, and R¹⁵ are H. In certain embodiments, R⁴, R⁵, R⁶, R⁷, R⁸, and R⁹ are H. In certain embodiments, R⁴, R⁵, R⁷, R⁸, R⁹, R¹³, R¹⁴, and R¹⁵ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain other embodiments, R¹, R², R³, R⁴, R⁵, R⁶, and R⁷ are H.

The description above describes multiple embodiments relating to compounds of Formula I-H. The patent application specifically contemplates all combinations of the embodiments. For example, the invention contemplates a compound of Formula I-H wherein R², R³, R⁴, R⁵, R⁶, R⁷, R⁸, and R⁹ are H.

Another collection of deuterium-enriched compounds is represented by Formula I-H1 having a stereochemical purity of at least 75% enantiomeric excess:

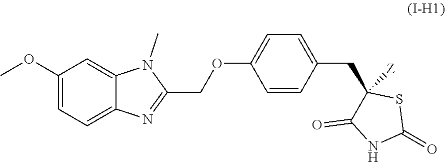

or a pharmaceutically acceptable salt thereof, wherein Z is H or D, provided that the abundance of deuterium in Z is at least 30%.

The compounds of Formula I-H and Formula I-H1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula I-H and Formula I-H1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 29 and 30 below.

TABLE 29

| Compound | |
|---|---|
| No. | Structure |
| 1 | 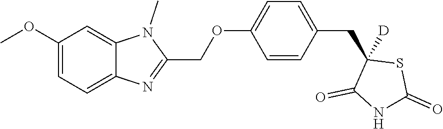 [SMILES: [2H][C@]1(Cc2ccc(OCc3nc4ccc(OC)cc4n3C)cc2)SC(=O)NC1=O] |
| | or a pharmaceutically |
| | acceptable salt thereof, each having a stereochemical purity of at least 60% |
| | enantiomeric excess. |
| 2 | 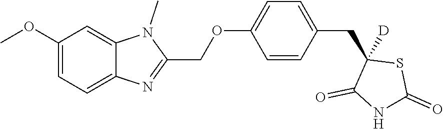 [SMILES: [2H][C@]1(Cc2ccc(OCc3nc4ccc(OC)cc4n3C)cc2)SC(=O)NC1=O] |
| | having a stereochemical purity |
| | of at least 60% enantiomeric excess. |
| 3 | 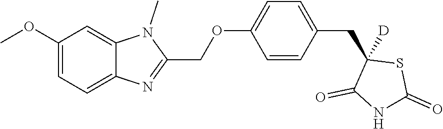 [SMILES: [2H][C@]1(Cc2ccc(OCc3nc4ccc(OC)cc4n3C)cc2)SC(=O)NC1=O] |
| | or a pharmaceutically |
| | acceptable salt thereof, each having a stereochemical purity of at least 75% |
| | enantiomeric excess. |
| 4 | 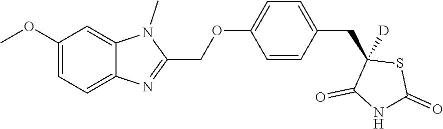 [SMILES: [2H][C@]1(Cc2ccc(OCc3nc4ccc(OC)cc4n3C)cc2)SC(=O)NC1=O] |
| | having a stereochemical purity |
| | of at least 75% enantiomeric excess. |
| 5 | 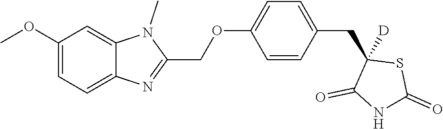 [SMILES: [2H][C@]1(Cc2ccc(OCc3nc4ccc(OC)cc4n3C)cc2)SC(=O)NC1=O] |
| | or a pharmaceutically |
| | acceptable salt thereof, each having a stereochemical purity of at least 90% |
| | enantiomeric excess. |
| 6 | 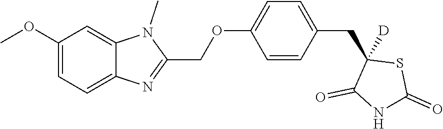 [SMILES: [2H][C@]1(Cc2ccc(OCc3nc4ccc(OC)cc4n3C)cc2)SC(=O)NC1=O] |
| | having a stereochemical purity |
| | of at least 90% enantiomeric excess. |
| 7 | 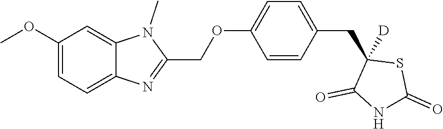 [SMILES: [2H][C@]1(Cc2ccc(OCc3nc4ccc(OC)cc4n3C)cc2)SC(=O)NC1=O] |
| | or a pharmaceutically |
| | acceptable salt thereof, each having a stereochemical purity of at least 95% |
| | enantiomeric excess. |
| 8 | 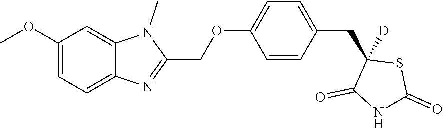 [SMILES: [2H][C@]1(Cc2ccc(OCc3nc4ccc(OC)cc4n3C)cc2)SC(=O)NC1=O] |
| | having a stereochemical purity |
| | of at least 95% enantiomeric excess. |

TABLE 30

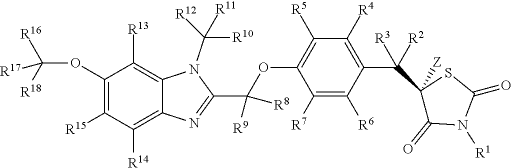

| Compound | |
|---|---|
| No. | Variable Definition |
| 1 | Z = D; R¹ = H; R²-R⁹ are D; and R¹⁰-R¹⁸ are H |
| 2 | Z = D; R¹ = H; R² and R³ are D; and R⁴-R¹⁸ are H |
| 3 | Z = D; R¹-R³ are H; R⁴-R⁷ are D; and R⁸-R¹⁸ are H |
| 4 | Z = D; R¹-R⁹ are H; and R¹⁰-R¹⁸ are D |
| 5 | Z = D; R¹-R⁷ are H; R⁸ and R⁹ are D; and R¹⁰-R¹⁸ are H |
| 6 | Z = D; R¹ = H; R² and R³ are D; R⁴-R⁷ are H; R⁸ and R⁹ are D; R¹⁰-R¹² are H; |
| | and R¹³-R¹⁸ are D |

Another embodiment provides a compound in Table 34 wherein the compound has an enantiomeric excess of at least 60%, 70%, 75%, 80%, 85%, 90%, 95%, or 98%.

Another collection of deuterium-enriched compounds is represented by Formula II-H having a stereochemical purity of at least 75% enantiomeric excess:

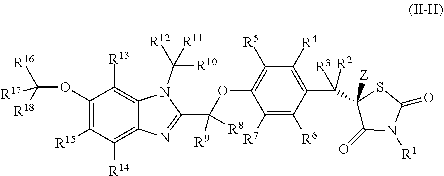

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are independently H or D.

In certain embodiments, R² and R³ are H. In certain embodiments, R⁸ and R⁹ are H. In certain embodiments, R⁴, R⁵, R⁶, and R⁷ are H. In certain embodiments, R¹³, R¹⁴, and R¹⁵ are H. In certain embodiments, R¹⁰, R¹¹, and R¹² are H. In certain other embodiments, R², R³, R⁸, and R⁹ are H.

In certain embodiments, R², R³, R¹³, R¹⁴, and R¹⁵ are H. In certain embodiments, R⁴, R⁵, R⁶, R⁷, R⁸, and R⁹ are H. In certain embodiments, R⁴, R⁵, R⁶, R⁷, R⁹, R¹³, R¹⁴, and R¹⁵ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain other embodiments, R¹, R², R³, R⁴, R⁵, R⁶, and R⁷ are H.

The description above describes multiple embodiments relating to compounds of Formula II-H. The patent application specifically contemplates all combinations of the embodiments. For example, the invention contemplates a compound of Formula II-H wherein R², R³, R⁴, R⁵, R⁶, R⁷, R⁸, and R⁹ are H.

Another collection of deuterium-enriched compounds is represented by Formula II-H1 having a stereochemical purity of at least 75% enantiomeric excess:

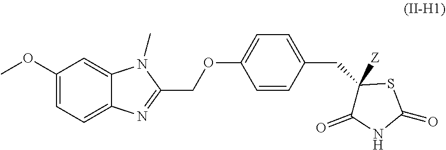

or a pharmaceutically acceptable salt thereof, wherein Z is H or D, provided that the abundance of deuterium in Z is at least 30%.

The compounds of Formula II-H and Formula II-H1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 10000.

In certain embodiments, the abundance of deuterium in Z is at least 600%. In certain other embodiments, the abundance of deuterium in Z is at least 7500 In yet other embodiments, the abundance of deuterium in Z is at least 9000.

The compounds of Formula II-H and Formula II-H1 can be further characterized according their enantiomeric purity. In certain embodiments, the deuterium-enriched compound has an enantiomeric excess of at least 80%, 85%, 90%, 95%, or 98%. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 990.

Still further such deuterium-enriched compounds are provided in Tables 31 and 32 below.

TABLE 31

| Compound | |
|---|---|
| No. | Structure |
| 1 | 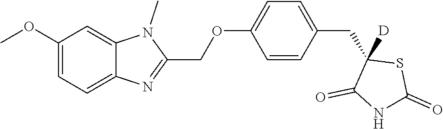 [SMILES: [2H][C@@]1(Cc2ccc(OCc3nc4ccc(OC)cc4n3C)cc2)SC(=O)NC1=O] |
| | or a pharmaceutically |
| | acceptable salt thereof, each having a stereochemical purity of at least 60% |
| | enantiomeric excess. |
| 2 | 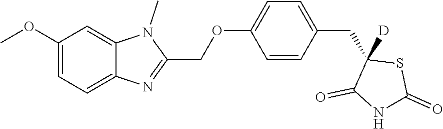 [SMILES: [2H][C@@]1(Cc2ccc(OCc3nc4ccc(OC)cc4n3C)cc2)SC(=O)NC1=O] |
| | having a stereochemical purity |
| | of at least 60% enantiomeric excess. |
| 3 | 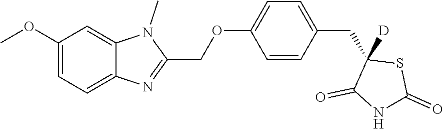 [SMILES: [2H][C@@]1(Cc2ccc(OCc3nc4ccc(OC)cc4n3C)cc2)SC(=O)NC1=O] |
| | or a pharmaceutically |
| | acceptable salt thereof, each having a stereochemical purity of at least 75% |
| | enantiomeric excess. |
| 4 | 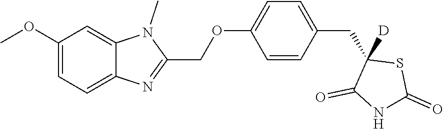 [SMILES: [2H][C@@]1(Cc2ccc(OCc3nc4ccc(OC)cc4n3C)cc2)SC(=O)NC1=O] |
| | having a stereochemical purity |
| | of at least 75% enantiomeric excess. |
| 5 | 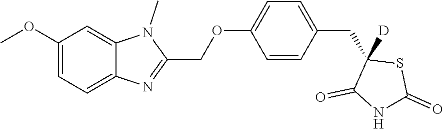 [SMILES: [2H][C@@]1(Cc2ccc(OCc3nc4ccc(OC)cc4n3C)cc2)SC(=O)NC1=O] |
| | or a pharmaceutically |
| | acceptable salt thereof, each having a stereochemical purity of at least 90% |
| | enantiomeric excess. |
| 6 | 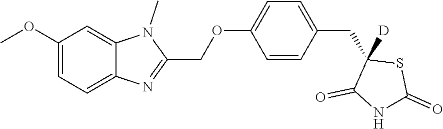 [SMILES: [2H][C@@]1(Cc2ccc(OCc3nc4ccc(OC)cc4n3C)cc2)SC(=O)NC1=O] |
| | having a stereochemical purity |
| | of at least 90% enantiomeric excess. |
| 7 | 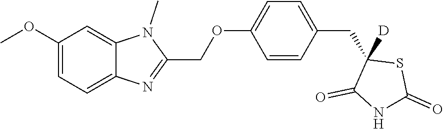 [SMILES: [2H][C@@]1(Cc2ccc(OCc3nc4ccc(OC)cc4n3C)cc2)SC(=O)NC1=O] |
| | or a pharmaceutically |
| | acceptable salt thereof, each having a stereochemical purity of at least 95% |
| | enantiomeric excess. |
| 8 | 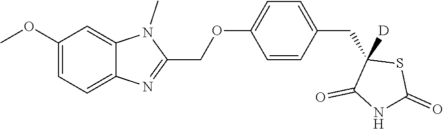 [SMILES: [2H][C@@]1(Cc2ccc(OCc3nc4ccc(OC)cc4n3C)cc2)SC(=O)NC1=O] |
| | having a stereochemical purity |
| | of at least 95% enantiomeric excess. |

TABLE 32

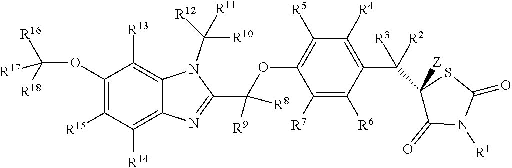

| Compound | |
|---|---|
| No. | Variable Definition |
| 1 | Z = D; R¹ = H; R²-R⁹ are D; and R¹⁰-R¹⁸ are H |
| 2 | Z = D; R¹ = H; R² and R³ are D; and R⁴-R¹⁸ are H |
| 3 | Z = D; R¹-R³ are H; R⁴-R⁷ are D; and R⁸-R¹⁸ are H |
| 4 | Z = D; R¹-R⁹ are H; and R¹⁰-R¹⁸ are D |
| 5 | Z = D; R¹-R⁷ are H; R⁸ and R⁹ are D; and R¹⁰-R¹⁸ are H |
| 6 | Z = D; R¹ = H; R² and R³ are D; R⁴-R⁷ are H; R⁸ and R⁹ are D; R¹⁰-R¹² are H; |
| | and R¹³-R¹⁸ are D |

Another embodiment provides a compound in Table 32 wherein the compound has an enantiomeric excess of at least 60%, 70%, 75%, 80%, 85%, 90%, 95%, or 98%.

### Deuterium-Enriched Troglitazone

Another collection of deuterium-enriched compounds is represented by Formula I-I having a stereochemical purity of at least 75% enantiomeric excess at the carbon atom bearing variable Z:

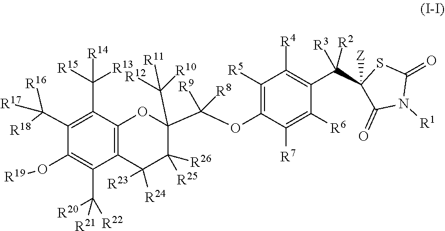

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, and R²⁶ are independently H or D.

In certain embodiments, R² and R³ are H. In certain embodiments, R⁸ and R⁹ are H. In certain embodiments, R⁴, R⁵, R⁶, and R⁷ are H. In certain embodiments, R¹³, R¹⁴, and R¹⁵ are H. In certain embodiments, R¹⁰, R¹¹, and R¹² are H. In certain other embodiments, R², R³, R⁸, and R⁹ are H. In certain embodiments, R¹ and R¹⁹ are H. In still other embodiments, R²³, R²⁴, R²⁵, and R²⁶ are H.

In certain embodiments, R², R³, R¹⁰, R¹¹, and R¹² are H. In certain embodiments, R⁴, R⁵, R⁶, R⁷, R⁸, and R⁹ are H. In certain embodiments, R⁴, R, R⁶, R⁷, R⁹, R¹³, R¹⁴, and R¹⁵ are H. In certain other embodiments, R¹³, R¹⁴, R¹⁵, R¹⁶, R¹⁷, R¹⁸, R¹⁹, R²⁰, R²¹, and R²² are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R¹⁹ is H. In certain other embodiments, R¹, R², R³, R⁴, R⁵, R⁶, and R⁷ are H.

The description above describes multiple embodiments relating to compounds of Formula I-I. The patent application specifically contemplates all combinations of the embodiments. For example, the invention contemplates a compound of Formula I-I wherein R², R³, R⁴, R⁵, R⁶, R⁷, R⁸, and R⁹ are H.

Another collection of deuterium-enriched compounds is represented by Formula I-I1 having a stereochemical purity of at least 75% enantiomeric excess at the carbon atom bearing variable Z:

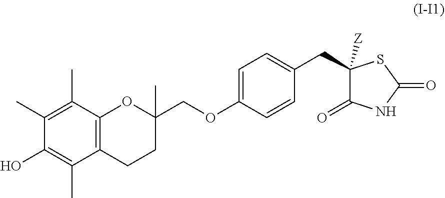

or a pharmaceutically acceptable salt thereof, wherein Z is H or D, provided that the abundance of deuterium in Z is at least 30%.

The compounds of Formula I-I and Formula I-I1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula I-I and Formula I-I1 can be further characterized according their enantiomeric purity. In certain embodiments, the deuterium-enriched compound has an enantiomeric excess of at least 80%, 85%, 90%, 95%, or 98% at the carbon atom bearing Z. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 33 and 34 below.

TABLE 33

| Compound | |
|---|---|
| No. | Structure |
| 1 | 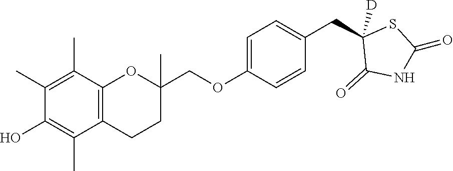 [SMILES: [2H][C@]1(Cc2ccc(OCC3(C)CCc4c(C)c(O)c(C)c(C)c4O3)cc2)SC(=O)NC1=O] |
| | or a pharmaceutically |
| | acceptable salt thereof, each having a stereochemical purity of at least 60% |
| | enantiomeric excess at the carbon atom bearing D. |
| 2 | 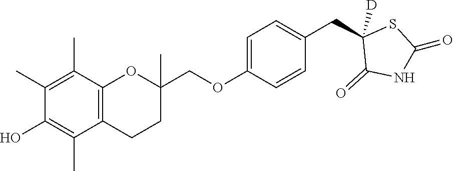 [SMILES: [2H][C@]1(Cc2ccc(OCC3(C)CCc4c(C)c(O)c(C)c(C)c4O3)cc2)SC(=O)NC1=O] |
| | having a stereochemical |
| | purity of at least 60% enantiomeric excess at the carbon atom bearing D. |
| 3 | 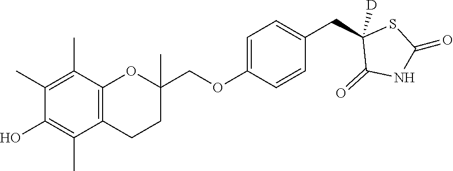 [SMILES: [2H][C@]1(Cc2ccc(OCC3(C)CCc4c(C)c(O)c(C)c(C)c4O3)cc2)SC(=O)NC1=O] |
| | or a pharmaceutically |
| | acceptable salt thereof, each having a stereochemical purity of at least 75% |
| | enantiomeric excess at the carbon atom bearing D. |
| 4 | 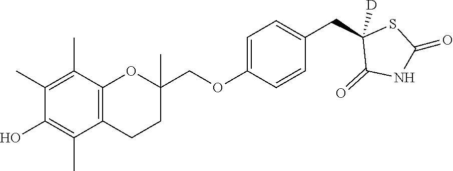 [SMILES: [2H][C@]1(Cc2ccc(OCC3(C)CCc4c(C)c(O)c(C)c(C)c4O3)cc2)SC(=O)NC1=O] |
| | having a stereochemical |
| | purity of at least 75% enantiomeric excess at the carbon atom bearing D. |
| 5 | 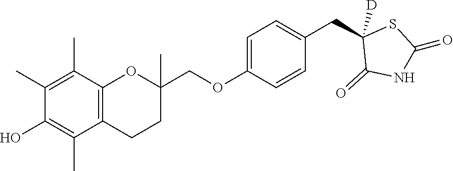 [SMILES: [2H][C@]1(Cc2ccc(OCC3(C)CCc4c(C)c(O)c(C)c(C)c4O3)cc2)SC(=O)NC1=O] |
| | or a pharmaceutically |
| | acceptable salt thereof, each having a stereochemical purity of at least 90% |
| | enantiomeric excess at the carbon atom bearing D. |
| 6 | 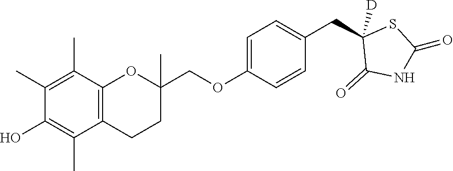 [SMILES: [2H][C@]1(Cc2ccc(OCC3(C)CCc4c(C)c(O)c(C)c(C)c4O3)cc2)SC(=O)NC1=O] |
| | having a stereochemical |
| | purity of at least 90% enantiomeric excess at the carbon atom bearing D. |
| 7 | 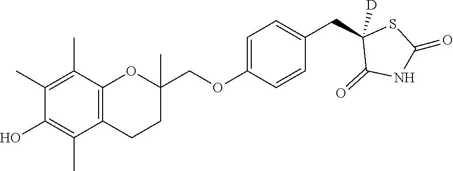 [SMILES: [2H][C@]1(Cc2ccc(OCC3(C)CCc4c(C)c(O)c(C)c(C)c4O3)cc2)SC(=O)NC1=O] |
| | or a pharmaceutically |
| | acceptable salt thereof, each having a stereochemical purity of at least 95% |
| | enantiomeric excess at the carbon atom bearing D. |
| 8 | 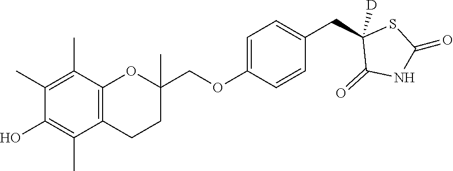 [SMILES: [2H][C@]1(Cc2ccc(OCC3(C)CCc4c(C)c(O)c(C)c(C)c4O3)cc2)SC(=O)NC1=O] |
| | having a stereochemical |
| | purity of at least 95% enantiomeric excess at the carbon atom bearing D. |

TABLE 34

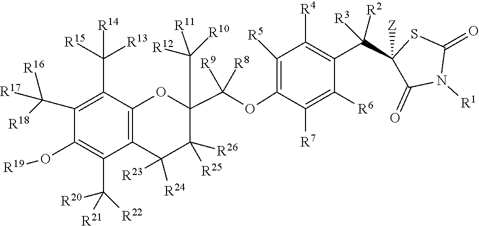

| Compound | |
|---|---|
| No. | Variable Definition |
| 1 | Z = D; R¹ = H; R²-R⁹ are D; and R¹⁰-R²⁶ are H |
| 2 | Z = D; R¹ = H; R² and R³ are D; and R⁴-R²⁶ are H |
| 3 | Z = D; R¹-R³ are H; R⁴-R⁷ are D; and R⁸-R²⁶ are H |
| 4 | Z = D; R¹-R⁹ are H; R¹⁰-R¹⁸ are D; R¹⁹ = H; and R²⁰-R²⁶ are H |
| 5 | Z = D; R¹-R⁷ are H; R⁸ and R⁹ are D; and R¹⁰-R²⁶ are H |
| 6 | Z = D; R¹ = H; R² and R³ are D; R⁴-R⁷ are H; R⁸ and R⁹ are D; R¹⁰-R²² are H; |
| | and R²³-R²⁶ are D |

Another embodiment provides a compound in Table 34 wherein the compound has an enantiomeric excess of at least 60%, 70%, 75%, 80%, 85%, 90%, 95%, or 98%.

Another collection of deuterium-enriched compounds is represented by Formula II-I having a stereochemical purity of at least 75% enantiomeric excess at the carbon atom bearing variable Z:

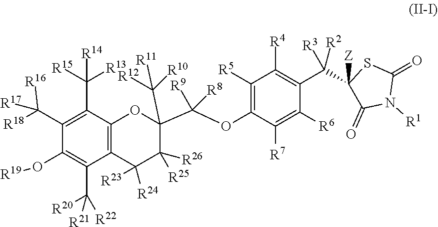

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, and R²⁶ are independently H or D.

In certain embodiments, R² and R³ are H. In certain embodiments, R⁸ and R⁹ are H. In certain embodiments, R⁴, R⁵, R⁶, and R⁷ are H. In certain embodiments, R¹³, R¹⁴, and R¹⁵ are H. In certain embodiments, R¹⁰, R¹¹, and R¹² are H. In certain other embodiments, R², R³, R⁸, and R⁹ are H. In certain embodiments, R¹ and R¹⁹ are H. In still other embodiments, R²³, R²⁴, R²⁵, and R²⁶ are H.

In certain embodiments, R², R³, R¹⁰, R¹¹, and R¹² are H. In certain embodiments, R⁴, R⁵, R⁶, R⁷, R⁸, and R⁹ are H. In certain embodiments, R⁴, R⁵, R⁶, R⁷, R⁸, R⁹, R¹³, R¹⁴, and R¹⁵ are H. In certain other embodiments, R¹³, R¹⁴, R¹⁵, R¹⁶, R¹⁷, R¹⁸, R¹⁹, R²⁰, R²¹, and R²² are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R¹⁹ is H. In certain other embodiments, R¹, R², R³, R⁴, R⁵, R⁶, and R⁷ are H.

The description above describes multiple embodiments relating to compounds of Formula II-I. The patent application specifically contemplates all combinations of the embodiments. For example, the invention contemplates a compound of Formula II-I wherein R², R³, R⁴, R⁵, R⁶, R⁷, R⁸, and R⁹ are H.

Another collection of deuterium-enriched compounds represented by Formula II-I1 having a stereochemical purity of at least 75% enantiomeric excess at the carbon atom bearing variable Z:

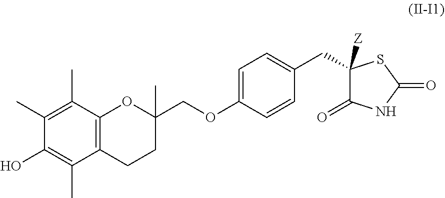

or a pharmaceutically acceptable salt thereof, wherein Z is H or D, provided that the abundance of deuterium in Z is at least 30%.

The compounds of Formula II-I and Formula II-I1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula II-I and Formula II-I1 can be further characterized according their enantiomeric purity. In certain embodiments, the deuterium-enriched compound has an enantiomeric excess of at least 80%, 85%, 90%, 95%, or 98% at the carbon atom bearing variable Z. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 35 and 36 below.

TABLE 35

| Compound | |
|---|---|
| No. | Structure |
| 1 | 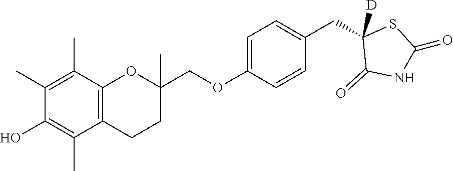 [SMILES: [2H][C@@]1(Cc2ccc(OCC3(C)CCc4c(C)c(O)c(C)c(C)c4O3)cc2)SC(=O)NC1=O] |
| | or a pharmaceutically |
| | acceptable salt thereof, each having a stereochemical purity of at least 60% |
| | enantiomeric excess at the carbon atom bearing D. |
| 2 | 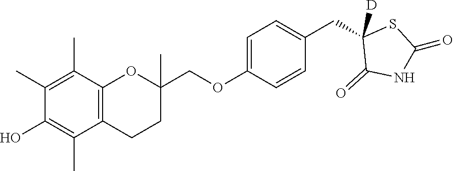 [SMILES: [2H][C@@]1(Cc2ccc(OCC3(C)CCc4c(C)c(O)c(C)c(C)c4O3)cc2)SC(=O)NC1=O] |
| | having a stereochemical |
| | purity of at least 60% enantiomeric excess at the carbon atom bearing D. |
| 3 | 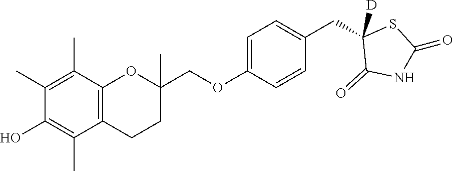 [SMILES: [2H][C@@]1(Cc2ccc(OCC3(C)CCc4c(C)c(O)c(C)c(C)c4O3)cc2)SC(=O)NC1=O] |
| | or a pharmaceutically |
| | acceptable salt thereof, each having a stereochemical purity of at least 75% |
| | enantiomeric excess at the carbon atom bearing D. |
| 4 | 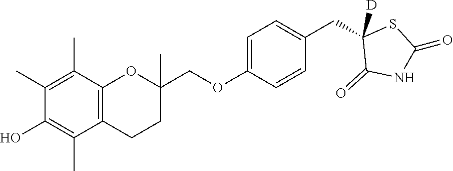 [SMILES: [2H][C@@]1(Cc2ccc(OCC3(C)CCc4c(C)c(O)c(C)c(C)c4O3)cc2)SC(=O)NC1=O] |
| | having a stereochemical |
| | purity of at least 75% enantiomeric excess at the carbon atom bearing D. |
| 5 | 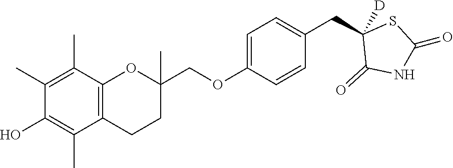 [SMILES: [2H][C@]1(Cc2ccc(OCC3(C)CCc4c(C)c(O)c(C)c(C)c4O3)cc2)SC(=O)NC1=O] |
| | or a pharmaceutically |
| | acceptable salt thereof, each having a stereochemical purity of at least 90% |
| | enantiomeric excess at the carbon atom bearing D. |
| 6 | 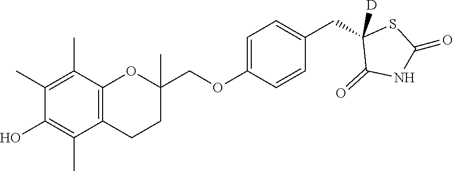 [SMILES: [2H][C@@]1(Cc2ccc(OCC3(C)CCc4c(C)c(O)c(C)c(C)c4O3)cc2)SC(=O)NC1=O] |
| | having a stereochemical |
| | purity of at least 90% enantiomeric excess at the carbon atom bearing D. |
| 7 | 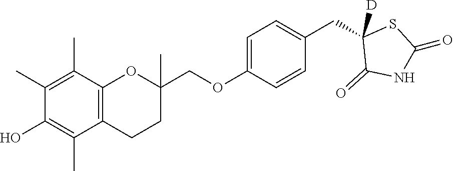 [SMILES: [2H][C@@]1(Cc2ccc(OCC3(C)CCc4c(C)c(O)c(C)c(C)c4O3)cc2)SC(=O)NC1=O] |
| | or a pharmaceutically |
| | acceptable salt thereof, each having a stereochemical purity of at least 95% |
| | enantiomeric excess at the carbon atom bearing D. |
| 8 | 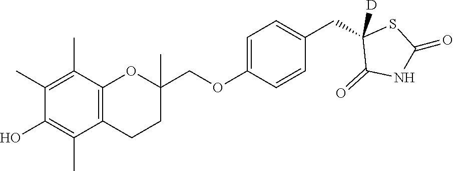 [SMILES: [2H][C@@]1(Cc2ccc(OCC3(C)CCc4c(C)c(O)c(C)c(C)c4O3)cc2)SC(=O)NC1=O] |
| | having a stereochemical |
| | purity of at least 95% enantiomeric excess at the carbon atom bearing D. |

TABLE 36

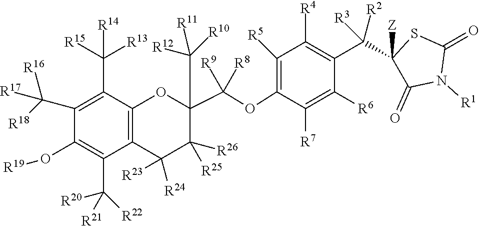

| Compound | |
|---|---|
| No. | Variable Definition |
| 1 | Z = D; R¹ = H; R²-R⁹ are D; and R¹⁰-R²⁶ are H |
| 2 | Z = D; R¹ = H; R² and R³ are D; and R⁴-R²⁶ are H |
| 3 | Z = D; R¹-R³ are H; R⁴-R⁷ are D; and R⁸-R²⁶ are H |
| 4 | Z = D; R¹-R⁹ are H; R¹⁰-R¹⁸ are D; R¹⁹ = H; and R²⁰-R²⁶ are H |
| 5 | Z = D; R¹-R⁷ are H; R⁸ and R⁹ are D; and R¹⁰-R²⁶ are H |
| 6 | Z = D; R¹ = H; R² and R³ are D; R⁴-R⁷ are H; R⁸ and R⁹ are D; R¹⁰-R²² are H; |
| | and R²³-R²⁶ are D |

Another embodiment provides a compound in Table 36 wherein the compound has an enantiomeric excess of at least 60%, 70%, 75%, 80%, 85%, 90%, 95%, or 98%.

### Deuterium-Enriched Rosiglitazone

Another collection of deuterium-enriched compounds is represented by Formula I-J having a stereochemical purity of at least 75% enantiomeric excess at the carbon atom bearing variable Z:

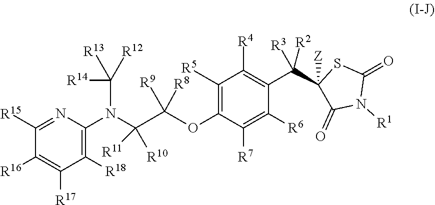

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are independently H or D.

In certain embodiments, R² and R³ are H. In certain embodiments, R⁸ and R⁹ are H. In certain embodiments, R⁴, R⁵, R⁶, and R⁷ are H. In certain embodiments, R⁸, R⁹, R¹⁰, and R¹¹ are H. In certain embodiments, R¹², R¹³, and R¹⁴ are H. In certain other embodiments, R², R³, R⁸, and R⁹ are H.

In certain embodiments, R², R³, R⁸, R⁹, R¹⁰, and R¹¹ are H. In certain embodiments, R⁴, R⁵, R⁶, R⁷, R⁸, and R⁹ are H. In certain embodiments, R⁴, R⁵, R⁶, R⁷, R⁸, R¹⁶, R¹⁷, and R¹⁸ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, R⁸ and R⁹ are H.

The description above describes multiple embodiments relating to compounds of Formula I-J. The patent application specifically contemplates all combinations of the embodiments. For example, the invention contemplates a compound of Formula I-J wherein R², R³, R⁴, R⁵, R⁶, R⁷, R⁸, R⁹, R¹⁰, and R¹¹ are H.

Another collection of deuterium-enriched compounds is represented by Formula I-J1 having a stereochemical purity of at least 75% enantiomeric excess:

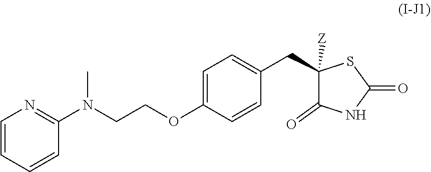

or a pharmaceutically acceptable salt thereof, wherein Z is H or D, provided that the abundance of deuterium in Z is at least 30%.

The compounds of Formula I-J and Formula I-J1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula I-J and Formula I-J1 can be further characterized according their enantiomeric purity. In certain embodiments, the deuterium-enriched compound has an enantiomeric excess of at least 80%, 85%, 90%, 95%, or 98% at the carbon atom bearing variable Z. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 37 and 38 below.

TABLE 37

| Compound | |
|---|---|
| No. | Structure |
| 1 | 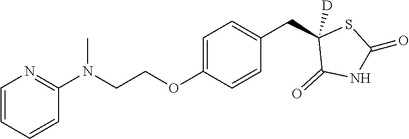 [SMILES: [2H][C@]1(Cc2ccc(OCCN(C)c3ccccn3)cc2)SC(=O)NC1=O] |
| | or a pharmaceutically acceptable |
| | salt thereof, each having a stereochemical purity of at least 60% |
| | enantiomeric excess. |
| 2 | 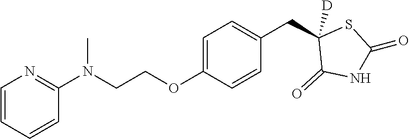 [SMILES: [2H][C@]1(Cc2ccc(OCCN(C)c3ccccn3)cc2)SC(=O)NC1=O] |
| | having a stereochemical purity of at |
| | least 60% enantiomeric excess. |
| 3 | 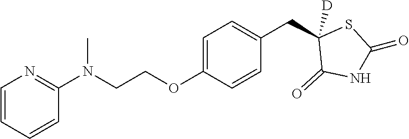 [SMILES: [2H][C@]1(Cc2ccc(OCCN(C)c3ccccn3)cc2)SC(=O)NC1=O] |
| | or a pharmaceutically acceptable |
| | salt thereof, each having a stereochemical purity of at least 75% |
| | enantiomeric excess. |
| 4 | 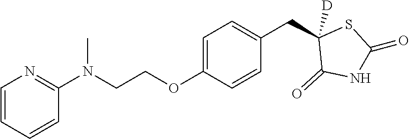 [SMILES: [2H][C@]1(Cc2ccc(OCCN(C)c3ccccn3)cc2)SC(=O)NC1=O] |
| | having a stereochemical purity of at |
| | least 75% enantiomeric excess. |
| 5 | 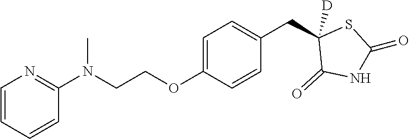 [SMILES: [2H][C@]1(Cc2ccc(OCCN(C)c3ccccn3)cc2)SC(=O)NC1=O] |
| | or a pharmaceutically acceptable |
| | salt thereof, each having a stereochemical purity of at least 90% |
| | enantiomeric excess. |
| 6 | 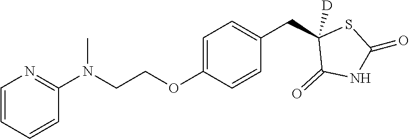 [SMILES: [2H][C@]1(Cc2ccc(OCCN(C)c3ccccn3)cc2)SC(=O)NC1=O] |
| | having a stereochemical purity of at |
| | least 90% enantiomeric excess. |
| 7 | 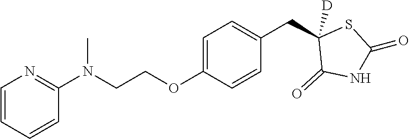 [SMILES: [2H][C@]1(Cc2ccc(OCCN(C)c3ccccn3)cc2)SC(=O)NC1=O] |
| | or a pharmaceutically acceptable |
| | salt thereof, each having a stereochemical purity of at least 95% |
| | enantiomeric excess. |
| 8 | 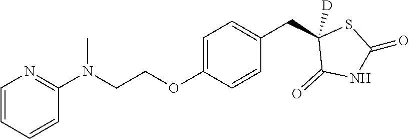 [SMILES: [2H][C@]1(Cc2ccc(OCCN(C)c3ccccn3)cc2)SC(=O)NC1=O] |
| | having a stereochemical purity of at |
| | least 95% enantiomeric excess. |

TABLE 38

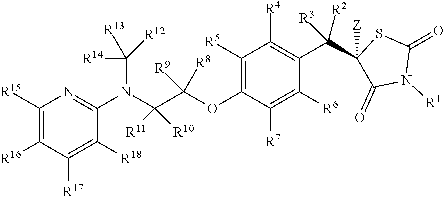

| Compound | |
|---|---|
| No. | Variable Definition |
| 1 | Z = D; R¹ = H; R²-R⁹ are D; and R¹⁰-R¹⁸ are H |
| 2 | Z = D; R¹ = H; R² and R³ are D; and R⁴-R¹⁸ are H |
| 3 | Z = D; R¹-R³ are H; R⁴-R⁷ are D; and R⁸-R¹⁸ are H |
| 4 | Z = D; R¹-R⁷ are H; R⁸-R¹⁴ are D; and R¹⁵-R¹⁸ are H |
| 5 | Z = D; R¹-R⁹ are H; R¹⁰-R¹¹ are D; and R¹²-R¹⁸ are H |
| 6 | Z = D; R¹ = H; R² and R³ are D; R⁴-R⁷ are H; R⁸-R¹¹ are D; and R¹²-R¹⁸ are H |

Another embodiment provides a compound in Table 38 wherein the compound has an enantiomeric excess of at least 60%, 70%, 75%, 80%, 85%, 90%, 95% or 98%.

Another collection of deuterium-enriched compounds is represented by Formula II-J having a stereochemical purity of at least 75% enantiomeric excess at the carbon atom bearing variable Z:

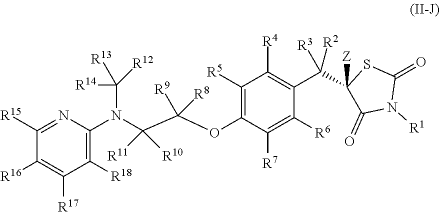

or a pharmaceutically acceptable salt thereof, wherein:
Z is H or D, provided that the abundance of deuterium in Z is at least 30%; and
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are independently H or D.

In certain embodiments, R² and R³ are H. In certain embodiments, R⁸ and R⁹ are H. In certain embodiments, R⁴, R⁵, R⁶, and R⁷ are H. In certain embodiments, R⁸, R⁹, R¹⁰, and R¹¹ are H. In certain embodiments, R¹², R¹³, and R¹⁴ are H. In certain other embodiments, R², R³, R⁸, and R⁹ are H.

In certain embodiments, R², R³, R⁸, R⁹, R¹⁰, and R¹¹ are H. In certain embodiments, R⁴, R⁵, R⁶, R⁷, R⁸, and R⁹ are H. In certain embodiments, R⁴, R⁵, R⁶, R⁷, R⁸, R¹⁵, R¹⁶, R¹⁷, and R¹⁸ are H.

In certain embodiments, R¹ is H. In certain embodiments, R² is H. In certain embodiments, R³ is H. In certain embodiments, R⁴ is H. In certain embodiments, R⁵ is H. In certain embodiments, R⁶ is H. In certain embodiments, R⁷ is H. In certain embodiments, R⁸ is H. In certain other embodiments, R¹, R², R³, R⁴, R⁵, R⁶, R⁷, R⁸ and R⁹ are H.

The description above describes multiple embodiments relating to compounds of Formula II-J. The patent application specifically contemplates all combinations of the embodiments. For example, the invention contemplates a compound of Formula II-J wherein R², R³, R⁴, R⁵, R⁶, R⁷, R⁸, R⁹, R¹⁰, and R¹¹ are H.

Another collection of deuterium-enriched compounds is represented by Formula II-J1 having a stereochemical purity of at least 75% enantiomeric excess:

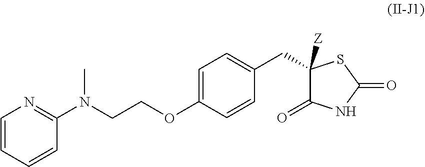

or a pharmaceutically acceptable salt thereof, wherein Z is H or D, provided that the abundance of deuterium in Z is at least 30%.

The compounds of Formula II-J and Formula II-J1 can be further characterized according to the abundance of deuterium at the position defined by variable Z. In certain embodiments, the abundance of deuterium in Z is selected from: (a) at least 40%, (b) at least 50%, (c) at least 60%, (d) at least 70%, (e) at least 75%, (f) at least 80%, (g) at least 90%, (h) at least 95%, (h) at least 97%, and (i) about 100%. Additional examples of the abundance of deuterium in Z include 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100%.

In certain embodiments, the abundance of deuterium in Z is at least 60%. In certain other embodiments, the abundance of deuterium in Z is at least 75%. In yet other embodiments, the abundance of deuterium in Z is at least 90%.

The compounds of Formula II-J and Formula II-J1 can be further characterized according their enantiomeric purity. In certain embodiments, the deuterium-enriched compound has an enantiomeric excess of at least 80%, 85%, 90%, 95%, or 98% at the carbon atom bearing variable Z. Still further examples of the stereochemical purity include an enantiomeric excess of at least 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

Still further such deuterium-enriched compounds are provided in Tables 39 and 40 below.

TABLE 39

| Compound | |
|---|---|
| No. | Structure |
| 1 | 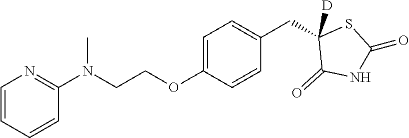 [SMILES: [2H][C@@]1(Cc2ccc(OCCN(C)c3ccccn3)cc2)SC(=O)NC1=O] |
| | or a pharmaceutically acceptable |
| | salt thereof, each having a stereochemical purity of at least 60% |
| | enantiomeric excess. |
| 2 | 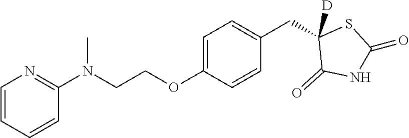 [SMILES: [2H][C@@]1(Cc2ccc(OCCN(C)c3ccccn3)cc2)SC(=O)NC1=O] |
| | having a stereochemical purity of at |
| | least 60% enantiomeric excess. |
| 3 | 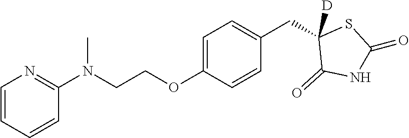 [SMILES: [2H][C@@]1(Cc2ccc(OCCN(C)c3ccccn3)cc2)SC(=O)NC1=O] |
| | or a pharmaceutically acceptable |
| | salt thereof, each having a stereochemical purity of at least 75% |
| | enantiomeric excess. |
| 4 | 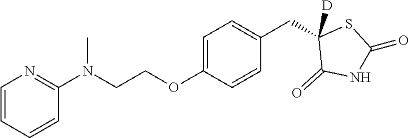 [SMILES: [2H][C@@]1(Cc2ccc(OCCN(C)c3ccccn3)cc2)SC(=O)NC1=O] |
| | having a stereochemical purity of at |
| | least 75% enantiomeric excess. |
| 5 | 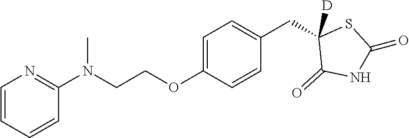 [SMILES: [2H][C@@]1(Cc2ccc(OCCN(C)c3ccccn3)cc2)SC(=O)NC1=O] |
| | or a pharmaceutically acceptable |
| | salt thereof, each having a stereochemical purity of at least 90% |
| | enantiomeric excess. |
| 6 | 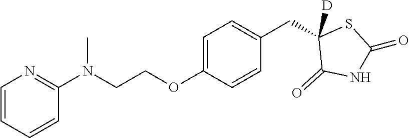 [SMILES: [2H][C@@]1(Cc2ccc(OCCN(C)c3ccccn3)cc2)SC(=O)NC1=O] |
| | having a stereochemical purity of at |
| | least 90% enantiomeric excess. |
| 7 | 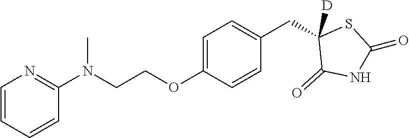 [SMILES: [2H][C@@]1(Cc2ccc(OCCN(C)c3ccccn3)cc2)SC(=O)NC1=O] |
| | or a pharmaceutically acceptable |
| | salt thereof, each having a stereochemical purity of at least 95% |
| | enantiomeric excess. |
| 8 | 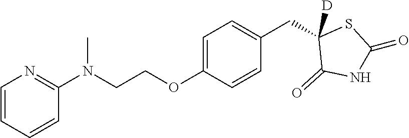 [SMILES: [2H][C@@]1(Cc2ccc(OCCN(C)c3ccccn3)cc2)SC(=O)NC1=O] |
| | having a stereochemical purity of at |
| | least 95% enantiomeric excess. |

TABLE 40

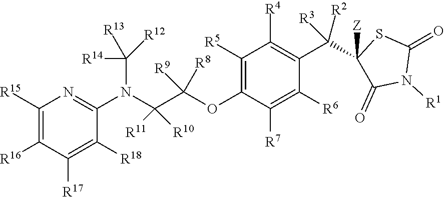

| Compound | |
|---|---|
| No. | Variable Definition |
| 1 | Z = D; R¹ = H; R²-R⁹ are D; and R¹⁰-R¹⁸ are H |
| 2 | Z = D; R¹ = H; R² and R³ are D; and R⁴-R¹⁸ are H |
| 3 | Z = D; R¹-R³ are H; R⁴-R⁷ are D; and R⁸-R¹⁸ are H |
| 4 | Z = D; R¹-R⁷ are H; R⁸-R¹⁴ are D; and R¹⁵-R¹⁸ are H |
| 5 | Z = D; R¹-R⁹ are H; R¹⁰-R¹¹ are D; and R¹²-R¹⁸ are H |
| 6 | Z = D; R¹ = H; R² and R³ are D; R⁴-R⁷ are H; R⁸-R¹¹ are D; and R¹²-R¹⁸ are H |

Another embodiment provides a compound in Table 40 wherein the compound has an enantiomeric excess of at least 60%, 70%, 75%, 80%, 85%, 90%, 95%, or 98%.

As indicated above, the deuterium-enriched compounds described above may be in the form of a pharmaceutically acceptable salt. One such pharmaceutically acceptable salt is a hydrochloride salt.

It is understood that the deuterium-enriched compounds described herein can be combined with a pharmaceutically acceptable carrier to form a pharmaceutical composition.

Deuterium-enriched compounds of the invention can generally be prepared by substituting a deuterium-enriched reagent for a non-isotopically labeled reagent in synthetic schemes reported in the literature for making non-isotopically labeled glitazone. Scheme 1 below illustrates a general method for preparing compounds having deuterium enrichment at the position defined by variable Z in Formula I. The scheme is provided for the purpose of illustrating the invention, and should not be regarded in any manner as limiting the scope or the spirit of the invention. In Scheme 1, compound A is first stirred with perdeuterated dimethylsulfoxide (d₆-DMSO) and triethylamine and then treated with perdeuterated methanol (d₄-MeOH). The R-enantiomer and S-enantiomer of deutero-thiazolidine B are separated using chiral chromatography, such as chiral high-performance liquid chromatography, to produce the deuterium-enriched compounds in stereochemically pure form. Alternatively, the R-enantiomer and S-enantiomer of deutero-thiazolidine B may be separated by reaction with a chiral resolving agent, followed by separation of the resulting diastereomers, and conversion back to deuterated glitazone in enantiopure form.

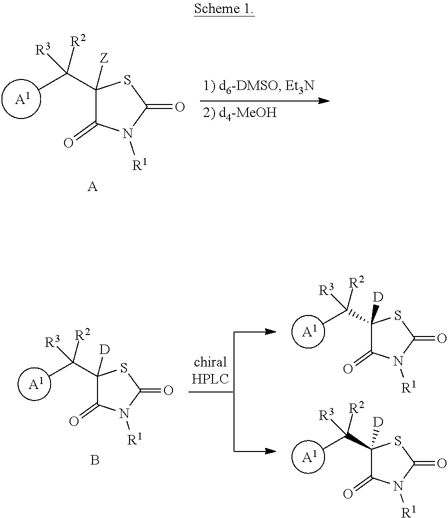

Compounds having deuterium enrichment at a position other than the position defined by variable Z in Formula I can be prepared by using deuterium-labeled starting materials to prepare compound A or introducing deuterium into compound A during its preparation.

Compounds described herein can be provided in isolated or purified form. Isolated or purified compounds are a group of compounds that have been separated from their environment, such as from a crude reaction mixture if made in a laboratory setting or removed from their natural environment if naturally occurring. Examples of the purity of the isolated compound include, for example, at least 50%, 55%, 60%, 65%, 70%, 75%, 80%, 85%, 90%, 95%, or 99% by weight.

Another aspect of the invention provides a unit quantum of a deuterium-enriched compound described herein, such as an amount of at least (a) one Dg of a disclosed deuterium-enriched compound, (b) one mg, or (c) one gram. In further embodiments, the quantum is, for example, at least 0.01, 0.02, 0.03, 0.04, 0.05, 0.1, 0.2, 0.3, 0.4, 0.5, or 1 mole of the compound. The present amounts also cover lab-scale (e.g., gram scale including 1, 2, 3, 4, 5 g, etc.), kilo-lab scale (e.g., kilogram scale including 1, 2, 3, 4, 5 kg, etc.), and industrial or commercial scale (e.g., multi-kilogram or above scale including 100, 200, 300, 400, 500 kg, etc.) quantities as these will be more useful in the actual manufacture of a pharmaceutical. Industrial/commercial scale refers to the amount of product that would be produced in a batch that was designed for clinical testing, formulation, sale/distribution to the public, etc.

### II. Therapeutic Applications

The invention provides methods of using deuterium-enriched compounds described herein to treat medical disorders. Preferred medical disorders for treatment include metabolic disorders, neurological disorders, cancer, inflammatory disorders, respiratory disorders, bacterial infections, and fungal infections. Use of deuterium-enriched compounds having high enantiomeric purity is contemplated to maximize therapeutic benefits, such as achieving increased potency per dose of therapeutic agent and minimize adverse side effects.

Accordingly, one aspect of the invention provides a method of treating a medical disorder in a patient. The method comprises administering to a patient in need thereof a therapeutically effective amount of a compound described herein, such as a deuterium-enriched compound described in Section 1 above, to treat the disorder. In certain embodiments, the deuterium-enriched compound is a compound of Formula I-A, I-B, I-C, I-D, I-E, I-F, I-G, I-H, or I-I. In certain embodiments, the deuterium-enriched compound is a compound of Formula I, I-A, I-B, I-C, I-D, I-E, I-F, I-G, I-H, or I-I. In certain embodiments, the compound is administered orally. Exemplary medical disorders for treatment are described in more detail below.

Another aspect of the invention provides a method of treating a medical disorder in a patient. The method comprises orally administering to a patient in need thereof a therapeutically effective amount of a compound of Formula I-J to treat the disorder. Exemplary medical disorders for treatment are described in more detail below.

Another aspect of the invention provides a method of inducing death of a bacterial cell. The method comprises exposing a bacterial cell to an effective amount of a deuterium-enriched compound described herein to induce death of said bacterial cell. In certain embodiments, the method comprises inducing death of a population of bacterial cells.

Another aspect of the invention provides a method of inducing death of a fungus. The method comprises exposing a fungus to an effective amount of a deuterium-enriched compound described herein to induce death of said fungus. In certain embodiments, the method comprises inducing death of a population of fungi.

### Metabolic Disorders

In certain embodiments, the disorder is a metabolic disorder. Exemplary metabolic disorders include, for example, diabetes (e.g., type I diabetes and type II diabetes), nonalcoholic steatohepatitis, non-alcoholic fatty liver disease, viral hepatitis, liver cirrhosis, liver fibrosis, diabetic retinopathy, diabetic neuropathy, diabetic nephropathy, beta cell depletion, insulin resistance in a patient with congenital adrenal hyperplasia treated with a glucocorticoid, dysmetabolism in peritoneal dialysis patients, reduced insulin secretion, improper distribution of brown fat cells and white fat cells, obesity, and improper modulation of leptin levels. In certain embodiments, the metabolic disorder is further selected from a complication of diabetes. In certain embodiments, the metabolic disorder is type I diabetes, non-alcoholic fatty liver disease, viral hepatitis, liver cirrhosis, liver fibrosis, diabetic retinopathy, diabetic neuropathy, diabetic nephropathy, beta cell depletion, insulin resistance in a patient with congenital adrenal hyperplasia treated with a glucocorticoid, dysmetabolism in peritoneal dialysis patients, reduced insulin secretion, improper distribution of brown fat cells and white fat cells, obesity, or improper rnodulation of leptin levels. In certain embodiments, the metabolic disorder is non-alcoholic fatty liver disease, diabetic retinopathy, diabetic neuropathy, diabetic nephropathy, beta cell depletion, or insulin resistance in a patient with congenital adrenal hyperplasia treated with a glucocorticoid. In certain embodiments, the metabolic disorder is non-alcoholic fatty liver disease, diabetic retinopathy, diabetic neuropathy, diabetic nephropathy, beta cell depletion, reduced insulin secretion, improper distribution of brown fat cells and white fat cells, obesity, or improper modulation of leptin levels. In certain other embodiments, the metabolic disorder is non-alcoholic fatty liver disease. In certain other embodiments, the metabolic disorder is beta cell loss treatable by beta-cell regeneration. In certain other embodiments, the metabolic disorder is central obesity, dyslipidemia, or pre-diabetes.

In certain embodiments, the disorder is diabetes (e.g., type I diabetes and type II) diabetes. In certain embodiments, the disorder is type II diabetes. In certain embodiments, the disorder is nonalcoholic steatohepatitis.

### Neurological Disorders

In certain embodiments, the disorder is a neurological disorder. Exemplary neurological disorders include, for example, Alzheimer's disease, Parkinson's disease, amyotrophic lateral sclerosis, Friedreich's ataxia, autism spectrum disorder, depression, mild cognitive impairment, Down syndrome, neurodegeneration, adrenoleukodystrophy, Huntington's disease, stroke, traumatic brain injury, substance abuse, spinal cord injury, neuronal injury, major depression or bipolar disorder comorbid with metabolic syndrome, and a neurological disorder caused by functional mitochondrial impairment. In certain embodiments, the neurological disorder is selected from the group consisting of Alzheimer's disease, Parkinson's disease, amyotrophic lateral sclerosis, Friedreich's ataxia, autism spectrum disorder, depression, mild cognitive impairment, neurodegeneration, adrenoleukodystrophy, Huntington's disease, stroke, traumatic brain injury, substance abuse, spinal cord injury, neuronal injury, and major depression or bipolar disorder comorbid with metabolic syndrome. In certain embodiments, the neurological disorder is selected from the group consisting of Alzheimer's disease, Parkinson's disease, amyotrophic lateral sclerosis, Friedreich's ataxia, depression, mild cognitive impairment, neurodegeneration, adrenoleukodystrophy, and Huntington's disease. In certain other embodiments, the neurological disorder is Alzheimer's disease. In certain other embodiments, the neurological disorder is Down syndrome.

In certain other embodiments, the neurological disorder is a cognitive disorder, such as cognitive impairment and/or memory impairment. The cognitive impairment may be, for example, cognitive impairment associated with Alzheimer's disease.

In certain embodiments, the disorder is substance abuse, such as alcohol craving, heroin dependence, and/or nicotine dependence.

### Cancer

In certain embodiments, the disorder is cancer. Exemplary cancers include, for example, lung cancer, hepatocellular carcinoma, astrocytoma, glioma, glioblastoma, meningioma, liver cancer, lymphoma, melanoma, multiple myeloma, pancreatic cancer, colorectal cancer, pituitary cancer, thyroid cancer, esophageal cancer, and prostate cancer. In certain embodiments, the cancer is non-small cell lung cancer or hepatocellular carcinoma.

In certain other embodiments, the cancer is lung cancer, hepatocellular carcinoma, astrocytoma, glioma, glioblastoma, meningioma, liver cancer, lymphoma, melanoma, multiple myeloma, pancreatic cancer, colorectal cancer, pituitary cancer, thyroid cancer, esophageal cancer, prostate cancer, ear cancer, nose cancer, throat cancer, kidney cancer, breast cancer, stomach cancer, or uterine cancer. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratosis, acute lymphocytic leukemia, acute myeloid leukemia, adenoid cystic carcinoma, adenoma, adenosarcoma, adenosquamous carcinoma, anal canal cancer, anal cancer, anorectal cancer, astrocytic tumor, Bartholin's gland carcinoma, basal cell carcinoma, biliary cancer, bone cancer, bone marrow cancer, bronchial cancer, bronchial gland carcinoma, carcinoid, cholangiocarcinoma, chorioid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastoma, hemangioendothelioma, hemangioma, hepatic adenoma, hepatic adenomatosis, hepatobiliary cancer, hepatocellular carcinoma, Hodgkin's disease, ileum cancer, insulinoma, intraepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanoma, lymphoma, male genital cancer, malignant melanoma, malignant mesothelial tumor, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma, nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumor, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T-cell leukemia, tongue cancer, undifferentiated carcinoma, ureter cancer, urethral cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In certain other embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

### Respiratory Disorders

In certain embodiments, the disorder is a respiratory disorder. Exemplary respiratory disorders include, for example, chronic obstructive pulmonary disease, asthrma, bronchitis, cystic fibrosis, pulmonary edema, pulmonary embolism, pulmonary arterial hypertension, pneumonia, pulmonary sarcoidosis, silicosis, pulmonary fibrosis, respiratory failure, acute respiratory distress syndrome, emphysema, chronic bronchitis, tuberculosis, lung cancer, and a chronic respiratory condition. In certain embodiments, the respiratory disorder is chronic obstructive pulmonary disease, asthma, or a chronic respiratory condition. In certain other embodiments, the respiratory disorder is chronic obstructive pulmonary disease. In yet other embodiments, the respiratory disorder is bronchitis, cystic fibrosis, pulmonary edema, pulmonary embolism, pneumonia, pulmonary sarcoidosis, silicosis, pulmonary fibrosis, respiratory failure, acute respiratory distress syndrome, emphysema, chronic bronchitis, tuberculosis, or lung cancer. In certain embodiments, the asthma is mild asthma, moderate asthma, severe asthma, or steroid-resistant asthma.

### Symptom of Hepatitis

In certain embodiments, the disorder is a symptom of hepatitis.

### Cardiovascular Disease

In certain embodiments, the disorder is a cardiovascular disease. Exemplary cardiovascular diseases include, for example, hypertension, hyperlipidemia, atherosclerosis, improper vascular function, dyslipidemia, stenosis, restenosis, myocardial infarction, stroke, intracranial hemorrhage, acute coronary syndrome, stable angina pectoris, and unstable angina pectoris. In certain other embodiments, the cardiovascular disorder is intracranial hemorrhage, acute coronary syndrome, stable angina pectoris, or unstable angina pectoris.

In another aspect, the invention provides a method for preventing stroke in a patient. The method comprises administering to a patient in need thereof a therapeutically effective amount of a deuterium-enriched compound described herein to prevent said stroke.

The method of treatment or the method of prevention may involve a patient at risk for central nervous system ischemic stroke, or may involve a patient at risk for stroke due to cardiovascular disease,

### Inflammatory or Immune-Mediated Disorder

In certain embodiments, the medical disorder is an inflammatory or immune-mediated disorder. Exemplary inflammatory or immune-mediated disorders include, for example, chronic kidney disease, arthritis, a primary cicatricial alopecia, lung fibrosis, multiple sclerosis, endotoxemia, sepsis, septic shock, laminitis, inflammatory bowel disease, colitis, Crohn's disease, rheumatoid arthritis, lupus, myasthenia gravis, vasculitis, chronic pancreatitis, a hyperproliferative skin disorder, an inflammatory skin disorder, rhinitis (e.g., allergic rhinitis), and a dermatological condition, In certain embodiments, the inflammatory or immune-mediated disorder is selected from the group consisting of chronic kidney disease, arthritis, a primary cicatricial alopecia, lung fibrosis, multiple sclerosis, endotoxemia, sepsis, septic shock, laminitis, inflamratory bowel disease, colitis, Crohn's disease, rheumatoid arthritis, lupus, myasthenia gravis, vasculitis, chronic pancreatitis, a hyperproliferative skin disorder, an inflammatory skin disorder, and a dermatological condition. In certain embodiments, the chronic kidney disease may be, for example, polycystic kidney disease (such as autosomal dominant or autosomal recessive).

### Dermatological Disorder

In certain embodiments, the disorder is a dermatological disorder, such as psoriasis, atopic dermatitis, acne, leukoplakia, scleroderma, or a skin malignancy.

### Additional Medical Disorders

Additional medical disorders contemplated for treatment include transplant rejection, liver functional impairment, Rabson-Mendenhall syndrome, Donohue syndrome, Leber hereditary optic neuropathy, myotonic dystrophy, ototoxicity, Niemann Pick disease, autosomal dominant optic atrophy, spinal bulbar muscular atrophy, Mohr-Tranebjaerg syndrome, hereditary spastic paraplegia, MELAS syndrome, monoclonal immunoglobulin deposition disease (MIDD), deafness, insulin resistance in a patient receiving growth hormone, and chronic progressive external ophthalmoplegia with mitochondrial myopathy.

### Bacterial Infections

In certain embodiments, the disorder to be treated is a bacterial infection. Bacteria can be characterized according to classifications known in the art. For example, in certain embodiments, the bacteria is a gram-positive bacteria, such as a gram-positive coccus bacteria or a gram-positive bacillus bacteria. In other embodiments, the bacteria is a gram-negative bacteria, such as a gram-negative coccus bacteria or a gram-negative bacillus bacteria. The bacteria can also be characterized according to whether it an anaerobic or aerobic bacteria. Accordingly, in certain embodiments, the bacteria is an anaerobic bacteria. In certain other embodiments, the bacteria is an aerobic bacteria.

A variety of bacteria are contemplated to be susceptible to the deuterium-enriched compounds herein. Representative bacteria include Staphylococcus species, e.g., S. aureus; Enterococcus species, e.g., E. faecalis and E. faecium; Streptococcus species, e.g., S. pyogenes and S. pneumoniae; Escherichia species, e.g., E. coli, including enterotoxigenic, enteropathogenic, enteroinvasive, enterohemorrhagic and enteroaggregative E. coli strains; Haemophilus species, e.g., H. influenza; and Moraxella species, e.g., M. catarrhalis. Other examples include Mycobacteria species, e.g., M. tuberculosis, M. avian-intracellulare, M. kansasii, M. bovis, M. africanum, M. genavense, M. leprae, M. xenopi, M. simiae, M. scrofulaceum, M. malmoense, M. celatum, M. abscessus, M. chelonae, M. szulgai, M. gordonae, M. haemophilum, M. fortuni and M. marinum; Corynebacteria species, e.g., C. diphtheriae; Vibrio species, e.g., V. cholerae; Campylobacter species, e.g., C. jejuni; Helicobacter species, e.g., H. pylori; Pseudomonas species, e.g., P. aeruginosa; Legionella species, e.g., L. pneumophila; Treponema species, e.g., T. pallidum; Borrelia species, e.g., B. burgdorferi; Listeria species, e.g., L. monocytogenes; Bacillus species, e.g., B. cereus; Bordatella species, e.g., B. pertussis; Clostridium species, e.g., C. perfringens, C. tetani, C. difficile and C. botulinum; Neisseria species, e.g., N. meningitidis and N. gonorrhoeae; Chlamydia species, e.g., C. psittaci, C. pneumoniae and C. trachomatis; Rickettsia species, e.g., R. rickettsii and R. prowazekii; Shigella species, e.g., S. sonnei; Salmonella species, e.g., S. typhimurium; Yersinia species, e.g., Y. enterocolitica and Y. pseudotuberculosis; Klebsiella species, e.g., K. pneumoniae; Mycoplasma species, e.g., M. pneumoniae; and Trypanosoma brucei. In certain embodiments, the compounds described herein are used to treat a patient suffering from a bacterial infection selected from the group consisting of S. aureus, E. faecalis, E. faecium, S. pyogenes, S. pneumonia, and P. aeruginosa.

In yet other embodiments, the bacteria is a member of the genus Peptostreptococcus, a Peptostreptococcus asaccharolyticus, a Peptostreptococcus magnus, a Peptostreptococcus micros, a Peptostreptococcus prevotii, a member of the genus Porphyromonas, a Porphyromonas asaccharolytica, a Porphyromonas canoris, a Porphyromonas gingivalis, a Porphyromonas macaccae, a member of the genus Actinomyces, an Actinomyces israelii, an Actinomyces odontolyticus, a member of the genus Clostridium, a Clostridium innocuum, a Clostridium clostridioforme, a Clostridium difficile, a member of the genus Anaerobiospirillum, a member of the genus Bacteroides, a Bacteroides tectum, a Bacteroides ureolyticus, a Bacteroides gracilis (Campylobacter gracilis), a member of the genus Prevotella, a Prevotella intermedia, a Prevotella heparinolytica, a Prevotella oris-buccae, a Prevotella bivia, a Prevotella melaninogenica, a member of the genus Fusobacterium, a Fusobacterium naviforme, a Fusobacterium necrophorum, a Fusobacterium varium, a Fusobacterium ulcerans, a Fusobacterium russii, a member of the genus Bilophila, or a Bilophila wadsworthia.

In yet other embodiments, methods herein involve treatment of an infection by one or more of a Streptococccus, Escherichia, Klebsiella, Acinetobacter, Actinomyces, Anaerobiospirillum, Bacillus, Bacteroides, Bilophila, Campylobacter, Clostridium, Enterococcus, Eubacterium, Francisella, Fusobacterium, Haemophilus, Listeria, Moraxella, Mycobacterium, Neisseria, Peptostreptococcus, Porphyromonas, Prevotella, Proteus, Pseudomonas, Salmonella, or Yersinia. In certain embodiments, the bacterial infection is an infection by one or more Streptococccus species, Escherichia species, Klebsiella species, Actinomyces species, Enterococcus species, Mycobacterium species, Neisseria species, or Pseudomonas species. In certain other embodiments, the bacterial infection is an infection by one or more of Streptococcus pneumoniae, Streptococcus pyogenes, Staphylococcus aureus, Staphylococcus epidermidis, Acinetobacter baumannii, Bacillus anthracis, Bacteroides fragilis, Clostridium perfringens, Clostridium difficile, Escherichia coli, Enterococcus faecalis, Enterococcus faecium, Eubacterium lentum, Francisella tularensis, Fusobacterium nucleatum, Haemophilus influenzae, Klebsiella pneumoniae, Moraxella catarrhalis, Mycobacterium smegmatis, Mycobacterium tuberculosis, Neisseria gonorrhoeae, Porphyromonas asaccharolyticus, Proteus mirabilis, Pseudomonas aeruginosa, Salmonella typhimurium, or Yersinia enterocolytica. In certain other embodiments, the bacterial infection is an infection by Streptococccus pneumoniae, Escherichia coli, or Klebsiella pneumoniae.

The antibacterial activity of compounds described herein may be evaluated using assays known in the art, such as the microbroth dilution minimum inhibition concentration (MIC) assay, as further described in National Committee for Clinical Laboratory Standards. Performance Standards for Antimicrobial Susceptibility Testing; Fourteenth Informational Supplement. NCCLS document M100-S14 {ISBN 1-56238-516-X}. This assay may be used to determine the minimum concentration of a compound necessary to prevent visible bacterial growth in a solution. In general, the drug to be tested is serially diluted into wells, and aliquots of liquid bacterial culture are added. This mixture is incubated under appropriate conditions, and then tested for growth of the bacteria. Compounds with low or no antibiotic activity (a high MIC) will allow growth at high concentrations of compound, while compounds with high antibiotic activity will allow bacterial growth only at lower concentrations (a low MIC).

The assay uses stock bacterial culture conditions appropriate for the chosen strain of bacteria. Stock cultures from the permanent stock culture collection can be stored as frozen suspensions at −70° C. Cultures may be suspended in 10% skim milk (BD) prior to snap freezing in dry ice/ethanol and then placed in a −70° C. freezer. Cultures may be maintained on Tryptic Soy Agar containing 5% Sheep Blood at room temperature (20° C.), and each culture may be recovered from frozen form and transferred an additional time before MIC testing. Fresh plates are inoculated the day before testing, incubated overnight, and checked to confirm purity and identity.

The identity and purity of the cultures recovered from the stock culture can be confirmed to rule out the possibility of contamination. The identity of the strains may be confirmed by standard microbiological methods (See, e.g., Murray et al., Manual of Clinical Microbiology, Eighth Edition. ASM Press {ISBN 1-55581-255-4}). In general, cultures are streaked onto appropriate agar plates for visualization of purity, expected colony morphology, and hemolytic patterns. Gram stains can also be utilized. The identities are confirmed using a MicroScan WalkAway 40 SI Instrument (Dade Behring, West Sacramento, Calif.). This device utilizes an automated incubator, reader, and computer to assess for identification purposes the biochemical reactions carried out by each organism. The MicroScan WalkAway can also be used to determine a preliminary MIC, which may be confirmed using the method described below.

Frozen stock cultures may be used as the initial source of organisms for performing microbroth dilution minimum inhibition concentration (MIC) testing. Stock cultures are passed on their standard growth medium for at least 1 growth cycle (18-24 hours) prior to their use. Most bacteria may be prepared directly from agar plates in 10 mL aliquots of the appropriate broth medium. Bacterial cultures are adjusted to the opacity of a 0.5 McFarland Standard (optical density value of 0.28-0.33 on a Perkin-Elmer Lambda EZ150 Spectrophotometer, Wellesley, Mass., set at a wavelength of 600 nm). The adjusted cultures are then diluted 400 fold (0.25 mL inoculum+100 mL broth) in growth media to produce a starting suspension of approximately 5×105 colony forming units (CFU)/mL. Most bacterial strains may be tested in cation adjusted Mueller Hinton Broth (CAMHB).

Test compounds (“drugs”) are solubilized in a solvent suitable for the assay, such as DMSO. Drug stock solutions may be prepared on the day of testing. Microbroth dilution stock plates may be prepared in two dilution series, 64 to 0.06 μg drug/mL and 0.25 to 0.00025 μg drug/mL. For the high concentration series, 200 μL of stock solution (2 mg/mL) is added to duplicate rows of a 96-well microtiter plate. This is used as the first well in the dilution series. Serial two-fold decremental dilutions are made using a BioMek FX robot (Beckman Coulter Inc., Fullerton, Calif.) with 10 of the remaining 11 wells, each of which will contain 100 μL of the appropriate solvent/diluent. Row 12 contains solvent/diluent only and serves as the control. For the first well of the low concentration series, 200 μL of an 8 μg/mL stock are added to duplicate rows of a 96-well plate. Serial two-fold dilutions are made as described above.

Daughter 96-well plates may be spotted (3.2 μL/well) from the stock plates listed above using the BioMek FX robot and used immediately or frozen at −70° C. until use. Aerobic organisms are inoculated (100 μL volumes) into the thawed plates using the BioMek FX robot. The inoculated plates are be placed in stacks and covered with an empty plate. These plates are then incubated for 16 to 24 hours in ambient atmosphere according to CLSI guidelines (National Committee for Clinical Laboratory Standards, Methods for Dilution, Antimicrobial Tests for Bacteria that Grow Aerobically; Approved Standard-Sixth Edition. NCCLS document M7-A6 {ISBN 1-56238-486-4}).

After inoculation and incubation, the degree of bacterial growth can be estimated visually with the aid of a Test Reading Mirror (Dynex Technologies 220 16) in a darkened room with a single light shining directly through the top of the microbroth tray. The MIC is the lowest concentration of drug that prevents macroscopically visible growth under the conditions of the test.

### Fungal Infections

In certain embodiments, the disorder to be treated is a fungal infection. Exemplary fungi that may be treated include, for example, a fungus from the genus Acremonium, Absidia, Alternaria, Aspergillus, Aureobasidium, Basidiobolus, Bjerkandera, Blastomyces, Candida, Cephalosporium, Ceriporiopsis, Chaetomium, Chrysosporium, Cladosporium, Coccidioides, Conidiobolus, Coprinus, Coriolus, Corynespora, Cryptococcus, Curvularia, Cunninghamella, Exophiala, Epidermophyton, Filibasidium, Fonsecaea, Fusarium, Geotrichum, Hendersonula, Histoplasma, Humicola, Leptosphaeria, Loboa, Madurella, Malassezia, Microsporum, Mycocentrospora, Mucor, Neotestudina, Paecilomyces, Paracoccidioides, Penicillium, Phialophora, Pneumocystis, Pseudallescheria, Rhinosporidium, Rhizomucor, Rhizopus, Saccharomyces, Scopulariopsis, Sporothrix, Talaromyces, Thermoascus, Thielavia, Tolypocladium, Trametes, Trichoderma, Trichophyton, Trichosporon, or Wangiella. In certain embodiments, the fungus is an Acremonium, Absidia (e.g., Absidia corymbifera), Alternaria, Aspergillus (e.g., Aspergillus clavatus, Aspergillus flavus, Aspergillus fumigatus, Aspergillus nidulans, Aspergillus niger, Aspergillus terreus, and Aspergillus versicolor), Aureobasidium, Basidiobolus, Blastomyces (e.g., Blastomyces dermatitidis), Candida (e.g., Candida albicans, Candida glabrata, Candida guilliermondii, Candida kefyr, Candida krusei, Candida lusitaniae, Candida parapsilosis, Candida pseudotropicalis, Candida stellatoidea, Candida tropicalis, Candida utilis, Candida lipolytica, Candida famata and Candida rugosa), Cephalosporium, Chaetomium, Chrysosporium, Cladosporium (e.g., Cladosporium carrionii and Cladosporium trichloides), Coccidioides (e.g., Coccidioides immitis), Conidiobolus, Coprinus, Corynespora, Cryptococcus (e.g., Cryptococcus neoformans), Curvularia, Cunninghamella (e.g., Cunninghamella elegans), Exophiala (e.g., Exophiala dermatitidis and Exophiala spinfera), Epidermophyton (e.g., Epidermophyton floccosum), Fonsecaea (e.g., Fonsecaea pedrosoi), Fusarium (e.g., Fusarium solani), Geotrichum (e.g., Geotrichum candiddum and Geotrichum clavatum), Hendersonula, Histoplasma, Leptosphaeria, Loboa, Madurella, Malassezia (e.g., Malassezia furfur), Microsporum (e.g., Microsporum canis and Microsporum gypseum), Mycocentrospora, Mucor, Neotestudina, Paecilomyces, Paracoccidioides (e.g., Paracoccidioides brasiliensis), Penicillium (e.g., Penicillium marneffei), Phialophora, Pneumocystis (e.g., Pneumocystis carinii), Pseudallescheria (e.g., Pseudallescheria boydii), Rhinosporidium, Rhizomucor, Rhizopus (e.g., Rhizopus microsporus var. rhizopodiformis and Rhizopus oryzae), Saccharomyces (e.g., Saccharomyces cerevisiae), Scopulariopsis, Sporothrix (e.g., Sporothrix schenckii), Trichophyton (e.g., Trichophyton mentagrophytes and Trichophyton rubrum), Trichosporon (e.g., Trichosporon asahii, Trichosporon beige/ii and Trichosporon cutaneum), and Wangiella.

In certain other embodiments, the fungus is Aspergillus awamori, Aspergillus foetidus, Aspergillus funiigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearurn, Fusarium graminum, Fusarium heterosporum, Fusarium negimdi, Fusarium oxvsporum, Fusarium reticulatum, Fusarium roseum, Fusarium sambucinum, Fusarium sarcochrourn, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpiirogenum, Phanerochaete chrysosporium, Phlehia radiata, Pleurolus eryngii, Thielavia terrestris, Trametes villosa, Trametes versicolor, Trichoderma harzianum, Trichoderma koningii, Trichoderma longibrachiatiim, Trichoderma reesei, or Trichoderma viride. In certain embodiments, methods herein comprise treating an infection by one or more of Aspergillus awamori, Aspergillus foetidus, Aspergillus funiigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearurn, Fusarium graminum, Fusarium heterosporum, Fusarium negimdi, Fusarium oxvsporum, Fusarium reticulatum, Fusarium roseum, Fusarium sambucinum, Fusarium sarcochrourn, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpiirogenum, Phanerochaete chrysosporium, Phlehia radiata, Pleurolus eryngii, Thielavia terrestris, Trametes villosa, Trametes versicolor, Trichoderma harzianum, Trichoderma koningii, Trichoderma longibrachiatiim, Trichoderma reesei, or Trichoderma viride.

### Reducing the Amount of a Triglyceride or Low-Density Lipoprotein

Another aspect of the invention provides a method of reducing the amount of a triglyceride or low-density lipoprotein (LDL) in a patient. The method comprises administering to a patient in need thereof an effective amount of a deuterium-enriched compound described herein to reduce the amount of a triglyceride or LDL in the patient. In certain embodiments, the method provides a reduction of at least 1%, 5%, 10%, or 25% in the amount of a triglyceride or low-density lipoprotein (LDL) in the patient.

### Increasing the Amount of High-Density Lipoprotein

Another aspect of the invention provides a method of increasing the amount of high-density lipoprotein (HDL) in a patient. The method comprises administering to a patient in need thereof a therapeutically effective amount of a deuterium-enriched compound described herein to increase the amount of HDL in the patient. In certain embodiments, the method provides an increase of at least 1%, 5%, 10%, or 25% in the amount of high-density lipoprotein (HDL) in a patient.

### Modulating Expression of Cytokines

Another aspect of the invention provides a method of modulating expression of a pro-inflammatory cytokine (e.g., TNFα, IL-1β, or IL-6) in a patient suffering from an inflammatory disorder. The method comprises administering to a patient in need thereof an effective amount of a deuterium-enriched compound described herein to modulate expression of the pro-inflammatory cytokine. In certain embodiments, the pro-inflammatory cytokine is TNFα.

Another aspect of the invention provides a method of modulating expression of an anti-inflammatory cytokine in a patient suffering from an inflammatory disorder. The method comprises administering to a patient in need thereof an effective amount of a deuterium-enriched compound described herein to modulate expression of the anti-inflammatory cytokine.

### Modulating Macrophage Function

Another aspect of the invention provides a method of modulating macrophage function in a patient suffering from a disease. The method comprises administering to a patient in need thereof an effective amount of a deuterium-enriched compound described herein to modulate macrophage function.

### Method of Promoting Wound Healing

Another aspect of the invention provides a method of promoting wound healing. The method comprises administering to a patient in need thereof a therapeutically effective amount of a deuterium-enriched compound described herein to promote wound healing.

### Treating Skin Defects

Another aspect of the invention provides a method of treating skin defects caused by exposure to ultraviolet radiation. The method comprises administering to a patient in need thereof a therapeutically effective amount of a deuterium-enriched compound described herein to treat skin defects caused by exposure to ultraviolet radiation.

### Method of Modulating Stem Cell Differentiation

Another aspect of the invention provides a method of modulating stem cell differentiation, such as in a patient. The method comprises exposing a stem cell to a deuterium-enriched compound described herein to modulate stem cell differentiation.

### Preventing Medical Disorders

Also provided are methods of preventing a medical disorder in a patient. The method comprises administering to a patient in need thereof an effective amount of a deuterium-enriched compound described herein to prevent the medical disorder. The medical disorder may be one or more of the medical disorders recited above, such as a neurological disorder (e.g., Alzheimer's disease or Parkinson's disease), cancer (e.g., non-small cell lung cancer or hepatocellular carcinoma), a metabolic disorder, a cardiovascular disorder (e.g. in-stent renarrowing in diabetes patients, reinfarction in diabetes patients, or cardiac allograft vasculopathy after heart transplant), or a respiratory disorder (e.g., chronic obstructive pulmonary disease).

### Additional Medical Uses

Also provided are methods of using compounds herein for therapy comprising regenerative medicine. Also provided are methods of treating veterinary disorders, such as laminitis, using a compound described herein, such as a compound of Formula I, II, III, IV, or IV, having a stereochemical purity of at least 75% enantiomeric excess to treat the veterinary disorder.

### Manufacture of Medicaments

Another aspect of the invention provides for the use of a deuterium-enriched compound described herein in the manufacture of a medicament. The medicament may be for treating one or more of the medical disorders described herein, such as treating a neurological disorder (e.g., Alzheimer's disease or Parkinson's disease), cancer (e.g., non-small cell lung cancer or hepatocellular carcinoma), a metabolic disorder, or a respiratory disorder (e.g., chronic obstructive pulmonary disease).

### III. Dosing Considerations and Combination Therapy

Doses of a compound provided herein, or a pharmaceutically acceptable salt thereof, vary depending on factors such as: specific indication to be treated; age and condition of a patient; and amount of second active agent used, if any. Generally, a compound provided herein, or a pharmaceutically acceptable salt thereof, may be used in an amount of from about 0.1 mg to about 1 g per day, or from about 0.1 mg to about 500 mg per day, and can be adjusted in a conventional fashion (e.g., the same amount administered each day of the treatment), in cycles (e.g., one week on, one week off), or in an amount that increases or decreases over the course of treatment. In other embodiments, the dose can be from about 1 mg to about 500 mg, from about 0.1 mg to about 150 mg, from about 1 mg to about 300 mg, from about 10 mg to about 100 mg, from about 0.1 mg to about 50 mg, from about 1 mg to about 50 mg, from about 10 mg to about 50 mg, from about 20 mg to about 30 mg, or from about 1 mg to about 20 mg.

In yet other embodiments, the daily dose can be from about 1 mg to 5 mg, 5 mg to 10 mg, 10 mg to 20 mg, 20 mg to 35 mg, 35 mg to 50 mg, 50 mg to 75 mg, 75 mg to 100 mg, 100 mg to 125 mg, 125 mg to 150 mg, 150 mg to 175 mg, 175 mg to 200 mg, 200 mg to 225 mg, 225 mg to 250 mg, 250 mg to 275 mg, 275 mg to 300 mg, 300 mg to 325 mg, 325 mg to 350 mg, 350 mg to 375 mg, 375 mg to 400 mg, 400 mg to 425 mg, 425 mg to 450 mg, 450 mg to 475 mg, or 475 mg to 500 mg. In certain embodiments, the daily dosage is in the range of about 1 mg to 50 mg, 50 mg to 100 mg, 100 mg to 150 mg, 150 mg to 200 mg, 200 mg to 250 mg, 250 mg to 300 mg, 300 mg to 350 mg, 350 mg to 400 mg, or 400 mg to 500 mg. In yet other embodiments, the daily dose is less than about 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, or 450 mg. In yet other embodiments, the daily dose is less than about 125 mg, 150 mg, or 175 mg.

Unless indicated otherwise, compounds described herein may be administered using any medically accepted route of administration. For example, in certain embodiments, unless indicated otherwise, the compound is administered by oral administration, injection, or transdermal administration. In a preferred embodiment, the compound is administered orally.

In certain aspects, the therapeutic agents provided herein are cyclically administered to a patient. Cycling therapy involves the administration of an active agent for a period of time, followed by a rest (i.e., discontinuation of the administration) for a period of time, and repeating this sequential administration. Cycling therapy can reduce the development of resistance to one or more of the therapies. These regimens can avoid or reduce the side effects of one of the therapies, and/or improve the efficacy of the treatment.

Consequently, in another aspect, a compound provided herein is administered daily in a single or divided doses in a four to six week cycle with a rest period of about a week or two weeks. Cycling therapy further allows the frequency, number, and length of dosing cycles to be increased. Thus, another aspect encompasses the administration of a compound provided herein for more cycles than are typical when it is administered alone. In yet another aspect, a compound provided herein is administered for a greater number of cycles than would typically cause dose-limiting toxicity in a patient to whom a second active ingredient is not also being administered.

In another aspect, a compound provided herein is administered daily and continuously for three or four weeks at a dose of from about 0.1 mg to about 500 mg per day, followed by a rest of one or two weeks. In other embodiments, the dose can be from about 1 mg to about 500 mg, from about 0.1 mg to about 150 mg, from about 1 mg to about 300 mg, from about 10 mg to about 100 mg, from about 0.1 mg to about 50 mg, from about 1 mg to about 50 mg, from about 10 mg to about 50 mg, from about 20 mg to about 30 mg, or from about 1 mg to about 20 mg, followed by a rest.

In another aspect, a compound provided herein and a second active ingredient are administered orally or parenterally, with administration of the compound provided herein occurring prior to (e.g., about 30 to 60 minutes) the second active ingredient, during a cycle of four to six weeks. In certain embodiments, the compound and second active agent are administered as a single dosage or they are administered separately. In another aspect, the combination of a compound provided herein and a second active ingredient is administered by intravenous infusion over about 90 minutes every cycle.

Typically, the number of cycles during which the combination treatment is administered to a patient will be from about one to about 24 cycles, from about two to about 16 cycles, or from about three to about four cycles.

### Combination Therapy

A compound provided herein, or a pharmaceutically acceptable salt thereof, can be combined with other pharmacologically active compounds (“second active agents”) in methods and compositions provided herein. Certain combinations may work synergistically in the treatment of particular types of diseases or disorders, and conditions and symptoms associated with such diseases or disorders. A compound provided herein, or a pharmaceutically acceptable salt thereof, can also work to alleviate adverse effects associated with certain second active agents, and vice versa.

One or more second active ingredients or agents can be used in the methods and compositions provided herein. Second active agents can be large molecules (e.g., proteins) or small molecules (e.g., synthetic inorganic, organometallic, or organic molecules).

In certain embodiments, the combination therapy comprises a deuterium-enriched compound described herein and a second therapeutic agent for treating a metabolic disorder, such as metformin, a dipeptidyl peptidase IV inhibitor (e.g., sitagliptin, vildagliptin, or the like), a statin (e.g., a HMG-CoA reductase inhibitor, such as atorvastatin, cerivastatin, fluvastatin, lovastatin, mevastatin, simvastatin, rosuvastatin, pravastatin, or combination thereof), a GLP-1 agonist, a GLP-2 agonist, or an SGLT2 inhibitor. As appreciated, the combination therapy may comprising more than two therapeutic agents, such as where a combination of a deuterium-enriched compound described herein and at least two of the aforementioned agents for treating a metabolic disorder are administered to the patient.

In certain other embodiments, the combination therapy comprises a deuterium-enriched compound described herein and a diuretic agent, such as hydrochlorothiazide.

In certain other embodiments, the combination therapy comprises a deuterium-enriched compound described herein and a second therapeutic agent for treating hypertension, diabetes, or an inflammatory disorder. The second therapeutic agent may be one that limits the activity of the renin-angiotensin system, such as an angiotensin converting enzyme inhibitor (e.g., an ACE inhibitor, such as ramipril, captopril, enalapril, or the like), an angiotensin receptor blocker (e.g., candesartan, losartan, olmesartan, or the like), or a renin inhibitor. Alternatively, the second therapeutic agent may limit hypertension by alternate means, such as a beta-adrenergic receptor blocker or calcium channel blocker (e.g., amlodipine).

In certain other embodiments, the combination therapy comprises a deuterium-enriched compound described herein and a glucocorticoid agonist. Such combination therapy may be particularly useful for treating an inflammatory disorder, such as therapy for suppressing an immune response, preventing transplant rejection, and treating autoimmune disease. Exemplary disorders include, for example, rheumatoid arthritis, lupus, myasthenia gravis, muscular dystrophy vasculitis, multiple sclerosis, chronic obstructive pulmonary disease (COPD), inflammatory bowel disease, treatment of acute allergic reactions, and transplant rejection. In certain other embodiments, the combination therapy comprises a deuterium-enriched compound described herein and a second therapeutic agent for treating a kidney disease. Exemplary such second therapeutic agents include those that increase cAMP or comprise a beta-adrenergic agonist. Exemplary beta-adrenergic agonists include, for example, a beta-1-adrenergic agonist, a beta-2-adrenergic agonist, a beta-3-adrenergic agonist, or a combination thereof. In certain embodiments, the second therapeutic agent is noradrenaline, isoprenaline, dobutamine, salbutamol, levosalbutamol, terbutaline, pirbuterol, procaterol, metaproterenol, fenoterol, bitolterol mesylate, salmeterol, formoterol, bambuterol, clenbuterol, indacaterol, L-796568, amibegron, solabegron, isoproterenol, albuterol, metaproterenol, arbutamine, befunolol, bromoacetylalprenololmenthane, broxaterol, cimaterol, cirazoline, denopamine, dopexamine, epinephrine, etilefrine, hexoprenaline, higenamine, isoetharine, isoxsuprine, mabuterol, methoxyphenamine, nylidrin, oxyfedrine, prenalterol, ractopamine, reproterol, rimiterol, ritodrine, tretoquinol, tulobuterol, xamoterol, zilpaterol, zinterol, or a pharmaceutically acceptable salt thereof; or a combination of any of the foregoing.

In certain other embodiments, the combination therapy comprises a deuterium-enriched compound described herein and a second therapeutic agent for treating an inflammatory disease. The second therapeutic agent for treating an inflammatory disease may be, for example, a non-steroidal anti-inflammatory drug, such as salicylic acid, aspirin, methyl salicylate, diflunisal, salsalate, olsalazine, sulfasalazine, indomethacin, sulindac, etodolac, tolmetin, ketorolac, diclofenac, ibuprofen, naproxen, fenoprofen, ketoprofen, flurbiprofen, oxaprozin, piroxicam, celecoxib, rofecoxib, or a pharmaceutically acceptable salt thereof.

In certain other embodiments, the combination therapy comprises a deuterium-enriched compound described herein and a second therapeutic agent for treating cancer. Exemplary second therapeutic agents for treating cancer include, for example, an alkylating agent, an anti-metabolite (i.e., a molecule that impedes DNA and/or RNA synthesis), an anti-microtubule agent, a topoisomerase inhibitor, a cytotoxic antibiotic, a tyrosine kinase inhibitor, an inhibitor of tumor necrosis factor alpha, anti-neoplastic radiation therapy, or a Programmed Death protein-1 (PD-1) modulator (e.g., an inhibitor). In certain embodiments, the second therapeutic agent for treating cancer is azacitidine, azathioprine, bleomycin, carboplatin, capecitabine, carmustine, cisplatin, chlorambucil, cyclophosphamide, cytarabine, dacarbazine, daunorubicin, docetaxel, doxifluridine, doxorubicin, epirubicin, epothilone, etoposide, fluorouracil, fulvestrant, gemcitabine, hydroxyurea, idarubicin, imatinib, lomustine, mechlorethamine, mercaptopurine, methotrexate, mitoxantrone, oxaliplatin, paclitaxel, pemetrexed, procarbazine, raloxifene, teniposide, temozolomide, tamoxifen, toremifene, valrubicin, vinblastine, vincristine, vindesine, vinorelbine, or a pharmaceutically acceptable salt thereof.

In yet other embodiments, the second therapeutic agent for treating cancer is abraxane; acivicin; aclarubicin; acodazole hydrochloride; acronine; adozelesin; aldesleukin; altretamine; ambomycin; ametantrone acetate; amrubicin; amsacrine; anastrozole; anthramycin; asparaginase; asperlin; azacitidine; azetepa; azotomycin; batimastat; benzodepa; bicalutamide; bisantrene hydrochloride; bisnafide dimesylate: bizelesin; bleomycin sulfate; brequinar sodium; bropirimine; busulfan; cactinomycin; calusterone; caracemide; carbetimer; carboplatin; carmustine; carubicin hydrochloride; carzelesin; cedefmgol: celecoxib; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; de/.aguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatm; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; herceptin; hydroxyurea; idarubicin hydrochloride; ifosfamide; ilmofosine; iproplatin; irinotecan; irinotecan hydrochloride; lanreotide acetate; lapatinib; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; portiromycin; prednimustine; procarbazine hydrochloride; puromycin; puromycin hydrochloride; pyrazofurin; riboprine; romidepsin; safingol; safingol hydrochloride; semustine; simtrazene; sparfosate sodium; sparsomycin; spirogermanium hydrochloride; spiromustine; spiroplatin; a stem cell treatment; streptonigrin; streptozocin; sulofenur; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; or zorubicin hydrochloride.

Exemplary second active agents (or additional therapeutic agents) for treating bacterial infections include, for example, an aminoglycoside, carbacephem, carbapenem, cephalosporin (e.g., first generation, second generation, third generation, or fourth generation), a glycopeptide, lipopeptide, macrolide, monobactam, penicillin, polypeptide, quinolone, sulfonamide, tetracycline, oxazolidinone, rifamycin, and various unclassified antibiotics (e.g., chloramphenicol), each of which is described in more detail below.

Penicillins include those antibiotic drugs obtained from penicillium molds or produced synthetically, which are most active against Gram-positive bacteria and used in the treatment of various infections and diseases. Penicillin is one of the beta-lactam antibiotics, all of which possess a four-ring beta-lactam structure fused with a five-membered thiazolidine ring. These antibiotics are nontoxic and kill sensitive bacteria during their growth stage by the inhibition of biosynthesis of their cell wall mucopeptide. Penicillin antibiotics provide narrow spectrum bioactivity, moderate or intermediate spectrum bioactivity, and broad spectrum bioactivity. Without limitation, narrow spectrum penicillins include methicillin, dicloxacillin, flucloxacillin, oxacillin, nafcillin, or the like. Without limitation, moderate or intermediate spectrum penicillins include amoxicillin, ampicillin, or the like. Penicillins include, without limitation, ampicillin, azlocillin, carbenicillin, cloxacillin, dicloxacillin, flucloxacillin, mezlocillin, nafcillin, penicillin, piperacillin, and ticarcillin.

Aminoglycosides are a group of antibiotics that are effective against certain types of bacteria. They include amikacin, gentamicin, kanamycin, neomycin, netilmicin, paromomycin, streptomycin, tobramycin and apramycin. Aminoglycosides are useful primarily in infections involving aerobic, gram-negative bacteria, such as Pseudomonas, Acinetobacter, and Enterobacter. In addition, some mycobacteria, including the bacteria that cause tuberculosis, are susceptible to aminoglycosides.

Carbacephem is a class of antibiotic medications, specifically modified forms of cephalosporin. Without limitation, carbacephems include loracarbef, or the like.

Carbapenems are a class of beta-lactam antibiotics, which include, without limitation, imipenem (often given as part of imipenem/cilastatin), meropenem, ertapenem, faropenem, doripenem, panipenem/betamipron, and the like.

Cephalosporins are a class of beta-lactam antibiotics. Together with cephamycins they belong to a sub-group called cephems. First generation cephalosporins include, without limitation, cefadroxil, cefazolin, and cefalexin. Second generation cephalosporin typically have a greater gram-negative spectrum while retaining some activity against gram-positive cocci. Second generation cephalosporins include, for example, cefonicid, cefprozil, cefuroxime, cefuzonam, cefaclor, cefamandole, ceforanide, and cefotiam. Third generation cephalosporins typically have a broad spectrum of activity and further increased activity against gram-negative organisms. Without limitation, third generation cephalosporins include cefcapene, cefdaloxime, cefdinir, cefditoren, cefetamet, cefixime, cefmenoxime, cefodizime, cefoperazone, cefotaxime, cefpimizole, cefpodoxime, cefteram, ceftibuten, ceftiofur, ceftiolene, ceftizoxime, and ceftriaxone. Third generation cephalosporins with antipseudomonal activity include ceftazidime, cefpiramide, and cefsulodin. Oxacephems are also sometimes grouped with third-generation cephalosporins and include latamoxef and flomoxef. Fourth generation cephalosporins are extended-spectrum agents typically with similar activity against gram-positive organisms as first-generation cephalosporins. Exemplary fourth generation cephalosporins include cefclidine, cefepime, cefluprenam, cefoselis, cefozopran, cefpirome, and cefquinome. These cephems have progressed far enough to be named, but have not been assigned to a particular generation: ceftobiprole, cefaclomezine, cefaloram, cefaparole, cefcanel, cefedrolor, cefempidone, cefetrizole, cefivitril, cefmatilen, cefmepidium, cefovecin, cefoxazole, cefrotil, cefsumide, ceftioxide, ceftobiprole, ceftobiprole, and cefuracetime.

Glycopeptide antibiotics feature a glycosylated cyclic or polycyclic nonribosomal peptide. Exemplary glycopeptide antibiotics include vancomycin, teicoplanin, ramoplanin, and decaplanin.

Macrolides are a group of drugs (typically antibiotics) whose activity stems from the presence of a macrolide ring, a large lactone ring to which one or more deoxy sugars, usually cladinose and desosamine, are attached. The lactone ring can be either 14-, 15- or 16-membered. Common antibiotic macrolides include erythromycin, azithromycin, troleandomycin, clarithromycin, dirithromycin, and roxithromycin.

Monobactams are beta-lactam antibiotics wherein the beta-lactam ring is alone, and not fused to another ring (in contrast to most other beta-lactams, which have at least two rings). An example is aztreonam.

Polypeptide antibiotics include bacitracin, colistin, and polymyxin B.

Quinolones are another family of broad spectrum antibiotics. The parent of the group is nalidixic acid. Exemplary quinolone antibiotics include cinoxacin, flumequine, nalidixic acid, oxolinic acid, piromidic acid, pipemidic acid, ciprofloxacin, enoxacin, fleroxacin, lomefloxacin, nadifloxacin, norfloxacin, ofloxacin, pefloxacin, rufloxacin, balofloxacin, grepafloxacin, levofloxacin, pazufloxacin mesilate, sparfloxacin, temafloxacin, tosufloxacin, clinafloxacin, gemifloxacin, moxifloxacin, gatifloxacin, sitafloxacin, and trovafloxacin.

Antibacterial sulfonamides (sometimes called simply sulfa drugs) are synthetic antimicrobial agents that contain the sulfonamide group. In bacteria, antibacterial sulfonamides act as competitive inhibitors of the enzyme dihydropteroate synthetase, DHPS. Several antibacterial sulfonamides include, for example, mafenide prontosil, sulfacetamide, sulfamethizole, sulfanilamide, sulfasalazine, sulfisoxazole, sulfamethoxazole, and trimethoprim-sulfamethoxazole.

Tetracyclines are a group of broad-spectrum antibiotics named for their four (“tetra-”) hydrocarbon rings (“-cycl-”) derivation (“-ine”). Exemplary tetracyclines include tetracycline, chlortetracycline, oxytetracycline, demeclocycline, doxycycline, lymecycline, meclocycline, methacycline, minocycline, rolitetracycline, and tigecycline.

Oxazolidinones are a class of compounds containing 2-oxazolidone in their structures. Oxazolidinones are useful antibiotics. Some of the most important oxazolidinones are the last generation of antibiotics used against gram-positive bacterial strains. One example of an oxazolidinone is linezolid.

Rifamycins are a group antibiotics that are synthesized either naturally by the bacteria Amycolatopsis mediterranei or Amycolatopsis rifamycinica, or artificially. Rifamycins are particularly effective against mycobacteria, and are therefore used to treat tuberculosis, leprosy, and Mycobacterium avium complex (MAC) infections. The rifamycin antibiotic group includes, without limitation, rifampin.

Lipopeptide antibiotics includes peptides with attached lipids or a mixture of lipids and peptides such as the cyclic lipopeptide, daptomycin.

Other unclassified antibiotics include chloramphenicol, clindamycin, ethambutol, fosfomycin, furazolidone, isoniazid, metronidazole, mupirocin, nitrofurantoin, platensimycin, pyrazinamide, quinupristin/dalfopristin, spectinomycin, and telithromycin.

Exemplary second active agents (or additional therapeutic agents) for treating fungal infections include, for example, 2-phenylphenol; 8-hydroxyquinoline sulfate; acibenzolar-S-methyl; aldimorph; amidoflumet; ampropylfos; ampropylfos-potassium; andoprim; anilazine; azaconazole; azoxystrobin; benalaxyl; benodanil; benomyl; benthiavalicarb-isopropyl; benzamacril; benzamacril-isobutyl; bilanafos; binapacryl; biphenyl; bitertanol; blasticidin-S; bromuconazole; bupirimate; buthiobate; butylamine; calcium polysulphide; capsimycin; captafol; captan; carbendazim; carboxin; carpropamid; carvone; chinomethionat; chlobenthiazone; chlorfenazole; chloroneb; chlorothalonil; chlozolinate; clozylacon; cyazofamid; cyflufenamid; cymoxanil; cyproconazole; cyprodinil; cyprofuram; Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-M; dinocap; diphenylamine; dipyrithione; ditalimfos; dithianon; dodine; drazoxolon; edifenphos; epoxiconazole; ethaboxam; ethirimol; etridiazole; famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulphamide, flutolanil; flutriafol; folpet; fosetyl-A1; fosetyl-sodium; fuberidazole; furalaxyl; furametpyr; furcarbanil; furmecyclox; guazatine; hexachlorobenzene; hexaconazole; hymexazole; imazalil; imibenconazole; iminoctadine triacetate; iminoctadine tris(albesil); iodocarb; ipconazole; iprobenfos; iprodione; iprovalicarb; irumamycin; isoprothiolane; isovaledione; kasugamycin; kresoxim-methyl; mancozeb; maneb; meferimzone; mepanipyrim; mepronil; metalaxyl; metalaxyl-M; metconazole; methasulphocarb; methfuroxam; metiram; metominostrobin; metsulphovax; mildiomycin; myclobutanil; myclozolin; natamycin; nicobifen; nitrothal-isopropyl; noviflumuron; nuarimol; ofurace; orysastrobin; oxadixyl; oxolinic acid; oxpoconazole; oxycarboxin; oxyfenthiin; paclobutrazole; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolenitrine; quinconazole; quinoxyfen; quintozene; simeconazole; spiroxamine; sulphur; tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole; uniconazole; validamycin A; vinclozolin; zineb; ziram; zoxamide; (2S)—N-[2-[4-[[3-(4-chlorophenyl)-2-propynyl]oxy]-3-methoxyphenyl]ethyl]-3-methyl-2-[(methylsulphonyl)amino]-butanamide; 1-(1-naphthalenyl-1H-pyrrole-2,5-dione; 2,3,5,6-tetrachloro-4-(methylsulphonyl)-pyridine; 2-amino-4-methyl-N-phenyl-5-thiazolecarboxamide; 2-chloro-N-(2,3-dihydro-1,1,3-trimethyl-1H-inden-4-yl)-3-pyridinecarboxamide; 3,4,5-trichloro-2,6-pyridinedicarbonitrile; actinovate; cis-1-(4-chlorophenyl)-2-(1H-1,2,4-triazol-1-yl)cycloheptanol; methyl 1-(2,3-dihydro-2,2-dimethyl-1H-inden-1-yl)-1H-imidazole-5-carboxylate; monopotassium carbonate; N-6-methoxy-3-pyridinyl)-cyclopropanecarboxamide; N-butyl-8-(1,1-dimethylethyl)-1-oxaspiro-[4.5]decane-3-amine; sodium tetrathiocarbonate; and copper salts and preparations, such as Bordeaux mixture; copper hydroxide; copper naphthenate; copper oxychloride; copper sulfate; cufraneb; copper oxide; mancopper; oxine-copper. Bactericides: bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate, and other copper preparations.

Administration of a compound provided herein, or a pharmaceutically acceptable salt thereof, and the second active agent(s) to a patient can occur simultaneously or sequentially by the same or different routes of administration. The suitability of a particular route of administration employed for a particular active agent will depend on the active agent itself (e.g., whether it can be administered orally without decomposing prior to entering the blood stream) and the disease being treated. One route of administration for compounds provided herein is oral. Routes of administration for the second active agents or ingredients are known to those of ordinary skill in the art. See, e.g., Physicians' Desk Reference (60^{th} Ed., 2006).

### IV. Pharmaceutical Compositions

The invention provides pharmaceutical compositions comprising a deuterium-enriched compound described herein, such as a compound of Formula I or II, and a pharmaceutically acceptable carrier. In certain embodiments, the pharmaceutical compositions comprise a therapeutically-effective amount of a deuterium-enriched compound described herein, such as a compound of Formula I or II, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Pharmaceutical compositions can be used in the preparation of individual, single unit dosage forms. Pharmaceutical compositions and dosage forms provided herein comprise a compound provided herein, or a pharmaceutically acceptable salt thereof Pharmaceutical compositions and dosage forms can further comprise one or more excipients. Additionally, pharmaceutical compositions and dosage forms provided herein can comprise one or more additional active ingredients. Examples of optional second, or additional, active ingredients are described above.

Single unit dosage forms provided herein are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal or transcutaneous administration to a patient. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or water-in-oil liquid emulsions), solutions, and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape, and type of dosage forms will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms are used will vary from one another and will be readily apparent to those skilled in the art. See, e.g., Remington's Pharmaceutical Sciences, 18th ed., Mack Publishing, Easton Pa. (1990).

The suitability of a particular excipient may depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided are pharmaceutical compositions and dosage forms that contain little, if any, lactose or other mono- or disaccharides. As used herein, the term “lactose-free” means that the amount of lactose present, if any, is insufficient to substantially increase the degradation rate of an active ingredient. Lactose-free compositions can comprise excipients that are well known in the art and are listed, for example, in the U.S. Pharmacopeia (USP) 25-NF20 (2002). In general, lactose-free compositions comprise active ingredients, a binder/filler, and a lubricant in pharmaceutically compatible and pharmaceutically acceptable amounts. In another aspect, lactose-free dosage forms comprise active ingredients, microcrystalline cellulose, pre-gelatinized starch, and magnesium stearate.

Also provided are anhydrous pharmaceutical compositions and dosage forms comprising active ingredients. Anhydrous pharmaceutical compositions and dosage forms can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected. An anhydrous pharmaceutical composition should be prepared and stored such that its anhydrous nature is maintained. Accordingly, anhydrous compositions are, in another aspect, packaged using materials known to prevent exposure to water such that they can be included in suitable formulary kits. Examples of suitable packaging include, but are not limited to, hermetically sealed foils, plastics, dose containers (e.g., vials), blister packs, and strip packs.

Also provided are pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. In another aspect, dosage forms comprise a compound provided herein in an amount of from about 0.10 to about 500 mg. Examples of dosages include, but are not limited to, 0.1, 1, 2, 5, 7.5, 10, 12.5, 15, 17.5, 20, 25, 50, 100, 150, 200, 250, 300, 350, 400, 450, or 500 mg.

In another aspect, dosage forms comprise the second active ingredient in an amount of 1 to about 1000 mg, from about 5 to about 500 mg, from about 10 to about 350 mg, or from about 50 to about 200 mg. Of course, the specific amount of the second active agent will depend on the specific agent used, the diseases or disorders being treated or managed, and the amount(s) of a compound provided herein, and any optional additional active agents concurrently administered to the patient.

Pharmaceutical compositions that are suitable for oral administration can be provided as discrete dosage forms, such as, but not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Oral dosage forms provided herein are prepared by combining the active ingredients in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In another aspect, the invention provides oral dosage forms that are tablets or capsules, in which case solid excipients are employed. In another aspect, the tablets can be coated by standard aqueous or non-aqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinylpyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions is, in another aspect, present in from about 50 to about 99 weight percent of the pharmaceutical composition or dosage form.

Disintegrants may be used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients may be used to form solid oral dosage forms. The amount of disintegrant used varies based upon the type of formulation, and is readily discernible to those of ordinary skill in the art. In another aspect, pharmaceutical compositions comprise from about 0.5 to about 15 weight percent of disintegrant, or from about 1 to about 5 weight percent of disintegrant. Disintegrants that can be used in pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, and mixtures thereof. Additional lubricants include, for example, a Syloid® silica gel (AEROSIL200, manufactured by W. R. Grace Co. of Baltimore, Md.), a coagulated aerosol of synthetic silica (marketed by Degussa Co. of Piano, Tex.), CAB-O-SIL® (a pyrogenic silicon dioxide product sold by Cabot Co. of Boston, Mass.), and mixtures thereof. If used at all, lubricants may be used in an amount of less than about 1 weight percent of the pharmaceutical compositions or dosage forms into which they are incorporated.

In another aspect, the invention provides a solid oral dosage form comprising a compound provided herein, anhydrous lactose, microcrystalline cellulose, polyvinylpyrrolidone, stearic acid, colloidal anhydrous silica, and gelatin.

Active ingredients provided herein can also be administered by controlled release means or by delivery devices that are well known to those of ordinary skill in the art. Examples include, but are not limited to, those described in U.S. Pat. Nos. 3,845,770; 3,916,899; 3,536,809; 3,598,123; 4,008,719, 5,674,533, 5,059,595, 5,591,767, 5,120,548, 5,073,543, 5,639,476, 5,354,556, and 5,733,566, each of which is incorporated in its entirety herein by reference. Such dosage forms can be used to provide slow or controlled-release of one or more active ingredients using, for example, hydroxypropyl methyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active agents provided herein. In another aspect, the invention provides single unit dosage forms suitable for oral administration such as, but not limited to, tablets, capsules, gel caps, and caplets that are adapted for controlled-release.

Parenteral dosage forms can be administered to patients by various routes including, but not limited to, subcutaneous, intravenous (including bolus injection), intramuscular, and intraarterial. Administration of a parenteral dosage form bypasses a patient's natural defenses against contaminants, and thus, in these aspects, parenteral dosage forms are sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms. For example, cyclodextrin and its derivatives can be used to increase the solubility of a compound provided herein. See, e.g., U.S. Pat. No. 5,134,127, which is incorporated in its entirety herein by reference.

Topical and mucosal dosage forms provided herein include, but are not limited to, sprays, aerosols, solutions, emulsions, suspensions, eye drops or other ophthalmic preparations, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels.

Suitable excipients (e.g., carriers and diluents) and other materials that can be used to provide topical and mucosal dosage forms encompassed herein are well known to those skilled in the pharmaceutical arts, and depend on the particular tissue to which a given pharmaceutical composition or dosage form will be applied. In another aspect, excipients include, but are not limited to, water, acetone, ethanol, ethylene glycol, propylene glycol, butane-1,3-diol, isopropyl myristate, isopropyl palmitate, mineral oil, and mixtures thereof to form solutions, emulsions or gels, which are nontoxic and pharmaceutically acceptable. Moisturizers or humectants can also be added to pharmaceutical compositions and dosage forms. Examples of additional ingredients are well known in the art. See, e.g., Remington's Pharmaceutical Sciences, 16th and 18th eds., Mack Publishing, Easton Pa. (1980 & 1990).

The pH of a pharmaceutical composition or dosage form may also be adjusted to improve delivery of one or more active ingredients. Also, the polarity of a solvent carrier, its ionic strength, or tonicity can be adjusted to improve delivery. Compounds such as stearates can also be added to pharmaceutical compositions or dosage forms to alter the hydrophilicity or lipophilicity of one or more active ingredients so as to improve delivery. In other aspects, stearates can serve as a lipid vehicle for the formulation, as an emulsifying agent or surfactant, or as a delivery-enhancing or penetration-enhancing agent. In other aspects, salts of the active ingredients can be used to further adjust the properties of the resulting composition.

In another aspect, the active ingredients provided herein are not administered to a patient at the same time or by the same route of administration. In another aspect, provided are kits which can simplify the administration of appropriate amounts of active ingredients.

In another aspect, the invention provides a kit comprising a dosage form of a compound provided herein. Kits can further comprise additional active ingredients or a pharmacologically active mutant or derivative thereof, or a combination thereof. Examples of the additional active ingredients include, but are not limited to, those disclosed herein.

In other aspects, the kits can further comprise devices that are used to administer the active ingredients. Examples of such devices include, but are not limited to, syringes, drip bags, patches, and inhalers.

### V. Definitions

To facilitate an understanding of the present invention, a number of terms and phrases are defined below.

The term “compound” refers to a quantity of molecules that is sufficient to be weighed, tested for its structural identity, and to have a demonstrable use (e.g., a quantity that can be shown to be active in an assay, an in vitro test, or in vivo test, or a quantity that can be administered to a patient and provide a therapeutic benefit).

Unless indicated otherwise, when a D is specifically recited at a position or is shown in a formula, this D represents a mixture of hydrogen and deuterium where the amount of deuterium is about 100% (i.e., the abundance of deuterium ranges from greater than 90% up to 100%). In certain embodiments, the abundance of deuterium in D is from 95% to 100%, or from 97% to 100%.

The term “patient” refers to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

“Therapeutically effective amount” includes an amount of a compound of the invention that is effective when administered alone or in combination to treat the desired condition or disorder. “Therapeutically effective amount” includes an amount of the combination of compounds claimed that is effective to treat the desired condition or disorder. The combination of compounds can be additive and is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower incidence of adverse side effects and/or toxicity, increased efficacy, or some other beneficial effect of the combination compared with the individual components.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of the basic residues. The pharmaceutically acceptable salts include the conventional quaternary ammonium salts of the parent compound formed, for example, from non-toxic inorganic or organic acids. These salts can be prepared in situ in the administration vehicle or the dosage form manufacturing process, or by separately reacting a purified compound of the invention in its free base form with a suitable organic or inorganic acid, and isolating the salt thus formed during subsequent purification. For example, such conventional non-toxic salts include, but are not limited to, those derived from inorganic and organic acids selected from 1,2-ethanedisulfonic, 2-acetoxybenzoic, 2-hydroxyethanesulfonic, acetic, ascorbic, benzenesulfonic, benzoic, bicarbonic, bisulfuric, carbonic, citric, edetic, ethanedisulfonic, ethanesulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauric, lauryl sulfonic, maleic, malic, mandelic, methanesulfonic, napsylic, naphthylic, nitric, oleic, oxalic, palmitic, pamoic, pantothenic, phenylacetic, phosphoric, polygalacturonic, propionic, salicylic, stearic, succinic, sulfamic, sulfanilic, sulfuric, tannic, tartaric, toluenesulfonic, and valeric. (See, for example, Berge et al. (1977) “Pharmaceutical Salts”, J. Pharm. Sci. 66:1-19.). In certain embodiments, the pharmaceutically acceptable salt is a hydrochloric acid salt.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or a water/oil emulsion), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, if a variable is not accompanied by a definition, then the previous definition of the variable controls.

Finally, the invention may be embodied in other specific forms without departing from the spirit or essential attributes thereof. This invention encompasses all combinations of aspects and embodiments of the invention noted herein. It is understood that any and all aspects of the invention may be taken in conjunction with any other aspects and/or embodiments to describe additional aspects. It is also to be understood that each individual element of the aspects is intended to be taken individually as its own independent aspect. Furthermore, any element of an aspect is meant to be combined with any and all other elements from any aspect to describe an additional aspect.

### INCORPORATION BY REFERENCE

All references listed herein are individually incorporated in their entirety by reference.

### EQUIVALENTS

Numerous modifications and variations of the invention are possible in light of the above teachings. It is therefore to be understood that within the scope of the appended claims, the invention may be practiced otherwise that as specifically described herein.

1-72. (canceled)

73. A compound of Formula I-E or II-E: 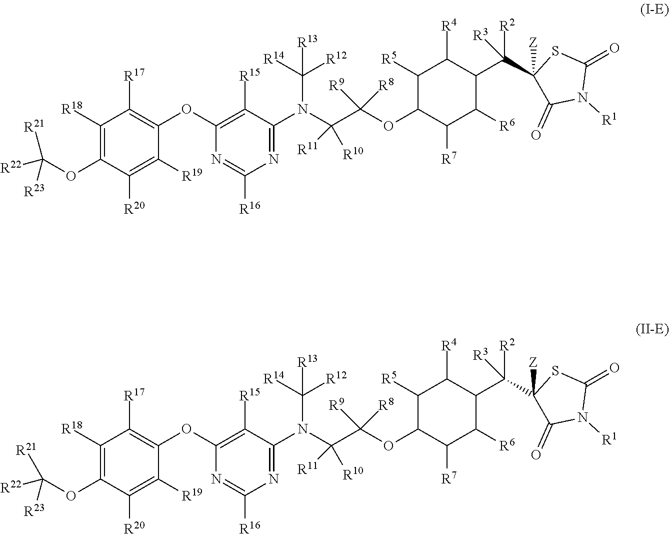
or a pharmaceutically acceptable salt thereof, wherein:
Z is H or D, provided that the abundance of deuterium in Z is at least 30%;
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²² and R²³ are independently H or D; and
the compound has an enantiomeric excess of at least 75%.

74. The compound of claim 73, wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², and R²³ are H.

75. The compound of claim 73, wherein the compound is 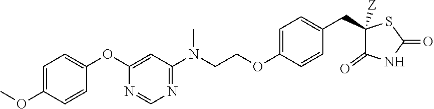
having a stereochemical purity of at least 85% enantiomeric excess, or a pharmaceutically acceptable salt thereof.

76. The compound of claim 75, wherein the abundance of deuterium in Z is at least 60%.

77. The compound of claim 75, wherein the abundance of deuterium in Z is at least 90%.

78. The compound of claim 75, wherein the compound has a stereochemical purity of at least 90% enantiomeric excess.

79. The compound of claim 73, wherein the compound is 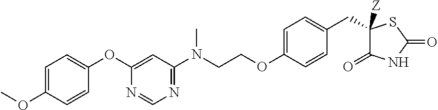
having a stereochemical purity of at least 85% enantiomeric excess, or a pharmaceutically acceptable salt thereof.

80. The compound of claim 79, wherein the abundance of deuterium in Z is at least 60%.

81. The compound of claim 79, wherein the abundance of deuterium in Z is at least 90%.

82. The compound of claim 79, wherein the compound has a stereochemical purity of at least 90% enantiomeric excess.

83. A pharmaceutical composition comprising a compound of claim 73 and a pharmaceutically acceptable carrier.

84. A compound of Formula I-J or Formula II-J: 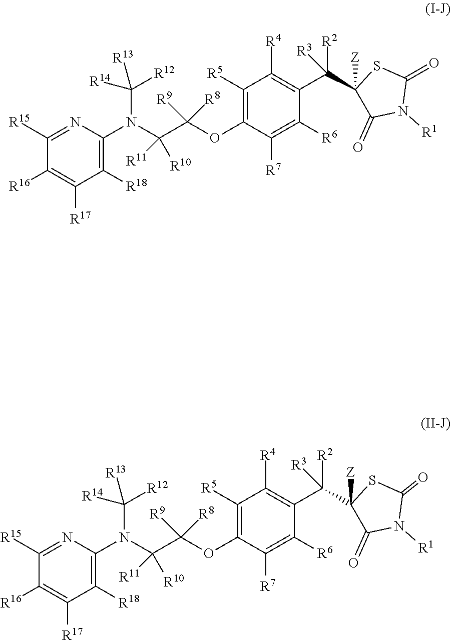
or a pharmaceutically acceptable salt thereof, wherein:
Z is H or D, provided that the abundance of deuterium in Z is at least 30%;
R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are independently H or D; and
the compound has a stereochemical purity of at least 75% enantiomeric excess at the carbon atom bearing variable Z.

85. The compound of claim 84, wherein R¹, R², R³, R⁴, R⁵, R⁶, R⁷, R⁸, R⁹, R¹⁰, R¹¹, R¹², R¹³, R¹⁴, R¹⁵, R¹⁶, R¹⁷, and R¹⁸ are H.

86. The compound of claim 84, wherein the compound is 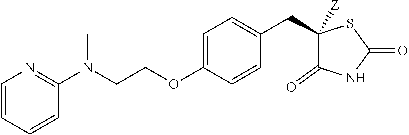
having a stereochemical purity of at least 85% enantiomeric excess, or a pharmaceutically acceptable salt thereof.

87. The compound of claim 86, wherein the abundance of deuterium in Z is at least 60%.

88. The compound of claim 86, wherein the abundance of deuterium in Z is at least 90%.

89. The compound of claim 86, wherein the compound has a stereochemical purity of at least 90% enantiomeric excess.

90. The compound of claim 84, wherein the compound is 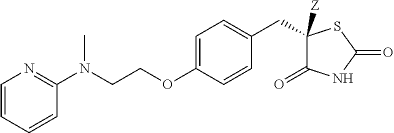
having a stereochemical purity of at least 85% enantiomeric excess, or a pharmaceutically acceptable salt thereof.

91. The compound of claim 90, wherein the abundance of deuterium in Z is at least 60%.

92. The compound of claim 90, wherein the abundance of deuterium in Z is at least 90%.

93. The compound of claim 90, wherein the compound has a stereochemical purity of at least 90% enantiomeric excess.

94. A pharmaceutical composition comprising a compound of claim 84 and a pharmaceutically acceptable carrier.

95. A method of treating a medical disorder in a patient, comprising administering to a patient in need thereof a therapeutically effective amount of a compound of claim 73 to treat the disorder.
